SEQUENCE LISTING (23 sequences)

SEQ ID NO: 1 - DNA, 1221 bp, Escherichia coli
atgaaacgtgcagtgattactggcctgggcattgtttccagcatcggtaataaccagcaggaagtcctggcatctctgcgtgaaggacgttcagggatcactttctctcaggagctgaaggattccggcatgcgtagccacgtctggggcaacgtaaaactggataccactggcctcattgaccgcaaagttgtgcgctttatgagcgacgcatccatttatgcattcctttctatggagcaggcaatcgctgatgcgggcctctctccggaagcttaccagaataacccgcgcgttggcctgattgcaggttccggcggcggctccccgcgtttccaggtgttcggcgctgacgcaatgcgcggcccgcgcggcctgaaagcggttggcccgtatgtggtcaccaaagcgatggcatccggcgtttctgcctgcctcgccaccccgtttaaaattcatggcgttaactactccatcagctccgcgtgtgcgacttccgcacactgtatcggtaacgcagtagagcagatccaactgggcaaacaggacatcgtgtttgctggcggcggcgaagagctgtgctgggaaatggcttgcgaattcgacgcaatgggtgcgctgtctactaaatacaacgacaccccggaaaaagcctcccgtacttacgacgctcaccgtgacggtttcgttatcgctggcggcggcggtatggtagtggttgaagagctggaacacgcgctggcgcgtggtgctcacatctatgctgaaatcgttggctacggcgcaacctctgatggtgcagacatggttgctccgtctggcgaaggcgcagtacgctgcatgaagatggcgatgcatggcgttgataccccaatcgattacctgaactcccacggtacttcgactccggttggcgacgtgaaagagctggcagctatccgtgaagtgttcggcgataagagcccggcgatttctgcaaccaaagccatgaccggtcactctctgggcgctgctggcgtacaggaagctatctactctctgctgatgctggaacacggctttatcgccccgagcatcaacattgaagagctggacgagcaggctgcgggtctgaacatcgtgaccgaaacgaccgatcgcgaactgaccaccgttatgtctaacagcttcggcttcggcggcaccaacgccacgctggtaatgcgcaagctgaaagattaa

SEQ ID NO: 2 - protein, 406 aa, Escherichia coli
MKRAVITGLGIVSSIGNNQQEVLASLREGRSGITFSQELKDSGMRSHVWGNVKLDTTGLIDRKVVRFMSDASIYAFLSMEQAIADAGLSPEAYQNNPRVGLIAGSGGGSPRFQVFGADAMRGPRGLKAVGPYVVTKAMASGVSACLATPFKIHGVNYSISSACATSAHCIGNAVEQIQLGKQDIVFAGGGEELCWEMACEFDAMGALSTKYNDTPEKASRTYDAHRDGFVIAGGGGMVVVEELEHALARGAHIYAEIVGYGATSDGADMVAPSGEGAVRCMKMAMHGVDTPIDYLNSHGTSTPVGDVKELAAIREVFGDKSPAISATKAMTGHSLGAAGVQEAIYSLLMLEHGFIAPSINIEELDEQAAGLNIVTETTDRELTTVMSNSFGFGGTNATLVMRKLKD

SEQ ID NO: 3 - DNA, 1245 bp, Escherichia coli
atggtgtctaagcgtcgtgtagttgtgaccggactgggcatgttgtctcctgtcggcaataccgtagagtctacctggaaagctctgcttgccggtcagagtggcatcagcctaatcgaccatttcgatactagcgcctatgcaacgaaatttgctggcttagtaaaggattttaactgtgaggacattatctcgcgcaaagaacagcgcaagatggatgccttcattcaatatggaattgtcgctggcgttcaggccatgcaggattctggccttgaaataacggaagagaacgcaacccgcattggtgccgcaattggctccgggattggcggcctcggactgatcgaagaaaaccacacatctctgatgaacggtggtccacgtaagatcagcccattcttcgttccgtcaacgattgtgaacatggtggcaggtcatctgactatcatgtatggcctgcgtggcccgagcatctctatcgcgactgcctgtacttccggcgtgcacaacattggccatgctgcgcgtattatcgcgtatggcgatgctgacgtgatggttgcaggtggcgcagagaaagccagtacgccgctgggcgttggtggttttggcgcggcacgtgcattatctacccgcaatgataacccgcaagcggcgagccgcccgtgggataaagagcgtgatggtttcgtactgggcgatggtgccggtatgctggtacttgaagagtacgaacacgcgaaaaaacgcggtgcgaaaatttacgctgaactcgtcggctttggtatgagcagcgatgcttatcatatgacgtcaccgccagaaaatggcgcaggcgcagctctggcgatggcaaatgctctgcgtgatgcaggcattgaagcgagtcagattggctacgttaacgcgcacggtacttctacgccggctggcgataaagctgaagcgcaggcggtgaaaaccatcttcggtgaagctgcaagccgtgtgttggtaagctccacgaaatctatgaccggtcacctgttaggtgcggcgggtgcagtagaatctatctactccatcctggcgctgcgcgatcaggctgttccgccaaccatcaacctggataacccggatgaaggttgcgatctggatttcgtaccgcacgaagcgcgtcaggttagcggaatggaatacactctgtgtaactccttcggcttcggtggcactaatggttctttgatctttaaaaagatctaa

SEQ ID NO: 4 - protein, 413 aa, Escherichia coli
MSKRRVVVTGLGMLSPVGNTVESTWKALLAGQSGISLIDHFDTSAYATKFAGLVKDFNCEDIISRKEQRKMDAFIQYGIVAGVQAMQDSGLEITEENATRIGAAIGSGIGGLGLIEENHTSLMNGGPRKISPFFVPSTIVNMVAGHLTIMYGLRGPSISIATACTSGVHNIGHAARIIAYGDADVMVAGGAEKASTPLGVGGFGAARALSTRNDNPQAASRPWDKERDGFVLGDGAGMLVLEEYEHAKKRGAKIYAELVGFGMSSDAYHMTSPPENGAGAALAMANALRDAGIEASQIGYVNAHGTSTPAGDKAEAQAVKTIFGEAASRVLVSSTKSMTGHLLGAAGAVESIYSILALRDQAVPPTINLDNPDEGCDLDFVPHEARQVSGMEYTLCNSFGFGGTNGSLIFKKI

SEQ ID NO: 5 - DNA, 627 bp, Escherichia coli
atgatgaacttcaacaatgttttccgctggcatttgcccttcctgttcctggtcctgttaaccttccgtgccgccgcagcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgcctgcaaactatggtcgccgttataatgaagcctttagcgccatttaccccaaactcgccaaagagtttgatgttccgctgctgcccttttttatggaagaggtctacctcaagccacaatggatgcaggatgacggtattcatcccaaccgcgacgcccagccgtttattgccgactggatggcgaagcagttgcagcctttagtaaatcatgactcataa

SEQ ID NO: 6 - protein, 208 aa, Escherichia coli
MMNFNNVFRWHLPFLFLVLLTFRAAAADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 7 - DNA, 552 bp, Escherichia coli
atggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgcctgcaaactatggtcgccgttataatgaagcctttagcgccatttaccccaaactcgccaaagagtttgatgttccgctgctgcccttttttatggaagaggtctacctcaagccacaatggatgcaggatgacggtattcatcccaaccgcgacgcccagccgtttattgccgactggatggcgaagcagttgcagcctttagtaaatcatgactcataa

SEQ ID NO: 8 - protein, 183 aa, Escherichia coli
MADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 9 - DNA, 3522 bp, Mycobacterium smegmatis
atgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctga

SEQ ID NO: 10 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 11 - DNA, 519 bp, Escherichia coli
atggtagataaacgcgaatcctatacaaaagaagaccttcttgcctctggtcgcggtgaactgtttggcgctaaaggcccgcaattgccagcaccgaacatgctgatgatggaccgtgtggtcaaaatgaccgaaacgggtggtaacttcgacaaagggtatgttgaagcagaactggatatcaatccggatctgtggttcttcggatgccactttattggcgatccggttatgccgggatgcctgggcctggacgcaatgtggcagctggtagggttctacctcggctggctgggcggcgaaggtaaaggccgcgcgctgggcgttggcgaagtgaaattcactggtcaggtactgccgacagcgaaaaaagtgacctaccgtattcactttaaacgcattgttaaccgtcgtctgattatgggcctggcggatggcgaagtgctggttgatggtcgtctgatctataccgccagcgacctgaaagtcggtctgttccaggatacgtctgccttctga

SEQ ID NO: 12 - protein, 172 aa, Escherichia coli
MVDKRESYTKEDLLASGRGELFGAKGPQLPAPNMLMMDRVVKMTETGGNFDKGYVEAELDINPDLWFFGCHFIGDPVMPGCLGLDAMWQLVGFYLGWLGGEGKGRALGVGEVKFTGQVLPTAKKVTYRIHFKRIVNRRLIMGLADGEVLVDGRLIYTASDLKVGLFQDTSAF

SEQ ID NO: 13 - DNA, 459 bp, Escherichia coli
atgttgactactaacactcatactctgcagattgaagagattttagaacttctgccgcaccgtttcccgttcttactggtggatcgcgtgctggattttgaagaaggtcgttttctgcgcgcagtaaaaaatgtctctgtcaatgagccattcttccagggccatttccctggaaaaccgattttcccgggtgtgctgattctggaagcaatggcacaggcaacaggtattctggcgtttaaaagcgtaggaaaactggaaccgggtgagctgtactacttcgctggtattgacgaagcgcgcttcaagcgcccggtcgtgcctggcgatcaaatgatcatggaagtcactttcgaaaaaacgcgccgcggcctgacccgttttaaaggggttgctctggtcgatggtaaagtagtttgcgaagcaacgatgatgtgtgctcgtagccgggaggcctga

SEQ ID NO: 14 - protein, 151 aa, Escherichia coli
MTTNTHTLQIEEILELLPHRFPFLLVDRVLDFEEGRFLRAVKNVSVNEPFFQGHFPGKPIFPGVLILEAMAQATGILAFKSVGKLEPGELYYFAGIDEARFKRPVVPGDQMIMEVTFEKTRRGLTRFKGVALVDGKVVCEATMMCARSREA

SEQ ID NO: 15 - DNA, 3522 bp, Artificial Sequence
  source: /note=“Description of Artificial Sequence Synthetic polynucleotide”
atgacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttag

SEQ ID NO: 16 - DNA, 552 bp, Artificial Sequence
  source: /note=“Description of Artificial Sequence Synthetic polynucleotide”
atggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggctgtgacggtttgcgtggttttcagccacagcaaaccgagcaaacgctgcgccagattttgcaggatgtcaaagccgccaacgctcttccattgttaatgcaaatacgtctgccttacaactatggtcgtcgttataatgaagcctttagcgccatttaccccaaactcgccaaagagtttgatgttccgctgctgcccttttttatggaagaggtctgcctcaagccacaatggatgcaggatgacggtattcatcccaaccgcgacgcccagccgtttattgccgactggatggcgaagcagttgcagcctttaaccaatcatgactcataa

SEQ ID NO: 17 - protein, 183 aa, Artificial Sequence
  source: /note=“Description of Artificial Sequence Synthetic polypeptide”
MADTLLILGDSLSAGYRMSASAAWPALLNDKWQSKTSVVNASISGDTSQQGLARLPALLKQHQPRWVLVELGGCDGLRGFQPQQTEQTLRQILQDVKAANALPLLMQIRLPYNYGRRYNEAFSAIYPKLAKEFDVPLLPFFMEEVCLKPQWMQDDGIHPNRDAQPFIADWMAKQLQPLTNHDS

SEQ ID NO: 18 - DNA, 552 bp, Artificial Sequence
  source: /note=“Description of Artificial Sequence Synthetic polynucleotide”
atggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgccttacaactatggtcgtcgttataatgaagcctttagcgccatttaccccaaactcgccaaagagtttgatgttccgctgctgcccttttttatggaagaggtctgcctcaagccacaatggatgcaggatgacggtattcatcccaaccgcgacgcccagccgtttattgccgactggatggcgaagcagttgcagcctttagtaaatcatgactcataa

SEQ ID NO: 19 - protein, 183 aa, Artificial Sequence
  source: /note=“Description of Artificial Sequence Synthetic polypeptide”
MADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPYNYGRRYNEAFSAIYPKLAKEFDVPLLPFFMEEVCLKPQWMQDDGIHPNRDAQPFIADWMAKQLQPLVNHDS

SEQ ID NO: 20 - DNA, 3507 bp, Mycobacterium tuberculosis
atgtcgatcaacgatcagcgactgacacgccgcgtcgaggacctatacgccagcgacgcccagttcgccgccgccagtcccaacgaggcgatcacccaggcgatcgaccagcccggggtcgcgcttccacagctcatccgtatggtcatggagggctacgccgatcggccggcactcggccagcgtgcgctccgcttcgtcaccgaccccgacagcggccgcaccatggtcgagctactgccgcggttcgagaccatcacctaccgcgaactgtgggcccgcgccggcacattggccaccgcgttgagcgctgagcccgcgatccggccgggcgaccgggtttgcgtgctgggcttcaacagcgtcgactacacaaccatcgacatcgcgctgatccggttgggcgccgtgtcggttccactgcagaccagtgcgccggtcaccgggttgcgcccgatcgtcaccgagaccgagccgacgatgatcgccaccagcatcgacaatcttggcgacgccgtcgaagtgctggccggtcacgccccggcccggctggtcgtattcgattaccacggcaaggttgacacccaccgcgaggccgtcgaagccgcccgagctcggttggccggctcggtgaccatcgacacacttgccgaactgatcgaacgcggcagggcgctgccggccacacccattgccgacagcgccgacgacgcgctggcgctgctgatttacacctcgggtagtaccggcgcacccaaaggcgccatgtatcgcgagagccaggtgatgagcttctggcgcaagtcgagtggctggttcgagccgagcggttacccctcgatcacgctgaacttcatgccgatgagccacgtcgggggccgtcaggtgctctacgggacgctttccaacggcggtaccgcctacttcgtcgccaagagcgacctgtcgacgctgttcgaggacctcgccctggtgcggcccacagaattgtgcttcgtgccgcgcatctgggacatggtgttcgcagagttccacagcgaggtcgaccgccgcttggtggacggcgccgatcgagcggcgctggaagcgcaggtgaaggccgagctgcgggagaacgtgctcggcggacggtttgtcatggcgctgaccggttccgcgccgatctccgctgagatgacggcgtgggtcgagtccctgctggccgacgtgcatttggtggagggttacggctccaccgaggccgggatggtcctgaacgacggcatggtgcggcgccccgcggtgatcgactacaagctggtcgacgtgcccgagctgggctacttcggcaccgatcagccctacccccggggcgagctgctggtcaagacgcaaaccatgttccccggctactaccagcgcccggatgtcaccgccgaggtgttcgaccccgacggcttctaccggaccggggacatcatggccaaagtaggccccgaccagttcgtctacctcgaccgccgcaacaacgtgctaaagctctcccagggcgagttcatcgccgtgtcgaagctcgaggcggtgttcggcgacagcccgctggtccgacagatcttcatctacggcaacagtgcccgggcctacccgctggcggtggttgtcccgtccggggacgcgctttctcgccatggcatcgagaatctcaagcccgtgatcagcgagtccctgcaggaggtagcgagggcggccggcctgcaatcctacgagattccacgcgacttcatcatcgaaaccacgccgttcaccctggagaacggcctgctcaccggcatccgcaagctggcacgcccgcagttgaagaagttctatggcgaacgtctcgagcggctctataccgagctggccgatagccaatccaacgagctgcgcgagctgcggcaaagcggtcccgatgcgccggtgcttccgacgctgtgccgtgccgcggctgcgttgctgggctctaccgctgcggatgtgcggccggacgcgcacttcgccgacctgggtggtgactcgctctcggcgctgtcgttggccaacctgctgcacgagatcttcggcgtcgacgtgccggtgggtgtcattgtcagcccggcaagcgacctgcgggccctggccgaccacatcgaagcagcgcgcaccggcgtcaggcgacccagcttcgcctcgatacacggtcgctccgcgacggaagtgcacgccagcgacctcacgctggacaagttcatcgacgctgccaccctggccgcagccccgaacctgccggcaccgagcgcccaagtgcgcaccgtactgctgaccggcgccaccggctttttgggtcgctacctggcgctggaatggctcgaccgcatggacctggtcaacggcaagctgatctgcctggtccgcgccagatccgacgaggaagcacaagcccggctggacgcgacgttcgatagcggcgacccgtatttggtgcggcactaccgcgaattgggcgccggccgcctcgaggtgctcgccggcgacaagggcgaggccgacctgggcctggaccgggtcacctggcagcggctagccgacacggtggacctgatcgtggaccccgcggccctggtcaaccacgtgctgccgtatagccagctgttcggcccaaacgcggcgggcaccgccgagttgcttcggctggcgctgaccggcaagcgcaagccatacatctacacctcgacgatcgccgtgggcgagcagatcccgccggaggcgttcaccgaggacgccgacatccgggccatcagcccgacccgcaggatcgacgacagctacgccaacggctacgcgaacagcaagtgggccggcgaggtgctgctgcgcgaagctcacgagcagtgcggcctgccggtgacggtcttccgctgcgacatgatcctggccgacaccagctataccggtcagctcaacctgccggacatgttcacccggctgatgctgagcctggccgctaccggcatcgcacccggttcgttctatgagctggatgcgcacggcaatcggcaacgcgcccactatgacggcttgccggtcgaattcgtcgcagaagccatttgcacccttgggacacatagcccggaccgttttgtcacctaccacgtgatgaacccctacgacgacggcatcgggctggacgagttcgtcgactggctcaactccccaactagcgggtccggttgcacgatccagcggatcgccgactacggcgagtggctgcagcggttcgagacttcgctgcgtgccttgccggatcgccagcgccacgcctcgctgctgcccttgctgcacaactaccgagagcctgcaaagccgatatgcgggtcaatcgcgcccaccgaccagttccgcgctgccgtccaagaagcgaaaatcggtccggacaaagacattccgcacctcacggcggcgatcatcgcgaagtacatcagcaacctgcgactgctcgggctgctgtga

SEQ ID NO: 21 - protein, 1168 aa, Mycobacterium tuberculosis
MSINDQRLTRRVEDLYASDAQFAAASPNEAITQAIDQPGVALPQLIRMVMEGYADRPALGQRALRFVTDPDSGRTMVELLPRFETITYRELWARAGTLATALSAEPAIRPGDRVCVLGFNSVDYTTIDIALIRLGAVSVPLQTSAPVTGLRPIVTETEPTMIATSIDNLGDAVEVLAGHAPARLVVFDYHGKVDTHREAVEAARARLAGSVTIDTLAELIERGRALPATPIADSADDALALLIYTSGSTGAPKGAMYRESQVMSFWRKSSGWFEPSGYPSITLNFMPMSHVGGRQVLYGTLSNGGTAYFVAKSDLSTLFEDLALVRPTELCFVPRIWDMVFAEFHSEVDRRLVDGADRAALEAQVKAELRENVLGGRFVMALTGSAPISAEMTAWVESLLADVHLVEGYGSTEAGMVLNDGMVRRPAVIDYKLVDVPELGYFGTDQPYPRGELLVKTQTMFPGYYQRPDVTAEVFDPDGFYRTGDIMAKVGPDQFVYLDRRNNVLKLSQGEFIAVSKLEAVFGDSPLVRQIFIYGNSARAYPLAVVVPSGDALSRHGIENLKPVISESLQEVARAAGLQSYEIPRDFIIETTPFTLENGLLTGIRKLARPQLKKFYGERLERLYTELADSQSNELRELRQSGPDAPVLPTLCRAAAALLGSTAADVRPDAHFADLGGDSLSALSLANLLHEIFGVDVPVGVIVSPASDLRALADHIEAARTGVRRPSFASIHGRSATEVHASDLTLDKFIDAATLAAAPNLPAPSAQVRTVLLTGATGFLGRYLALEWLDRMDLVNGKLICLVRARSDEEAQARLDATFDSGDPYLVRHYRELGAGRLEVLAGDKGEADLGLDRVTWQRLADTVDLIVDPAALVNHVLPYSQLFGPNAAGTAELLRLALTGKRKPYIYTSTIAVGEQIPPEAFTEDADIRAISPTRRIDDSYANGYANSKWAGEVLLREAHEQCGLPVTVFRCDMILADTSYTGQLNLPDMFTRLMLSLAATGIAPGSFYELDAHGNRQRAHYDGLPVEFVAEAICTLGTHSPDRFVTYHVMNPYDDGIGLDEFVDWLNSPTSGSGCTIQRIADYGEWLQRFETSLRALPDRQRHASLLPLLHNYREPAKPICGSIAPTDQFRAAVQEAKIGPDKDIPHLTAAIIAKYISNLRLLGLL

SEQ ID NO: 22 - DNA, 3507 bp, Mycobacterium smegmatis
ttacagcaatccgagcatctgcaggttgctgatgtacttgacgatcacgtcggccgtgacgtgcggaatgtccttgtcggggccgatcttcgcgtcctgcaccgcggcacggaaccggtcggtgggtgccatggcaccgcacacgggcggtgagggctgctgatagttgtgcagcagcggcagcagcgaggcctgacgttgccgttccggcagggcccgcagtgcggtttcgaaccggctcagccaggtggcgtagtcgtcgacgcggtgcacggggtagccggcctcgatcagccagtccacgtactcgtcgaggccgatgccgtcgtcgtacgggttcatcacgtggaacgtctcgaatccgtcggtgacctgcgagccgatggtggagatcgcctcggcgatgaactccacgggcagcccgtcgtagtgggcgcgctgccggttgccgtccgcatcgagttcgtagaacgaaccgggcgcgatgccggtcgccacgaggctcagcatcaggcgggtgaacatgtccggcaggttcagctgacccgagtaggtcgtgtcggccaggatcatgtcgcagcggaacaccgagaccggcagaccacaccagtcgtgcgcctcccgcagcaggacctcgccggcccacttgctgttgccgtagccgttggcgtacgagtcgtcgacccggcgcgtcgcgctgatctcgcggatgtcggcgtcctcgacgaacgcctcgggggagatgccctgtcccacaccgatcgtcgagacgtacacgtacggcttgatcgtggtggtcagcgcgatccggatgagttcggcggtgccgagcgcattgggtccgaacatctggctgtacggcaggacgtgattgaccagggcggccggatcgacgatcagatcgacggtgtcggccagtcgctgccacgtgtcgtggtcgagacccagatcggcctcgcccttgtcaccggcgatcacctcgaggtgatcggctgccagcgcgcggtagtgctcgagcagtgtcgcgtccccggtgtcgaacgtggcgtccagacgcgcccgggcctcgtcgtcgctgcgggcgcgcaccaggcagatcaccttgccgtccaccaggtccatgcgctccagccattccagcgccagatagcggcccaggaacccggtggcgccggtcagcagcacggtgcggatctcggtgcccgaacgcggcagacccggcgcggcggacagggtcttggcgtcgatgaacttgcccagggcgagatcacgcgcgcgcacctcggtggcgtcgcgcccgtgcaccgacgcgtatgtggggcgcttggagccgcgcagttcgccctcgatgtaggccgcgacgcctgccaggtcggtggccgggctgacgatgacgccgaccggcacgtcgacatcgaagatctcgtgcaacaggttcgagaagctcaaggccgacaacgaatctccacccagatcggtgaagtgcgcatcggaccgcagatccgtgacggaggcaccgagcagtgcgaccgcggcgcggctgacggtctcgaccacgggccggtcggctccgttgcggcgcaactcgcgcaactcgttggcctgcccctcggccaggtcggtgtagagctgttcgaggcgttcgccgtagtgcgccttcagtttcggccgggccagcttgcggataccggtcagcaggccgttctccagcgtgaaaggtgttgtctcgacgaggaagtcacgcgggatctcatacgactgcaatccggcggctcgtgccgcgtcctgcagtgagtcgctgatgcgcgacttgagttcgtcaccgtcccaacgtgacagtgcctcttcggtcgggaccacgaccgccagcagataggaccgcgcgctgttgccgtagacgtagatctggcgtaccagggggctgtcgccgaacaccgcctccagcttggagaccgtgacgaattcgccctgcgacagtttcagcacgttgttgcggcggtcgaggtattcgagatggtcgggcccgagctcggcgacgatgtcgccggtgcggtagtacccgtcctcgtcgaacatctcggcggtgatctccggacgcttgtagtagccggggaacatctgctcggacttgaccagaagttcgccgcgcgggtagggccggtccgtggcgaagtagccgagatcgggcacgtcgaccagcttgtagtcgatgaccggcgggcgctggatctgcccgtcgatgaacaccgcgccggcctcggtggagccgtagccctccagcagatgcatgtcgagcaggtcctcgacccagctcttcatctccgccgagatgggagccgatccggtcagggccgaaacgaatcgcccgccgagcagttgggtgcggacctcttcgaggactgcggcttcggctcggtcctcggatccctcggcgcggcggttgtcgaggcggctctggtactcctggaacagcatgtcccagatgcgaggaacgaagttgagctgcgtgggccgcacgagggcgaggtcctccaggaaggtggacaggtcgctgcgtgcggcgaagtacgcggttccgccgctggcgagtgtgctgcacaggatgccgcgccccatgacgtgactcatgggcatgaagttcagggtgatcgacggcatcacgccgagggtctcgtcccaccgggccttggacccggcctgccacatcgtggcggtcttggactcggggtacatcgcgcccttgggagtgccggtgctgccggaggtgtagatgagaagggtcagcgggtcggcctcgtcgggcacgtagagcggtgcgtcggcgagtgaccgcccgcggtccagtgcgtcggtgatcgtctcgacgacgacgccggtgcctgcgagcttgcccttggccgcctcgaacgcctcacgctgatcgtcgacctcgtggctgtagtcgaacaccaccagtcgcgacggcgcgggcccggactcgacgagagcgactgcgtcggcgaggaagtcgacgctcgacgcgatcaccttgggctcggtctcggcgacgatcggctgcagttgggccaccggcgcactggtctgcagcggtacggacacggcgccgagttcgagcagggcgatgtcgatcgtcgtgtagtcgacactggtgaaacccaggatggccacgcggtcaccggcattcaccggatggttgtgccaggcattggtcacggcctggatccggcctgcgagctgacggtaggtgatggtgtcgaagcggggcaggagcttcgcggtggtgcggccttcttcgtcggtgacgaactcgacggcgcgcttgcccagcgcagggcggtccgcatagccggccagaatctgtttgaccgcggcaggaaggcgcaactccggatcggcggcagccgcgctgatcgcctcgtcgggacgggcggcggcgaactgcgggtcggtttcgaacaagtggtcaatgcgccggttgaagcggtcttcgcgcgtttcgatcgtcat

SEQ ID NO: 23 - protein, 1168 aa, Mycobacterium smegmatis
MTIETREDRFNRRIDHLFETDPQFAAARPDEAISAAAADPELRLPAAVKQILAGYADRPALGKRAVEFVTDEEGRTTAKLLPRFDTITYRQLAGRIQAVTNAWHNHPVNAGDRVAILGFTSVDYTTIDIALLELGAVSVPLQTSAPVAQLQPIVAETEPKVIASSVDFLADAVALVESGPAPSRLVVFDYSHEVDDQREAFEAAKGKLAGTGVVVETITDALDRGRSLADAPLYVPDEADPLTLLIYTSGSTGTPKGAMYPESKTATMWQAGSKARWDETLGVMPSITLNFMPMSHVMGRGILCSTLASGGTAYFAARSDLSTFLEDLALVRPTQLNFVPRIWDMLFQEYQSRLDNRRAEGSEDRAEAAVLEEVRTQLLGGRFVSALTGSAPISAEMKSWVEDLLDMHLLEGYGSTEAGAVFIDGQIQRPPVIDYKLVDVPDLGYFATDRPYPRGELLVKSEQMFPGYYKRPEITAEMFDEDGYYRTGDIVAELGPDHLEYLDRRNNVLKLSQGEFVTVSKLEAVFGDSPLVRQIYVYGNSARSYLLAVVVPTEEALSRWDGDELKSRISDSLQDAARAAGLQSYEIPRDFLVETTPFTLENGLLTGIRKLARPKLKAHYGERLEQLYTDLAEGQANELRELRRNGADRPVVETVSRAAVALLGASVTDLRSDAHFTDLGGDSLSALSFSNLLHEIFDVDVPVGVIVSPATDLAGVAAYIEGELRGSKRPTYASVHGRDATEVRARDLALGKFIDAKTLSAAPGLPRSGTEIRTVLLTGATGFLGRYLALEWLERMDLVDGKVICLVRARSDDEARARLDATFDTGDATLLEHYRALAADHLEVIAGDKGEADLGLDHDTWQRLADTVDLIVDPAALVNHVLPYSQMFGPNALGTAELIRIALTTTIKPYVYVSTIGVGQGISPEAFVEDADIREISATRRVDDSYANGYGNSKWAGEVLLREAHDWCGLPVSVFRCDMILADTTYSGQLNLPDMFTRLMLSLVATGIAPGSFYELDADGNRQRAHYDGLPVEFIAEAISTIGSQVTDGFETFHVMNPYDDGIGLDEYVDWLIEAGYPVHRVDDYATWLSRFETALRALPERQRQASLLPLLHNYQQPSPPVCGAMAPTDRFRAAVQDAKIGPDKDIPHVTADVIVKYISNLQMLGLL

(19) United States
(12) Patent Application Publication
(10) Pub. No.: US 2015/0125933 A1
(43) Pub. Date: May 7, 2015

(54) PRODUCTION OF FATTY ACIDS AND DERIVATIVES THEREOF HAVING IMPROVED ALIPHATIC CHAIN LENGTH AND SATURATION CHARACTERISTICS

(71) Applicants: Eli S. Groban, San Francisco, CA (US); Vikranth Arlagadda, South San Francisco, CA (US); Scott A. Frykman, (US); Derek L. Greenfield, South San Francisco, CA (US); Zhihao Hu, South San Francisco, CA (US)

(72) Inventors: Eli S. Groban, San Francisco, CA (US); Vikranth Arlagadda, South San Francisco, CA (US); Scott A. Frykman, (US); Derek L. Greenfield, South San Francisco, CA (US); Zhihao Hu, South San Francisco, CA (US)

(73) Assignee: LS9, INC., South San Franciso, CA (US)

(21) Appl. No.: 14/236,883

(22) PCT Filed: Aug. 17, 2012
(86) PCT No.: PCT/US12/48620

§ 371 Date: May 22, 2014

Related U.S. Application Data
(60) Provisional application No. 61/514,861, filed on Aug. 3, 2011.

(51) Int. Cl. C12N 15/70; C12N 9/02; C12N 9/20; C12N 9/00; C12N 9/88; C12N 9/10

(52) U.S. Cl. CPC C12N 15/70; C12N 9/88; C12N 9/1029; C12N 9/20; C12N 9/93; C12N 9/0008; C12Y 402/0106; C12Y 301/01005; C12Y 602/01003; C12Y 102/01042
USPC 435/252.33

(57) ABSTRACT

The invention relates to compositions, including polynucleotide sequences, amino acid sequences, recombinant microorganisms, and recombinant microorganism cultures that produce compositions of fatty acids and derivatives having target aliphatic chain lengths and/or preferred percent saturation. Further, the invention relates to methods of making and using the compositions. The compositions and methods provide for high titers, high yields, and high productivities of fatty acids and derivatives thereof.

This application claims priority benefit to U.S. Provisional Application Ser. No. 61/514,861, filed on Aug. 3, 2011, which is expressly incorporated by reference herein in its entirety.

### FIELD OF THE INVENTION

The invention relates to methods for producing and compositions of fatty acids and derivatives thereof having selected aliphatic chain lengths and/or saturation characteristics. Further, the invention relates to recombinant host cells (e.g., microorganisms), cultures of recombinant host cells, and methods of making and using recombinant host cells, for example, using cultures of the recombinant host cells in the fermentative production of fatty acids and derivatives thereof having selected aliphatic chain lengths and saturation characteristics.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Jul. 27, 2012, is named LS0036PCT.txt and is 74,934 bytes in size

### BACKGROUND OF THE INVENTION

The biosynthesis of fatty acids in most living organisms involves the action of a series of enzymes on acetyl-CoA and malonyl-CoA precursors. Two important cofactors in fatty acid biosynthesis are coenzyme A (CoA) and acyl carrier protein (ACP). These two cofactors are involved in carrying the growing acyl chain from one enzyme to another and supplying precursors for the condensation reactions.

The fatty acid biosynthetic cycle in Escherichia coli (E. coli) provides a convenient frame of reference for discussion of this cycle. Heath, R. J., et al., (J Biol. Chem. 271(44):27795-801 (1996)) provide an overview of E. coli fatty acid biosynthesis. The malonyl-ACP used by the condensing enzymes is produced by the transacylation of malonyl-CoA to malonyl-ACP, which is catalyzed by malonyl-CoA:ACP transacylase (fabD). In each cycle of fatty acid elongation there are basically four reactions. The cycle is initiated by β-ketoacyl-ACP synthase III (fabH) condensing malonyl-ACP with acetyl-CoA.

The following description of the elongation cycle is given with reference to FIG. 1. Elongation cycles begin with the condensation of malonyl-ACP and an acyl-ACP catalyzed by β-ketoacyl-ACP synthase I (fabB) and β-ketoacyl-ACP synthase II (fabF) to produce a β-keto-acyl-ACP.

Second, the β-keto-acyl-ACP is reduced by a NADPH-dependent β-ketoacyl-ACP reductase (fabG) to produce a β-hydroxy-acyl-ACP.

Third, β-hydroxy-acyl-ACP is dehydrated to a trans-2-enoyl-acyl-ACP by either the fabA or fabZ β-hydroxyacyl-ACP dehydratase. FabA can also isomerize trans-2-enoyl-acyl-ACP to cis-3-enoyl-acyl-ACP, which can bypasses fabI and can used by fabB (typically for up to an aliphatic chain length of C16) to produce β-keto-acyl-ACP.

The fourth step in each cycle is catalyzed by a NADH or NADHPH-dependent enoyl-ACP reductase (fabI) that converts trans-2-enoyl-acyl-ACP to acyl-ACP.

In the methods described herein, termination of fatty acid synthesis occurs by thioesterase removal of the acyl group from acyl-ACP to release free fatty acids (FFA). Thioesterases (e.g., tesA) hydrolyze thioester bonds, which occur between acyl chains and ACP through sulfydryl bonds.

### SUMMARY OF THE INVENTION

The present invention generally relates to recombinant host cells, cultures of recombinant host cells, methods of making recombinant host cells, and methods of using recombinant host cells that produce a wide range of aliphatic chain lengths of fatty acid derivatives from which recombinant host cells producing specific fatty acid derivatives are obtained. The present invention provides one of ordinary skill in the art the ability to select recombinant host cells that produce fatty acid derivatives with desired target aliphatic chain lengths and desired levels of saturation. The methods, recombinant microorganisms and cultures of the present invention can be used in methods to produce fatty acid derivatives at titers, yields, and productivities greater than the titers, yields, and productivities reported prior to the present invention.

In a first aspect, the present invention relates to recombinant host cell cultures engineered to produce a high titer fatty acid derivative composition having target aliphatic chain lengths, the high titer typically being between about 30 g/L to about 250 g/L.

In embodiments of the recombinant host cells of the present invention, the polynucleotide sequences comprise an open reading frame encoding an elongation β-ketoacyl-ACP synthase protein having an Enzyme Commission number of EC 2.3.1.-. The coding sequences are operably-linked to regulatory sequences that facilitate expression of the protein in recombinant host cells. The activity of the β-ketoacyl-ACP synthase protein in the recombinant host cell is modified relative to the activity of the β-ketoacyl-ACP synthase protein expressed from the wild-type gene in a corresponding host cell. Additionally, the recombinant host cells in the culture comprise one or more polynucleotide sequences that comprise an open reading frame encoding a thioesterase, having an Enzyme Commission number of EC 3.1.1.5 or EC 3.1.2.-. The coding sequences are operably-linked to regulatory sequences that facilitate expression of the protein in recombinant host cells. The activity of the thioesterase in the recombinant host cell is modified relative to the activity of the thioesterase expressed from the corresponding wild-type gene in a corresponding host cell.

A recombinant culture of the present invention typically produces a higher titer, higher yield, and/or higher productivity of fatty acid derivatives having target aliphatic chain length and preferred percent saturation as compared to control cultures.

The recombinant host cells and host cell cultures of the present invention can further comprise one or more nucleotide sequence encoding a carboxylic acid reductase protein that has an Enzyme Commission number of EC 6.2.1.3 or EC 1.2.1.42, and operably-linked regulatory sequences.

A second aspect of the present invention relates to providing a desired degree of saturation of the aliphatic chains of the fatty acid derivatives (e.g., fatty alcohols). In this aspect, the recombinant host cells of the present invention further comprise one or more polynucleotide sequences that comprise an open reading frame encoding a β-hydroxyacyl-ACP dehydratase protein, having an Enzyme Commission number of EC 4.2.1.- or 4.2.1.60, and operably-linked regulatory sequences. The activity of the β-hydroxyacyl-ACP dehydratase protein in the recombinant host cell is modified relative to the activity of the β-hydroxyacyl-ACP dehydratase protein expressed from the wild-type gene in a corresponding host cell.

A third aspect of the present invention relates to recombinant host cell cultures that produce compositions of fatty acid derivatives having target aliphatic chain lengths. The recombinant host cells typically have a modified activity of a β-hydroxyacyl-ACP dehydratase protein, having an Enzyme Commission number of EC 4.2.1.- or 4.2.1.60. The activity of the β-hydroxyacyl-ACP dehydratase protein in the recombinant host cell is modified relative to the activity of the β-hydroxyacyl-ACP dehydratase protein expressed from the wild-type gene in a corresponding host cell.

A fourth aspect the present invention relates to recombinant host cell cultures that produce compositions of fatty acid derivatives having preferred percent saturation. The recombinant host cells comprise a modified activity of a β-hydroxyacyl-ACP dehydratase protein that lacks isomerase activity, having an Enzyme Commission number of EC 4.2.1.-. The activity of the β-hydroxyacyl-ACP dehydratase protein that lacks isomerase activity in the recombinant host cell is modified relative to the activity of the β-hydroxyacyl-ACP dehydratase protein that lacks isomerase activity expressed from the wild-type gene in a corresponding host cell.

In the recombinant host cell cultures of the present invention, the recombinant host cell can be a mammalian cell, plant cell, insect cell, fungus cell, algal cell or a bacterial cell.

Embodiments of the recombinant host cells of the cultures of present invention can further comprise one or more nucleotide sequence encoding one or more additional proteins and operably-linked regulatory sequences. Examples of such additional proteins include, but are not limited to, a carboxylic acid reductase protein, having an Enzyme Commission number of EC 6.2.1.3 or EC 1.2.1.42, and an alcohol dehydrogenase protein, having an Enzyme Commission number of EC 1.1.-.-, EC 1.1.1.1, or EC 1.2.1.10. Such additional proteins can be expressed in the recombinant host cells to facilitate production of particular fatty acid derivatives from acyl-ACPs as substrates.

A fifth aspect of the present invention relates to methods of making the recombinant host cells and recombinant host cell cultures of the present invention. Recombinant host cells can be made, by the methods of the present invention, that produce compositions of fatty acid derivatives (e.g., fatty alcohols) having target aliphatic chain lengths. The method generally comprises two core steps selected from the group consisting of step (A), step (B), and step (C). Typically, the two steps are not the same step and the two steps can be performed in any order to make the recombinant host cells; for example, step (A) followed by step (B), step (A) followed by step (C), step (B) followed by step (A), step (B) followed by step (C), step (C) followed by step (B), or step (C) followed by step (A).

Briefly, method step (A) relates to selecting recombinant host cells producing fatty acid derivatives having aliphatic chain lengths longer than the target aliphatic chain length. Method step (B) relates to selecting recombinant host cells producing high titers of fatty acid derivatives having the target aliphatic chain length. Method step (C) relates to selecting recombinant host cells producing a high titer of the fatty acid derivative having the target aliphatic chain length and a preferred percent saturation.

In preferred embodiments of the methods of the, present invention, the recombinant host cell further comprises one or more nucleotide sequence encoding a carboxylic acid reductase protein and operably-linked regulatory sequences. The carboxylic acid reductase protein is typically a protein having an Enzyme Commission number of EC 6.2.1.3 or EC 1.2.1.42.

In further embodiments of the methods of the present invention, the recombinant host cell further comprises one or more nucleotide sequence encoding one or more additional protein and operably-linked regulatory sequences. Examples of such additional proteins include, but are not limited to: alcohol dehydrogenase; aldehyde-alcohol dehydrogenase; acetyl-CoA acetyltransferase; β-hydroxybutyryl-CoA dehydrogenase; crotonase butyryl-CoA dehydryogenase; and coenzyme A-acylating aldehyde dehydrogenase. Such additional proteins can be expressed in the recombinant host cells to facilitate production of particular fatty acid derivatives from acyl-ACPs as substrates.

In a sixth aspect, the present invention relates more specifically to methods of making the recombinant host cells and recombinant host cell cultures that produce compositions of fatty acid derivatives having target aliphatic chain lengths. These recombinant host cells typically have a modified activity of a β-hydroxyacyl-ACP dehydratase protein, having an Enzyme Commission number of EC 4.2.1.- or 4.2.1.60. The methods of the present invention used to make these recombinant host cells typically use at least step (C) or a variation of step (A).

In a seventh aspect the present invention relates more specifically to methods of making the recombinant host cells and recombinant host cell cultures that produce compositions of fatty acid derivatives having preferred percent saturation. These recombinant host cells typically have a modified activity of a β-hydroxyacyl-ACP dehydratase protein that lacks isomerase activity, having an Enzyme Commission number of EC 4.2.1.-. The methods of the present invention used to make these recombinant host cells typically use at least step (C) or a variation of step (A).

In an eighth aspect, the present invention relates more specifically to a method of producing a composition of fatty acid derivatives having a target aliphatic chain length and/or preferred degree of saturation, for example, by culturing, in the presence of a carbon source, a recombinant host cell as described herein. In one embodiment of this method; the culturing comprises fermentation.

In a ninth aspect, the present invention relates to substantially purified compositions of fatty acid derivatives having target aliphatic chain lengths and/or preferred degrees of saturation produced using the recombinant host cell cultures of the present invention.

These and other aspects and embodiments of the present invention will readily occur to those of ordinary skill in the art in view of the disclosure herein.

### BRIEF DESCRIPTION OF THE FIGURES

FIG. 1 presents an overview of an example of a fatty acid biosynthesis pathway with reference to gene products from E. coli.

FIG. 2 presents a schematic view of acyl-ACPs as substrates for enzymes that convert them to fatty acid derivatives.

FIG. 3 presents schematic representations, in panels A through D, of a number of expression constructs used to exemplify embodiments of the present invention.

FIG. 4 presents screening data for clones wherein the activity of the thioesterase in the recombinant microorganism was modified relative to the thioesterase activity in the control microorganism. In the figure, the Y-axis is “% Fatty Species (“FA”=Free Fatty Acid plus Fatty Aldehyde plus Fatty Alcohol) vs. Control Strain,” and the X-axis is the C₁₂/C₁₄ ratio. Each data point in the figure corresponds to a cultured clone or a cultured control strain.

FIG. 5 presents screening data for clones wherein the activity of the thioesterase in the recombinant microorganism was modified relative to the thioesterase activity in the control microorganism. In the figure, the Y-axis is “% FA vs. Control Strain,” and the X-axis is the C₁₆/C₁₈ ratio. Each data point in the figure corresponds to a cultured clone or a cultured control strain.

FIG. 6 presents screening data for clones wherein the activity of the elongation β-ketoacyl-ACP synthase protein in the recombinant microorganism was modified relative to the elongation β-ketoacyl-ACP synthase protein in the control microorganism. In the figure, the Y-axis is “% FA vs. Control Strain,” and the X-axis is the C₁₂/C₁₄ ratio. Each data point in the figure corresponds to a cultured clone or a cultured control strain.

FIG. 7 presents screening data for clones wherein the activity of the elongation β-ketoacyl-ACP synthase protein in the recombinant microorganism was modified relative to the elongation β-ketoacyl-ACP synthase protein in the control microorganism. In the figure, the Y-axis is “% FA vs. Control Strain,” and the X-axis is the C₁₂/C₁₈ ratio. Each data point in the figure corresponds to a cultured clone or a cultured control strain.

FIG. 8 presents screening data for clones wherein the activity of the thioesterase in the recombinant microorganism was modified relative to the thioesterase activity in the control microorganism. In the figure, the Y-axis is “% FA vs. Control Strain,” and the X-axis is the C₁₂/C₁₄ ratio. Each data point in the figure corresponds to a cultured clone or a cultured control strain.

FIG. 9 presents screening data for clones wherein the activity of the thioesterase in the recombinant microorganism was modified relative to the thioesterase activity in the control microorganism. In the figure, the Y-axis is “% FA vs. Control Strain,” and the X-axis is the C₁₆/C₁₈ ratio. Each data point in the figure corresponds to a cultured clone or a cultured control strain.

FIG. 10 presents screening data for clones wherein the activity of an elongation β-ketoacyl-ACP synthase protein in the recombinant microorganisms was modified to evaluate the effect on aliphatic chain length and saturation. In the figure, the left Y-axis is “% Saturated Species”; the right Y-axis is the C₁₂/C₁₄ ratio for titers of fatty acid derivatives (combined free fatty acids and fatty alcohols) having C₁₂ and C₁₄ aliphatic chain lengths. The clones from the screened group of recombinant microorganisms are arranged along the X-axis based on their % Saturated Species and the corresponding data points for their C₁₂/C₁₄ ratios are shown.

FIG. 11 presents screening data for clones wherein the activity of the β-hydroxyacyl-ACP dehydratase protein (here β-hydroxydecanoyl thioester dehydratase/isomerase protein the E. coli fabA protein) in the recombinant microorganisms was modified to evaluate the effect on aliphatic chain length and saturation. In the figure, the left Y-axis is “% Saturated Species”; the right Y-axis is the C₈/C₁₀ ratio for titers of fatty acid derivatives (combined free fatty acids and fatty alcohols) having C₈ and C₁₀ aliphatic chain lengths. The clones from the screened group of recombinant microorganisms are arranged along the X-axis based on their % Saturated Species and the corresponding data points for their C₈/C₁₀ ratios are shown.

FIG. 12 presents screening data for clones wherein the activity of the β-hydroxyacyl-ACP dehydratase protein (here β-hydroxydecanoyl thioester dehydratase/isomerase protein the E. coli fabA protein) in the recombinant microorganisms was modified to evaluate the effect on aliphatic chain length and saturation. In the, figure, the left Y-axis is “% Saturated Species”; the right Y-axis is the C₁₂/C₁₄ ratio for titers of fatty acid derivatives (combined free fatty acids and fatty alcohols) having C₁₂ and C₁₄ aliphatic chain lengths. The clones from the screened group of recombinant microorganisms are arranged along the X-axis based on their % Saturated Species and the corresponding data points for their C₁₂/C₁₄ ratios are shown.

FIG. 13 presents screening data for clones wherein the activity of the β-hydroxyacyl-ACP dehydratase protein (here β-hydroxydecanoyl thioester dehydratase/isomerase protein the E. coli fabA protein) in the recombinant microorganisms was modified to evaluate the effect on aliphatic chain length and saturation. In the figure, the left Y-axis is “% Saturated Species”; the right Y-axis is the C₁₆/C₁₈ ratio for titers of fatty acid derivatives (combined free fatty acids and fatty alcohols) having C₁₆ and C₁₈ aliphatic chain lengths. The clones from the screened group of recombinant microorganisms are arranged along the X-axis based on their % Saturated Species and the corresponding data points for their C₁₆/C₁₈ ratios are shown.

FIG. 14 presents screening data for clones wherein the activity of the β-hydroxyacyl-ACP dehydratase protein (here (3R)-hydroxymyristol acyl carrier protein dehydratase protein, the E. coli fabZ protein) in the recombinant microorganisms was modified to evaluate the effect on aliphatic chain length and saturation. In the figure, the left Y-axis is “% Saturated Species”; the right Y-axis is the C₈/C₁₀ ratio for titers, of fatty acid derivatives (combined free fatty acids and fatty alcohols) having C₈ and C₁₀ aliphatic chain lengths. The clones from the screened group of recombinant microorganisms are arranged along the X-axis based on their % Saturated Species and the corresponding data points for their C₈/C₁₀ ratios are shown.

FIG. 15 presents screening data for clones wherein the activity of the β-hydroxyacyl-ACP dehydratase protein (here (3R)-hydroxymyristol acyl carrier protein dehydratase protein, the E. coli fabZ protein) in the recombinant microorganisms was modified to evaluate the effect on aliphatic chain length and saturation. In the figure, the left Y-axis is “% Saturated Species”; the right Y-axis is the C₁₂/C₁₄ ratio for titers of fatty acid derivatives (combined free fatty acids and fatty alcohols) having C₁₂ and C₁₄ aliphatic chain lengths. The clones from the screened group of recombinant microorganisms are arranged along the X-axis based on their % Saturated Species and the corresponding data points for their C₁₂/C₁₄ ratios are shown.

FIG. 16 presents screening data for clones wherein the activity of the β-hydroxyacyl-ACP dehydratase protein (here (3R)-hydroxymyristol acyl carrier protein dehydratase protein, the E. coli fabZ protein) in the recombinant microorganisms was modified to evaluate the effect on aliphatic chain length and saturation. In the figure, the left Y-axis is “% Saturated Species”; the right Y-axis is the C₁₆/C₁₈ ratio for titers of fatty acid derivatives (combined free fatty acids and fatty alcohols) having C₁₆ and C₁₈ aliphatic chain lengths. The clones from the screened group of recombinant microorganisms are arranged along the X-axis based on their % Saturated Species and the corresponding data points for their C₁₆/C₁₈ ratios are shown.

FIG. 17 presents screening data for strains wherein the activity of the β-hydroxyacyl-ACP dehydratase protein (here β-hydroxydecanoyl thioester dehydratase/isomerase protein the E. coli fabA protein) in the recombinant microorganisms was modified to evaluate the effect on aliphatic chain length and saturation. In the figure, the left Y-axis is “% Saturated Species”; the right Y-axis is the C₁₂/C₁₄ ratio for titers of fatty acid derivatives (combined free fatty acids and fatty alcohols) having C₁₂ and C₁₄ aliphatic chain lengths. Two strains are indicated at the bottom of the figure on the X-axis: “ALC487” and “D178 PT5_fabA/pALC487.” In the figure, for each of the two strains, the C₁₂/C₁₄ ratio is indicated by a diamond and the % Saturated Species is indicated by the bar graph.

FIG. 18 presents screening data for strains wherein the activity of the β-hydroxyacyl-ACP dehydratase protein (here β-hydroxydecanoyl thioester dehydratase/isomerase protein the E. coli fabA protein) in the recombinant microorganisms was modified to evaluate the effect on aliphatic chain length and saturation. In the figure, the left Y-axis is “% Saturated Species”; the right Y-axis is the C₈/C₁₀ ratio for titers of fatty acid derivatives (combined free fatty acids and fatty alcohols) having C₈ and C₁₀ aliphatic chain lengths. Two strains are indicated at the bottom of the figure on the X-axis: “ALC487” and “D178 PT5_fabA/pALC487.” In the figure, for each of the two strains, the C₈/C₁₀ ratio is indicated by a diamond and the % Saturated Species is indicated by the bar graph.

FIG. 19 presents screening data for strains wherein the activity of the β-hydroxyacyl-ACP dehydratase protein (here β-hydroxydecanoyl thioester dehydratase/isomerase protein the E. coli fabA protein) in the recombinant microorganisms was modified to evaluate the effect on aliphatic chain length and saturation. In the figure, the left Y-axis is “% Saturated Species”; the right Y-axis is the C₁₆/C₁₈ ratio for titers of fatty acid derivatives (combined free fatty acids and fatty alcohols) having C₁₆ and C₁₈ aliphatic chain lengths. Two strains are indicated at the bottom of the figure on the X-axis: “ALC487” and “D178 PT5_fabA/pALC487.” In the figure, for each of the two strains, the C₁₆/C₁₈ ratio is indicated by a diamond and the % Saturated Species is indicated by the bar graph.

FIGS. 20A-B present the chain length distribution for fatty species (“FAS”; fatty alcohol and free fatty acid) production at 55 hours from fatty alcohol production strains modified by addition of FabB to the carB operon. Data is presented for the parent strain (Alc-287; FIG. 20A) and a variant with an additional copy of fabB expressed in the cells (Alc-383; FIG. 20B).

FIGS. 21A-D present the chain length distribution for fatty species (“FAS”; fatty alcohol and free fatty acid) production at 58 hours from fatty alcohol production strains modified by addition of FabA to the carB operon. Data is presented for the parent strain (LC-302; FIG. 21A) and three variants with differing amounts of fabA expressed in the cells (LC-369; FIG. 21B, LC-372; FIG. 21C, LC-375; FIG. 21D).

### DETAILED DESCRIPTION OF THE INVENTION

All patents, publications, and patent applications cited in this specification are herein incorporated by reference as if each individual patent, publication, or patent application was specifically and individually indicated to be incorporated by reference in its entirety for all purposes.

### Definitions

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a recombinant microorganism” includes two or more such recombinant microorganisms, reference to “a fatty acid derivative” includes one or more fatty acid derivative, or mixtures of fatty acids derivatives, reference to “a polynucleotide sequence” includes one or more polynucleotide sequences, reference to “an enzyme” includes one or more enzymes, reference to “a control sequence” includes one or more control sequences, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

As used herein, the term “nucleotide” refers to a monomeric unit of a polynucleotide that consists of a heterocyclic base, a sugar, and one or more phosphate groups. The naturally occurring bases (guanine, (G), adenine, (A), cytosine, (C), thymine, (T), and uracil (U)) are typically derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleic acids are typically linked via phosphate bonds to form nucleic acids or polynucleotides, though many other linkages are known in the art (e.g., phosphorothioates, boranophosphates, and the like).

As used herein, the term “polynucleotide” refers to a polymer of ribonucleotides (RNA) or deoxyribonucleotides (DNA), which can be single-stranded or double-stranded and which can contain non-natural or altered nucleotides. The terms “polynucleotide,” “nucleic acid sequence,” and “nucleotide sequence” are used interchangeably herein to refer to a polymeric form of nucleotides of any length, either RNA or DNA. These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides. The polynucleotide can be in any form, including but not limited to, plasmid, viral, chromosomal, EST, cDNA, mRNA, and rRNA.

As used herein, the terms “polypeptide” and “protein” are used interchangeably to refer to a polymer of amino acid residues. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide.

As used herein, the terms “homolog,” and “homologous” refer to a polynucleotide or a polypeptide comprising a sequence that is at least about 50% identical to the corresponding polynucleotide or polypeptide sequence. Preferably homologous polynucleotides or polypeptides have polynucleotide sequences or amino acid sequences that have at least about 80%, at least about 85%, at least about 90%, at least about 91%, at least about 92%, at least about 93%, at least about 94%, at least about 95%, at least about 96%, at least about 97%, at least about 98%, or at least about 99% homology to the corresponding amino acid sequence or polynucleotide sequence. As used herein the terms sequence “homology” and sequence “identity” are used interchangeably.

One of ordinary skill in the art is well aware of methods to determine homology between two or more sequences. Briefly, calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a first sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of a second sequence. The amino acid residues or nucleotides at corresponding amino acid positions or nucleotide positions of the first and second sequences are then compared. When a position in the first sequence is occupied by the same amino acid residue or nucleotide as the corresponding position in the second sequence, then the molecules are identical at that position. The percent homology between the two sequences is a function of the number of identical positions shared by the sequences, taking into account the number of gaps and the length of each gap, that need to be introduced for optimal alignment of the two sequences.

The comparison of sequences and determination of percent homology between two sequences can be accomplished using a mathematical algorithm, such as BLAST (Altschul, et al., J. Mol. Biol., 215(3): 403-410 (1990)). The percent homology between two amino acid sequences also can be determined using the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3,4, 5, or 6 (Needleman and Wunsch, J. Mol. Biol., 48: 444-453 (1970)). The percent homology between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial homology calculations and adjust the algorithm parameters accordingly. A preferred set of parameters (and the one that should be used if a practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5. Additional methods of sequence alignment are known in the biotechnology arts (see, e.g., Rosenberg, BMC Bioinformatics, 6: 278 (2005); Altschul, et al., FEBS J., 272(20): 5101-5109 (2005)).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and non-aqueous methods are described in that reference and either method can be used. Specific hybridization conditions referred to herein are as follows: 1) low stringency hybridization conditions—6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions—6×SSC at about 45° C., followed by one or more washes in 0.2×SSC, 0.1% SDS at 60° C.; 3) high stringency hybridization conditions—6×SSC at about 45° C., followed by one or more washes in 0.2.×SSC, 0.1% SDS at 65° C.; and 4) very high stringency hybridization conditions—0.5M sodium phosphate, 7% SDS at 65° C., followed by one or more washes at 0.2×SSC, 1% SDS at 65° C. Very high stringency conditions (4) are the preferred conditions unless otherwise specified.

The term “heterologous” as used herein typically refers to a nucleotide sequence or a protein not naturally present in an organism. For example, a polynucleotide sequence endogenous to a plant can be introduced into a bacterial cell by recombinant methods, and the plant polynucleotide is then a heterologous polynucleotide in the bacterial cell.

As used herein, the term “fragment” of a polypeptide refers to a shorter portion of a full-length polypeptide or protein ranging in size from four amino acid residues to the entire amino acid sequence minus one amino acid residue. In certain embodiments of the invention, a fragment refers to the entire amino acid sequence of a domain of a polypeptide or protein (e.g., a substrate binding domain or a catalytic domain).

As used herein, the terms “mutant” and “variant” polypeptide are used interchangeably herein to refer to a polypeptide having an amino acid sequence that differs from the corresponding wild-type polypeptide by at least one amino acid. In some: embodiments, the mutant polypeptide has about 1, 2, 3, 4, 5, 6, 7, 8, 9, 10, 15, 20, 30, 40, 50, 60, 70, 80, 90, 100, or more amino acid substitutions, additions, insertions, or deletions. For example, the mutant can comprise one or more conservative amino acid substitutions. As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

Preferred variants of a polypeptide or fragments a polypeptide retain some or all of the biological function (e.g., enzymatic activity) of the corresponding wild-type polypeptide. In some embodiments, the variant or fragment retains at least about 75% (e.g., at least about 80%, at least about 90%, or at least about 95%) of the biological function of the corresponding wild-type polypeptide. In other embodiments, the variant or fragment retains about 100% of the biological function of the corresponding wild-type polypeptide. In still further embodiments, the variant or fragment has greater than 100% of the biological function of the corresponding wild-type polypeptide. Guidance in determining which amino acid residues may be substituted, inserted, or deleted without affecting biological activity may be found using computer programs well known in the art, for example, LASERGENE™ software (DNASTAR, Inc., Madison, Wis.).

It is understood that the polypeptides described herein may have additional conservative or non-essential amino acid substitutions, which do not have a substantial effect on the polypeptide function. Whether or not a particular substitution will be tolerated (i.e., will not adversely affect the desired biological function, such as carboxylic acid reductase activity or thioesterase activity) can be determined as described in Bowie, et al. (Science, 247: 1306-1310 (1990)).

As used herein “an open reading frame derived from a wild-type gene” encoding a protein includes, but is not limited to, the following: an open reading frame that encodes the wild-type protein encoded by the gene; an open reading frame that encodes a variant of the wild-type protein encoded by the gene (e.g., a variant protein having a different sequence obtained, for example, by modification of the wild-type: protein); and, an open reading frame that encodes the wild-type protein wherein the open reading frame is codon optimized. Some examples of open reading frames derived from wild-type genes are illustrated herein (see, e.g., an optimized nucleotide sequence (SEQ ID NO:15) of wild-type, Mycobacterium smegmatis carB, fatty acid reductase protein; a variant protein coding sequence derived from the E. coli tesA (12H08: SEQ ID NO:18), thioesterase protein).

As used herein, the term “mutagenesis” refers to a process by which the genetic information of an organism is changed in a stable manner. Mutagenesis of a protein coding nucleic acid sequence produces a mutant protein. Mutagenesis also refers to changes in non-coding nucleic acid sequences that result in modified protein activity.

As used herein, the term “gene” refers to nucleic acid sequences encoding either an RNA product or a protein product, as well as operably-linked nucleic acid sequences affecting the expression of the RNA or protein (e.g., such sequences include but are not limited to promoter or enhancer sequences) or operably-linked nucleic acid sequences encoding sequences that affect the expression of the RNA or protein (e.g., such sequences include but are not limited to ribosome binding sites or translational control sequences).

As used herein “Acyl-CoA” refers to an acyl thioester formed between the carbonyl carbon of alkyl chain and the sulfydryl group of the 4′-phosphopantethionyl moiety of coenzyme A (CoA), which has the formula R—C(O)S—CoA, where R is any alkyl group having at least 4 carbon atoms.

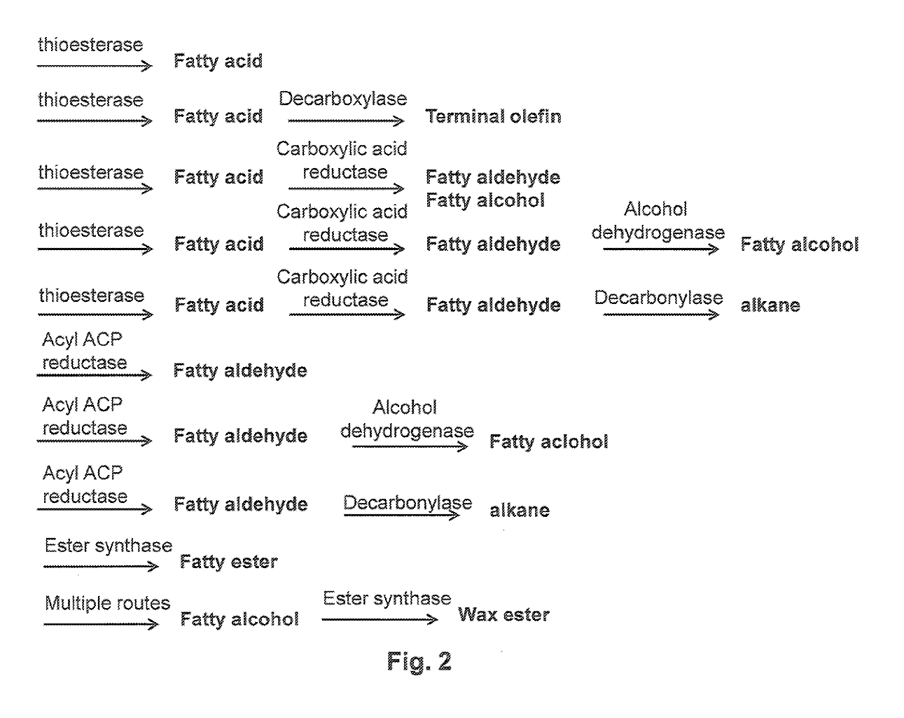

As used herein “Acyl-ACP” refers to an acyl thioester formed between the carbonyl carbon of alkyl chain and the sulfydryl group of the phosphopantetheinyl moiety of an acyl carrier protein (ACP). The phosphopantetheinyl moiety is post-translationally attached to a conserved serine residue on the ACP by the action of holo-acyl carrier protein synthase (ACPS), a phosphopantetheinyl transferase. In some embodiments an acyl-ACP is an intermediate in the synthesis of fully saturated acyl-ACPs. In other embodiments an acyl-ACP is an intermediate in the synthesis of unsaturated acyl-ACPs. In some embodiments, the carbon chain will have about 5, 6, 7, 8, 9, 10, 11, 12, 13, 14, 15, 16, 17, 18, 19, 20, 21, 22, 23, 24, 25, or 26 carbons. Each of these acyl-ACPs are substrates for enzymes that convert them to fatty acid derivatives such as those described in FIG. 2.

As used herein, “fatty aldehyde” means an aldehyde having the formula RCHO characterized by a carbonyl group (C═O). In some embodiments, the fatty aldehyde is any aldehyde made from a fatty acid or fatty acid derivative. In certain embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty aldehyde is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty aldehyde. In certain embodiments, the fatty aldehyde is a C₆, C₈, C₁₀, C₁₂, C₁₃, C₁₄, C₁₅, C₁₆, C₁₇, or C₁₈ fatty aldehyde.

As used herein, “fatty alcohol” means an alcohol having the formula ROH. In some embodiments, the fatty alcohol is any alcohol made from a fatty acid or fatty acid derivative. In certain embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty alcohol is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty alcohol. In certain embodiments, the fatty alcohol is a C₆, C₈, C₁₀, C₁₂, C₁₃, C₁₄, C₁₅, C₁₆, C₁₇, or C₁₈ fatty alcohol. A microorganism engineered to produce fatty aldehyde may convert some of the fatty aldehyde to a fatty alcohol. When a microorganism that produces fatty alcohols is engineered to express a polynucleotide encoding an ester synthase, wax esters are produced. In a preferred embodiment, fatty alcohols are made from a fatty acid biosynthetic pathway. As an example, Acyl-ACP can be converted to fatty acids via the action of a thioesterase (e.g., E. coli tesA), which are converted to fatty aldehydes and fatty alcohols via the action of a carboxylic acid reductase (e.g., Mycobacterium carB, carA or fadD9). Conversion of fatty aldehydes to fatty alcohols can be further facilitated, for example, via the action of an alcohol dehydrogenase (e.g., E. coli YqhD, or Acinetobacter alrAadp1).

As used herein, the term “fatty acid” means a carboxylic acid having the formula RCOOH. R represents an aliphatic group, preferably an alkyl group. R can comprise between about 4 and about 22 carbon atoms. Fatty acids can be saturated or monounsaturated. In a preferred embodiment, the fatty acid is made from a fatty acid biosynthetic pathway.

As used herein, the term “fatty acid biosynthetic pathway” means a biosynthetic pathway that produces acyl thioesters. The fatty acid biosynthetic pathway includes fatty acid synthases that can be engineered to produce acyl thioesters, and in some embodiments can be expressed with additional enzymes to produce fatty acids having desired carbon chain characteristics. It is understood by those skilled in the art that fatty acids are biosynthesized not as the “acids”, but as acyl thioesters, i.e., the acid is bound as a thioester to the 4-phosphopantethionyl prosthetic group of ACP or CoA. The fatty acyl group can them be used in the cell to build membranes, cell walls, fats, hydrolyzed to fatty acids, and may be further modified biochemically to produce fatty acid derivatives, such as aldehydes, alcohols, alkenes, alkanes, esters, and the like.

As used herein, the term “fatty acid derivatives” means products made in part by way of the fatty acid biosynthetic pathway. The term “fatty acid derivatives” may be used interchangeably herein with the term “fatty acids or derivatives thereof” and includes products made in part from acyl-ACP or acyl-ACP derivatives. Exemplary “fatty acid derivatives” include, for example, fatty acids, acyl-CoA, fatty aldehydes, short and long chain alcohols, hydrocarbons (e.g., alkanes, alkenes or olefins, such as terminal or internal olefins), fatty alcohols, esters (e.g., wax esters, fatty acid esters (e.g., methyl or ethyl esters)), and ketones.

As used herein, the term “alkane” means saturated hydrocarbons or compounds that consist only of carbon (C) and hydrogen (H), wherein these atoms are linked together by single bonds (i.e., they are saturated compounds).

As used herein, the terms “olefin” and “alkene” are used interchangeably and refer to hydrocarbons containing at least one carbon-to-carbon double bond (i.e., they are unsaturated compounds).

As used herein, the terms “terminal olefin,” “α-olefin”, “terminal alkene” and “1-alkene” are used interchangeably herein with reference to α-olefins or alkenes with a chemical formula C_{X}H₂ₓ, distinguished from other olefins with a similar molecular formula by linearity of the hydrocarbon chain and the position of the double bond at the primary or alpha position.

As used herein, the term “fatty ester” refers to any ester made from a fatty acid, for example a fatty acid ester. In some embodiments, a fatty ester contains an A side and a B side. As used herein, an “A side” of an ester refers to the carbon chain attached to the carboxylate oxygen of the ester. As used herein, a “B side” of an ester refers to the carbon chain comprising the parent carboxylate of the ester. In embodiments where the fatty ester is derived from the fatty acid biosynthetic pathway, the A side is contributed by an alcohol (e.g., ethanol or methanol), and the B side is contributed by a fatty acid.

Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. When the fatty ester is a fatty acid methyl ester, the A side of the ester is 1 carbon in length. When the fatty ester is a fatty acid ethyl ester, the A side of the ester is 2 carbons in length. The B side of the ester can be at least about 4, 6, 8, 10, 12, 14, 16, 18, 20, 22, 24, or 26 carbons in length. Furthermore, the A side and/or B side can be saturated or unsaturated.

In one embodiment, the fatty ester is a wax. The wax can be derived from a long chain alcohol and a long chain fatty acid. In another embodiment, the fatty ester is a fatty acid thioester, for example Acyl-ACP. Fatty esters can be used, for example, as biofuels or surfactants.

As used herein, the term “recombinant host cell” refers to a host whose genetic makeup has been altered relative to the corresponding wild-type host cell, for example, by deliberate introduction of new genetic elements and/or deliberate modification of genetic elements naturally present in the host cell. The offspring of such recombinant host cells also contain these new and/or modified genetic elements. In any of the aspects of the invention described herein, the host cell can be selected from the group consisting of a mammalian cell, plant cell, insect cell, fungus cell (e.g., a filamentous fungus, such as Candida sp., or a budding yeast, such as Saccharomyces sp.), algal cell, and bacterial cell. In a preferred embodiment, recombinant host cells are “recombinant microorganisms.”

As used herein, a “host cell of the same kind as the recombinant host cell” typically means a host cell of the same species that does not have the recombinant modification described for the recombinant host cell. For example, “a microorganism of the same kind as the recombinant microorganism” typically refers to a microorganism of the same species, (e.g., E. coli), and the same strain (e.g., E. coli K-12) as the recombinant microorganism, wherein the microorganism does not comprise the recombinant modification described for the recombinant microorganism.

Examples of host cells that are microorganisms include but are not limited to the following. In some embodiments, the host cell is a Gram-positive bacterial cell. In other embodiments, the host cell is a Gram-negative bacterial cell.

In some embodiments, the host cell is selected from the genus Escherichia, Lactobacillus, Zymomonas, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In certain preferred embodiments, the host cell is an E. coli cell. In some embodiments, the E. coli cell is a strain B, a strain C, a strain K, or a strain W E. coli cell.

In other embodiments, the host cell is a Bacillus lentus cell, a Bacillus brevis cell, a Bacillus stearothermophilus cell, a Bacillus lichenoformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus ozyzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell.

In yet other embodiments, the host cell is an Actinomycetes cell.

In some embodiments, the host cell is a Saccharomyces cerevisiae cell. In some embodiments, the host cell is a Saccharomyces cerevisiae cell.

In other embodiments, the host cell is a cell from a eukaryotic plant, algae, cyanobacterium, green-sulfur bacterium, green non-sulfur bacterium, purple sulfur bacterium, purple non-sulfur bacterium, extremophile, yeast, fungus, algae, an engineered organism thereof, or a synthetic organism. In some embodiments, the host cell is light-dependent or fixes carbon. In some embodiments, the host cell is light-dependent or fixes carbon. In some embodiments, the host cell has autotrophic activity. In some embodiments, the host cell has photoautotrophic activity, such as in the presence of light. In some embodiments, the host cell is heterotrophic or mixotrophic in the absence of light. In certain embodiments, the host cell is a cell from Avabidopsis thaliana, Panicum virgatum, Miscanthus giganteus, Zea mays, Botryococcuse braunii, Chlamydomonas reinhardtii, Dunaliela salina, Synechococcus Sp. PCC 7002, Synechococcus Sp. PCC 7942, Synechocystis Sp. PCC 6803, Thermosynechococcus elongates BP-1, Chlorobium tepidum, Chlorojlexus auranticus, Chromatiumm vinosum, Rhodospirillum rubrum, Rhodobacter capsulatus, Rhodopseudomonas palusris, Clostridium ljungdahlii, Clostridiuthermocellum, Penicillium chrysogenum, Pichia pastoris, Saccharomyces cerevisiae, Schizosaccharomyces pombe, Pseudomonas jlorescens, or Zymomonas mobilis.

Examples of other host cells include, but are not limited to, a CHO cell, a COS cell, a VERO cell, a BHK cell, a HeLa cell, a Cv1 cell, an MDCK cell, a 293 cell, a 3T3 cell, or a PC12 cell.

As used herein, the term “clone” typically refers to a cell or group of cells descended from and essentially genetically identical to a single common ancestor, for example, the bacteria of a cloned bacterial colony arose from a single bacterial cell.

As used herein, the term “culture” typical refers to a liquid media comprising viable cells, in preferred embodiments the cells are obtained from a clone. In one embodiment a culture comprises cells reproducing in a predetermined culture media under controlled conditions, for example, a clone of a recombinant microorganism grown in liquid media comprising a selected carbon source and nitrogen.

As used herein, the term “fermentation” broadly refers to the conversion of organic materials into target substances by host cells, for example, the conversion of a carbon source by recombinant microorganisms into fatty acids or derivatives thereof by propagating a culture of the recombinant microorganisms in a media comprising the carbon source.

As used herein, “modified” activity of a protein, for example an enzyme, in a recombinant microorganism refers to a difference in one or more heritable characteristics in the activity determined relative to the parent microorganism. Typically differences in activity are determined between a recombinant microorganism, having modified activity, and the corresponding wild-type microorganism (e.g., comparison of a culture of a cloned, recombinant E. coli relative to wild-type E. coli). Modified activities can be the result of, for example, modified amounts of protein expressed by a recombinant microorganism (e.g., as the result of increased or decreased number of copies of DNA sequences encoding the protein, increased or decreased number of mRNA transcripts encoding the protein, and/or increased or decreased amounts of protein translation of the protein from mRNA); changes in the structure of the protein (e.g., changes to the primary structure, such as, changes to the protein's coding sequence that result in changes in substrate specificity, changes in observed kinetic parameters); and changes in protein stability (e.g., increased or decreased degradation of the protein). In some embodiments, the polypeptide is a mutant or a variant of any of the polypeptides described herein.

The term “regulatory sequences” as used herein typically refers to an element, such as a sequence of bases in DNA, that ultimately controls the expression of the protein. Examples of regulatory sequences include, but are not limited to, DNA promoter sequences, transcription factor binding sequences, transcription termination sequences, modulators of transcription (such as enhancer elements), nucleotide sequences that affect RNA stability, and translational regulatory sequences (such as, ribosome binding sites, initiation codons, termination codons).

As used herein, the phrase “the expression of said nucleotide sequence is modified relative to the wild type nucleotide sequence,” means an increase or decrease in the level of expression and/or activity of an endogenous nucleotide sequence or the expression and/or activity of a heterologous or non-native polypeptide-encoding nucleotide sequence. In some embodiments, an exogenous regulatory element that controls the expression of an endogenous or heterologous polynucleotide encoding a polypeptide is an expression control sequence that is operably linked to the endogenous or heterologous polynucleotide by recombinant integration into the genome of the host cell. In some embodiments, the expression control sequence is integrated into a host cell chromosome by homologous recombination using methods known in the art. In some embodiments, the polypeptide coding sequence is a mutant or a variant of any of the polypeptide coding sequences described herein.

As used herein, the terms “oxoacyl ACP synthase” and “β-ketoacyl-ACP synthase protein” are used interchangeable to refer to an enzyme of long-chain fatty acid synthesis that adds a two-carbon unit from malonyl-ACP (acyl carrier protein) to another molecule of fatty acyl-ACP, giving a β-ketoacyl-ACP with the release of carbon dioxide, for example, EC 2.3.1.41 enzymes. B-ketoacyl-ACP synthase (KAS) type III catalyzes an initial condensation reaction; as used herein the phrase “initial condensation β-ketoacyl-ACP synthase” refers to these types of polypeptides. KAS type I and type II are responsible for catalyzing the elongation steps in fatty acid biosynthesis; as used herein the phrase “elongation β-ketoacyl-ACP synthase” refers to these types of polypeptides. Enzymes of this group include, but are not limited to, 3-oxoacyl-[acyl-carrier-protein] synthase I (EC 2.3.1.41) and 3-oxoacyl-[acyl-carrier-protein] synthase II (EC 2.3.1.179), and enzymes identified by the numerical classification of the International Union of Biochemistry and Molecular Biology's Enzyme Commission numbers EC 2.3.1.-; The designation EC 2.3.1.- includes EC 2.3.1.X, where X is an integer, EC 2.3.1.nX, where X is an integer (preliminary EC numbers include an ‘n’ as part of the fourth (serial) digit, for example, where X=n1), and enzymes having the classification EC 2.3.1. Examples of proteins encoded by genes encoding such enzymes include, but are not limited to, fabB protein, E. coli (J Biol. Chem. 13; 279(33):34489-95 (2004)); fabF protein, E. coli (J Bacteriol. 169(4):1469-73 (1987)); CEM1 protein, S. cerevisiae, (Mol. Microbiol. 9(3):545-55 (1993)); KAS2 protein, Arabidopsis (Plant J 29(6):761-70 (2002)); and fabF protein, Enterococcus faecalis (J Biol. Chem. 13; 279(33):34489-95 (2004)). In preferred embodiments of the present invention the β-ketoacyl-ACP synthase protein is 3-oxoacyl-[acyl-carrier-protein] synthase I (EC 2.3.1.41) or 3-oxoacyl-[acyl-carrier-protein] synthase II (EC 2.3.1.179). Further examples of β-ketoacyl-ACP synthase protein are listed in Table 1 below.

As used herein, the term “acyl-ACP hydrolase” protein refers to enzymes of long-chain fatty acid synthesis that terminate fatty acyl group extension via hydrolyzing an acyl group on a fatty acid, typically those enzymes acting on thioester bonds that hydrolyzes the I-acyl bond. Enzymes of this group include, but are not limited to, acyl-ACP thioesterases, and enzymes identified by the numerical classification of the International Union of Biochemistry and Molecular Biology's Enzyme Commission numbers EC 3.1.1.5 or EC 3.1.2.-; The designation EC 3.1.2.- includes EC 3.1.2.X, where X is an integer, EC 3.1.2.nX, where X is an integer (preliminary EC numbers include an ‘n’ as part of the fourth (serial) digit, for example, where X=n1), and enzymes having the classification EC 3.1.2. Examples of proteins encoded by genes encoding such enzymes include, but are not limited to, tesA protein, E. coli (J Biol. Chem. 268: 9238-45 (1993)); fatB protein, Populus tomentosa (J. Genet. Genomics 34:267-273 (2007)); and Acyl-ACP thioesterase, Bacteroides thetaiotaomicron (Science 299:2074-2076 (2003)). Further examples of thioesterases are listed in Table 1 below.

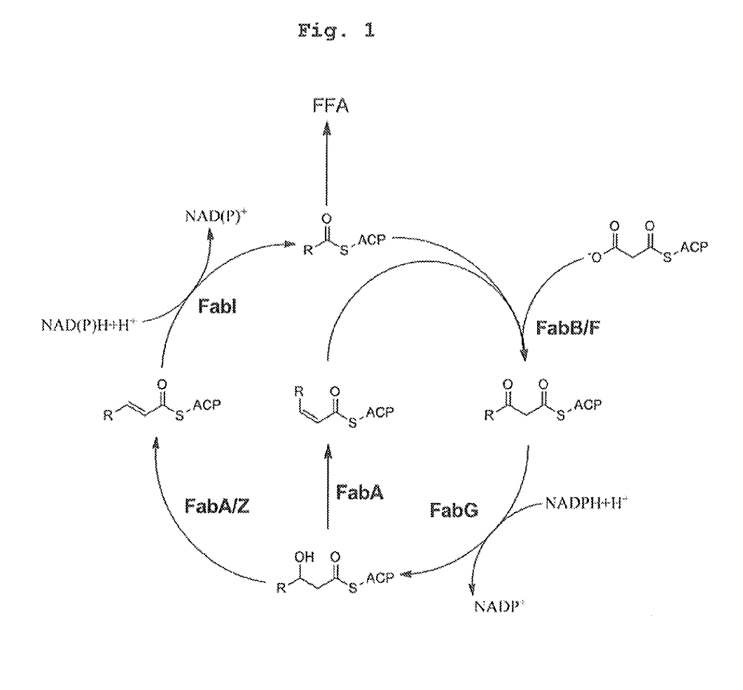

As used herein, the term “β-hydroxyacyl-ACP dehydratase” generally refers to enzymes of long-chain fatty acid synthesis that catalyze the dehydration of β-hydroxyacyl acyl carrier protein (ACP). Enzymes of this group include, but are not limited to, International Union of Biochemistry and Molecular Biology's Enzyme Commission numbers EC 4.2.1.- or EC 4.2.1.60; The designation EC 4.2.1.- includes EC 4.2.1.X, where X is an integer, EC 4.2.1.nX, where X is an integer (preliminary EC numbers include an ‘n’ as part of the fourth (serial) digit, for example, where X=n1), and enzymes having the classification EC 4.2.1. Examples of proteins encoded by genes encoding such enzymes include, but are not limited to, fabA protein, E. coli (Heath, R. J., et al., J Biol. Chem. 271(44):27795-801 (1996)); and fabZ protein, E. coli (Heath, R. J., et al., J Biol. Chem. 271(44):27795-801 (1996)). Further examples of β-hydroxyacyl-ACP dehydratase protein are listed in Table 1 below. E. coli fabA and fabZ encoded proteins catalyze the dehydration of β-hydroxyacyl ACP, as shown in FIG. 1. Subtle differences in substrate specificities for fabA and fabZ have been reported. For example, fabA has been reported to function as an isomerase, whereas fabZ has not. As used here, the term “titer” refers to the quantity of fatty acid or fatty acid derivative produced per unit volume of host cell culture. In any aspect of the compositions and methods described herein, a fatty acid or derivative thereof is produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 mg/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L (2 g/L), 3 g/L, 5g/L, 10 g/L, 20 g/L, 30 g/L, 40 g/L, 50 g/L, 60 g/L, 70 g/L, 80 g/L, 90 g/L, 100 g/L, 125 g/L, 150 g/L, 200 g/L, 250 g/L or a range bounded by any two of the foregoing values. In other embodiments, a fatty acid or fatty acid derivative is produced at a titer of more than 100 g/L, more than 200 g/L, more than 300 g/L, or higher, such as 500 g/L, 700 g/L, 1000 g/L, 1200 g/L, 1500 g/L, or 2000 g/L. According to some embodiments of the present invention, the preferred titer of a fatty acid or derivative thereof produced by a recombinant host cell is from 5 g/L to 200 g/L, 10 g/L to 150 g/L, 20 g/L to 120 g/L, 30 g/L to 100 g/L, or 30 g/L to 250 g/L.

As used herein, the term “yield of the fatty acid or derivative thereof produced by a host cell” refers to the efficiency by which an input carbon source is converted to product (i.e., fatty acid or fatty acid derivative such as fatty alcohol or fatty ester) by a host cell. Host cells engineered to produce fatty acids and fatty acid derivatives according to embodiments of the methods of the invention can have a yield of at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%%, at least 31%, at least 32%, at least 33%, at least 34%, at least 35%, at least 36%, at least 37%, at least 38%, at least 39%, or at least 40%, or a range bounded by any two of the foregoing values. In other embodiments, a fatty acid or fatty acid derivative is produced at a yield of more than 30%, 40%, 50%, 60%, 70%, 80%, 90% or more. Alternatively, or in addition, in some embodiments the yield is about 40% or less, about 37% or less, about 35% or less, about 32% or less, about 30% or less, about 27% or less, about 25% or less, or about 22% or less. Thus, the yield can be bounded by any two of the above endpoints. For example, the yield of the fatty acid or derivative thereof produced by embodiments of the recombinant host cell according to the methods of the invention can be 5% to 15%, 10% to 25%, 10% to 22%, 15% to 27%, 18% to 22%, 20% to 2S%, 20% to 30%, 15% to 30%, 10% to 30% or 10% to 40%. In preferred embodiments of the present invention, the yield of the fatty acid or derivative thereof produced by the recombinant host cell according to methods of the invention is from 10% to 30% or from 10% to 40%.

As used herein, the term “productivity of the fatty acid or derivative thereof produced” refers to the quantity of fatty acid or fatty acid derivative produced per unit volume of host cell culture per unit time. In any aspect of the compositions and methods described herein, the productivity of a fatty acid or a fatty acid derivative produced by a recombinant host cell is at least 100 mg/L/hour, at least 200 mg/L/hour, at least 300 mg/L/hour, at least 400 mg/L/hour, at least 500 mg/L/hour, at least 600 mg/L/hour, at least 700 mg/L/hour, at least 800 mg/L/hour, at least 900 mg/L/hour, at least 1000 mg/L/hour, at least 1100 mg/L/hour, at least 1200 mg/L/hour, at least 1300 mg/L/hour, at least 1400 mg/L/hour, at least 1500 mg/L/hour, at least 1600 mg/L/hour, at least 1700 mg/L/hour, at least 1800 mg/L/hour, at least 1900 mg/L/hour, at least 2000 mg/L/hour, at least 2100 mg/L/hour, at least 2200 mg/L/hour, at least 2300 mg/L/hour, at least 2400 mg/L/hour, at least 2500 mg/L/hour, at least 2600 mg/L/hour, at least 2700 mg/L/hour, at least 2800 mg/L/hour, at least 2900 mg/L/hour, or at least 3000 mg/L/hour. Alternatively, or in addition, in some embodiments the productivity is 3500 mg/L/hour or less, 3000 mg/L/hour or less, 2500 mg/L/hour or less, 2000 mg/L/hour or less, 1500 mg/L/hour or less, 120 mg/L/hour, or less, 1000 mg/L/hour or less, 800 mg/L/hour, or less, or 600 mg/I./hour or less. Thus, the productivity can be bounded by any two of the above endpoints. For example, in some embodiments the productivity can be 30 to 3000 mg/L/hour, 60 to 2000 mg/L/hour, or 100 to 1000 mg/L/hour. In preferred embodiments of the present invention, the productivity of a fatty acid or derivative thereof produced by a recombinant host cell according to methods of the invention is from 150 mg/L/hour to 1500 mg/L/hour, 500 mg/L/hour to 2500 mg/L/hour, or from 700 mg/L/hour to 3000 mg/L/hour.

As used herein, the term “over-express” means to express or cause to be expressed a polynucleotide or polypeptide in a cell at a greater concentration than is normally expressed in a corresponding wild-type cell under the same conditions. For example, a polynucleotide can be “over-expressed” in a recombinant host cell when.the polynucleotide is present in a greater concentration in the recombinant host cell as compared to its concentration in a non-recombinant host cell of the same species under the same conditions.

As used herein, the term “operably-linked” refers to a polynucleotide sequence and an expression control sequence(s) that are connected in such a way as to permit gene expression when the appropriate molecules (e.g., transcriptional activator proteins) are bound to the expression control sequence(s). Operably-linked promoters are located upstream of the selected polynucleotide sequence in terms of the direction of transcription and translation. Operably-linked enhancers can be located upstream, within, or downstream of the selected polynucleotide. Operably-linked translational control elements can be located outside of, within, or downstream of the protein coding sequences of a polynucleotide.

As used herein, the term “vector” refers to a nucleic acid molecule capable of transporting another nucleic acid, i.e., a polynucleotide sequence, to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids,” which refer generally to circular double stranded DNA loops that, in their vector form, are not bound to the chromosome. The terms “plasmid” and “vector” are used interchangeably herein, inasmuch as a plasmid is the most commonly used form of vector. However, also included are such other forms of expression vectors that serve equivalent functions and that become known in the art subsequently hereto.

Vectors can be introduced into prokaryotic or eukaryotic cells via conventional transformation or transfection techniques. As used herein, the terms “transformation” and “transfection” are used interchangeably to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Molecular Cloning: A Laboratory Manual (Third Edition), Sambrook, et al., Cold Spring Harbor Laboratory Press (2001).

As used herein, the term “under conditions effective to express said heterologous nucleotide sequences” means any conditions, that allow a host cell to produce a desired fatty acid or fatty acid derivative. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source that can be metabolized by a host cell directly. Fermentation denotes the use of a carbon source by a production host, such as a recombinant microorganism. Fermentation can be aerobic, anaerobic, or variations thereof (such as micro-aerobic). As will be appreciated by those of skill in the art, the conditions under which a recombinant microorganism can process a carbon source into acyl-ACP or a desired fatty acid or derivative thereof (e.g., a fatty ester, alkane, olefin, or an alcohol) will vary in part, based upon the specific microorganism. In some embodiments, the process occurs in an aerobic environment. In some embodiments, the process occurs in an anaerobic environment. In some embodiments, the process occurs in a micro-aerobic environment.

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; saturated or unsaturated fatty acids, succinate, lactate, and acetate; alcohols, such as ethanol, methanol, and glycerol, or mixtures thereof. The carbon source can also be a product of photosynthesis, such as glucose. In certain preferred embodiments, the carbon source is biomass. In other preferred embodiments, the carbon source is glucose, sucrose, fructose or combinations thereof. In other preferred embodiments, the carbon source is directly or indirectly derived from a natural feed stock such as sugar cane, sweet sorghum, switchgrass, sugar beets and others.

As used herein, the term “biomass” refers to any biological material from which a carbon source is derived. In some embodiments, a biomass is processed into a carbon source, which is suitable for bioconversion. In other embodiments, the biomass does not require further processing into a carbon source. The carbon source can be converted into any combination of fatty acids or fatty acid derivatives. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers. The term “biomass” also can refer to sources of carbon, such as carbohydrates (e.g., monosaccharides, disaccharides, or polysaccharides).

As used herein, the term “isolated,” with respect to products (such as fatty acids and derivatives thereof) refers to products that are separated from cellular components, cell culture media, or chemical or synthetic precursors. The fatty acids and derivatives thereof produced by the methods described herein can be relatively immiscible in the fermentation broth, as well as in the cytoplasm. Therefore, the fatty acids and derivatives thereof can collect in an organic phase either intracellularly or extracellularly. The collection of the products in the organic phase can lessen the impact of the fatty acid derivative, fatty aldehyde or fatty alcohol on cellular function and can allow the recombinant microorganism to produce more products. The fatty acids and derivatives thereof produced by the methods of invention generally are isolated from a liquid media in which the recombinant microorganisms are cultured.

As used herein, the terms “purify,” “purified,” or “purification” mean the removal or isolation of a molecule from its environment by, for example, isolation or separation. “Substantially purified” molecules are at least about 60% free (e.g., at least about 70% free, at least about 75% free, at least about 85% free, at least about 90% free, at least about 95% free, at least about 97% free, at least about 99% free) from other components with which they are associated. As used herein, these terms also refer to the removal of contaminants from a sample. For example, the removal of contaminants can result in an increase in the percentage of a fatty aldehyde or a fatty alcohol in a sample. For example, when a fatty aldehyde or a fatty alcohol is produced in a recombinant microorganism, the fatty aldehyde or fatty alcohol can be purified by the removal of recombinant microorganism proteins. After purification, the percentage of a fatty aldehyde or a fatty alcohol in the sample is increased. The terms “purify,” “purified,” and “purification” are relative terms that do not require absolute purity. Thus, for example, when a fatty aldehyde or a fatty alcohol is produced in recombinant microorganisms, a purified fatty aldehyde or a purified fatty alcohol is a fatty aldehyde or a fatty alcohol that is substantially separated from other cellular components (e.g., nucleic acids, polypeptides, lipids, carbohydrates, or other hydrocarbons).

As used herein, “fraction of modem carbon” or f_{M} has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

### General Overview of the Invention

Before describing the present invention in detail, it is to be understood that this invention is not limited to particular types of recombinant host cells, particular polynucleotide sequences, particular mutations, particular proteins, and the like, as use of such particulars may be selected in view of the teachings of the present specification. It is also to be understood that the terminology used herein is for the purpose of describing particular embodiments of the invention only, and is not intended to be limiting.

### Recombinant Host Cells and Recombinant Host Cell Cultures

In a first aspect, the present invention relates to recombinant host cell cultures engineered to produce high titer of a composition of fatty acid derivatives having target aliphatic chain lengths, the titer typically being between about 30 g/L to about 250 g/L. A large number of fatty acid derivatives can be produced by the recombinant host cells of the present invention, including, but not limited to, fatty acids, acyl-CoA, fatty aldehydes, short and long chain alcohols, hydrocarbons (e.g., alkanes, alkenes or olefins, such as terminal or internal olefins), fatty alcohols, esters (e.g., wax esters, fatty acid esters (e.g., methyl or ethyl esters), and ketones. In one embodiment, the present invention relates to the production of fatty alcohols.

In some embodiments of the present invention, the high titer of fatty acid derivatives produced by the recombinant host cells is a higher titer of fatty acid derivatives having selected aliphatic chain lengths relative to the titer of the same fatty acid derivatives produced by a control culture of wild-type host cells. Examples of such higher titers include, but are not limited to, the following: the recombinant host cell culture produces a higher titer of fatty alcohols having aliphatic chain lengths of C₈ relative to the titer of fatty alcohols having aliphatic chain lengths of C₈ produced by a control culture of a corresponding wild-type host cells; the recombinant host cell culture produces a higher titer of fatty alcohols having aliphatic chain lengths of C₈ and C₁₀ relative to the titer of fatty alcohols having aliphatic chain lengths of C₈ and C₁₀ produced by a control culture of a corresponding wild-type host cell; the recombinant host cell culture produces a higher titer of fatty alcohols having aliphatic chain lengths of C₁₂ relative to the titer of fatty alcohols having aliphatic chain lengths of C₁₂ produced by a control culture of a corresponding wild-type host cells; the recombinant host cell culture produces a higher titer of fatty alcohols having aliphatic chain lengths of C₁₂ and C₁₄ relative to the titer of fatty alcohols having aliphatic chain lengths of C₁₂ and C₁₄ produced by a control culture of a corresponding wild-type host cell; and, the recombinant host cell culture produces a higher titer of fatty alcohols having aliphatic chain lengths of C₁₂, C₁₄, and C₁₈, relative to the titer of fatty alcohols having aliphatic chain lengths of C₁₂, C₁₄, and C₁₈ produced by a control culture of a corresponding wild-type host cells. In other embodiments of the present invention, the higher titer of fatty acid derivatives is a higher titer of a particular type of fatty acid derivative (e.g., fatty alcohols, fatty acid esters, or hydrocarbons) relative to the titer of the same fatty acid derivative produced by a control culture of a corresponding wild-type host cell.

In a preferred embodiment of the present invention, the polynucleotide sequences comprise an open reading frame encoding an elongation β-ketoacyl-ACP synthase protein having an Enzyme Commission number of EC 2.3.1.- and operably-linked regulatory sequences that facilitate expression of the protein in recombinant host cells. In the recombinant host cells, the open reading frame coding sequences and/or the regulatory sequences are modified relative to the corresponding wild-type gene encoding the elongation β-ketoacyl-ACP synthase protein. The activity of the β-ketoacyl-ACP synthase protein in the recombinant host cell is modified relative to the activity of the β-ketoacyl-ACP synthase protein expressed from the wild-type gene in a corresponding host cell. Additionally, the recombinant host cells in the culture comprise one or more polynucleotide sequences that comprise an open reading frame encoding a thioesterase, having an Enzyme Commission number of EC 3.1.1.5 or EC 3.1.2.- and operably-linked regulatory sequences that facilitate expression of the protein in recombinant host cells. In the recombinant host cells, the open reading frame coding sequences and/or the regulatory sequences are modified relative to the corresponding wild-type gene encoding the thioesterase. The activity of the thioesterase in the recombinant host cell is modified relative to the activity of the thioesterase expressed from the corresponding wild-type gene in a corresponding host cell.

Methods of making proteins having modified enzymatic activities are described below. Further, exemplary recombinant host cells expressing proteins having such modified activities are described in the Examples.

One embodiment of the present invention is directed to a recombinant host cell culture that produces a high titer of a composition of fatty acid derivatives having a target aliphatic chain length. The recombinant host cell culture comprises recombinant host cells. The recombinant host cells are engineered to produce the composition of fatty acid derivatives having the target aliphatic chain length. The recombinant host cells typically comprise a modified activity of an elongation β-ketoacyl-ACP synthase protein, having an Enzyme Commission number of EC 2.3.1.-. The modified activity differs from the activity of the β-ketoacyl-ACP synthase protein produced by expression of a starting polynucleotide sequence (SPS_{A}) comprising an open reading frame polynucleotide sequence (ORF_{A}) encoding the elongation β-ketoacyl-ACP synthase protein, the ORF_{A} having 5′ and 3′ ends, and a 5′ non-coding polynucleotide sequence (NC_{A}) comprising operably-linked regulatory sequences adjacent the 5′-end of the ORF_{A}, in a host cell of the same kind as the recombinant host cell (e.g., a wild-type host cell from which the recombinant host cell was derived). The starting polynucleotide sequence can, for example, be a wild-type gene encoding the elongation β-ketoacyl-ACP synthase protein. Further, the recombinant host cells comprise one or more polynucleotide sequences, encoding the β-ketoacyl-ACP synthase protein and operably-linked regulatory sequences, comprising a variant ORF_{A} and/or a variant NC_{A} having less than 100% sequence identity to the ORF_{A} or the NC_{A}, respectively. In addition, the recombinant host cells comprise a modified activity of a thioesterase having an Enzyme Commission number of EC 3.1.1.5 or EC 3.1.2.-. The modified activity differs from the activity of the thioesterase produced by expression of a starting polynucleotide sequence (SPS_{B}) comprising an open reading frame polynucleotide sequence (ORF_{B}) encoding the thioesterase, the ORF having 5′ and 3′ ends, and a 5′ non-coding polynucleotide sequence (NC_{B}) comprising operably-linked regulatory sequences adjacent the 5′-end of the ORF_{B}, in a host cell of the same kind as the recombinant host cell. The starting polynucleotide sequence can, for example, be a wild-type gene encoding the thioesterase. Further, the recombinant host cells comprise one or more polynucleotide sequences, encoding the thioesterase and operably-linked regulatory sequences, comprising a variant ORF_{B} and/or a variant NC_{B} having less than 100% sequence identity to the ORF_{B} or the NC_{B}.

The recombinant host cell culture typically produces a fatty acid derivative composition with a high titer (between about 30 g/L and about 250 g/L) and having a target aliphatic chain length.

A recombinant culture typically produces a titer of fatty acid derivatives at least about 3 times greater, at least about 5 times greater, at least about 8 times greater, or at least about 10 times greater than the titer of fatty acid derivatives produced by a control culture propagated under the same conditions as the recombinant culture. Recombinant cultures typically comprise recombinant host cells comprising mutagenized polynucleotide sequences (having an open reading frame encoding a protein operably-linked to regulatory sequences that facilitate expression of the protein). Control cultures typically comprise host cells expressing the wild-type genes encoding the elongation β-ketoacyl-ACP synthase protein and the thioesterase. Alternatively, control cultures can comprise host cells comprising polynucleotide sequences (having an open reading frame encoding a protein operably-linked to regulatory sequences that facilitate expression of the protein) that were used as the starting polynucleotide sequences for mutagenesis before introduction into the recombinant host cells of the present invention. In some embodiments, the recombinant host cell culture produces a titer of fatty acid derivatives of from about 30 g/L to about 250 g/L.

In some embodiments of the present invention, the recombinant host cell culture produces a yield of fatty acid derivatives of at least about 3 times greater, about 5 times greater, about 8 times greater, or about 10 times greater than the titer of fatty acid derivatives produced by a control culture propagated under the same conditions as the recombinant culture. Examples of fatty acid derivative yields include production by the recombinant host cell culture of fatty acid derivatives of between about 10% to about 40%. Typically, titer and yield have a positive correlation.

In some embodiments, the recombinant host cell culture's productivity of fatty acid derivatives is at least about 3 times greater, about 5 times greater, about 8 times greater, or about 10 times greater than a control culture's productivity when propagated under the same conditions as the recombinant culture. Examples of fatty acid derivative productivity by the recombinant host cell culture include between about 700 mg/L/hour to about 3000 mg/L/hour. Typically, titer and productivity have a positive correlation.

In one embodiment of the present invention, the recombinant host cell culture is propagated in a media comprising a carbon source. Suitable carbon sources include, but are not limited to, monosaccharides (e.g., glucose), disaccharides (e.g., sucrose), oligosaccharides, polysaccharides (e.g., cellulose or starch), cellulosic materials, and biomass.

In the recombinant host cell culture of any of the preceding embodiments, examples of the nucleotide sequence encoding the β-ketoacyl-ACP synthase protein include, but are not limited to, sequences encoding 3-oxoacyl-[acyl-carrier-protein] synthase I protein (Enzyme Commission number EC 2.3.1.41) or 3-oxoacyl-[acyl-carrier-protein] synthase II protein (Enzyme Commission number EC 2.3.1.179). In a preferred embodiment using 3-oxoacyl-[acyl-carrier-protein] synthase I protein, the synthase protein ORF_{A} encodes an E. coli fabB derived 3-oxoacyl-[acyl-carrier-protein] synthase I protein that has the sequence set forth in SEQ ID NO:2, and the variant synthase protein ORF_{A} encodes a 3-oxoacyl-[acyl-carrier-protein] synthase I protein that has at least about 70%, about 75%, about 80%, about 85%, preferably about 90% or about 95% or greater sequence identity to the E. coli fabB protein (SEQ ID NO:2). In a preferred embodiment using 3-oxoacyl-[acyl-carrier-protein] synthase II protein, the synthase protein ORF_{A} encodes an E. coli fabF derived 3-oxoacyl-[acyl-carrier-protein] synthase II protein that has the sequence set forth in SEQ ID NO:4, and the variant synthase protein ORF_{A} encodes a 3-oxoacyl-[acyl-carrier-protein] synthase II protein that has at least about 70%, about 75%, about 80%, about 85%, preferably about 90% or about 95% or greater sequence identity to the E. coli fabF protein (SEQ ID NO:4). Further, a variant 5′ non-coding polynucleotide sequence, variant NC_{A}, can be provided, for example, from a library generated by randomization of the NC_{A}. Variant non-coding polynucleotide sequences (e.g., variant NC_{A}) typically have from zero percent sequence identity to <100% percent sequence identity when compared to the starting non-coding polynucleotide sequences (e.g., NC_{A}).

In the recombinant host cell culture of any of the preceding embodiments, examples of the nucleotide sequence encoding the thioesterase include, but are not limited to, sequences encoding a thioesterase protein (Enzyme Commission numbers of EC 3.1.1.5 or EC 3.1.2.-). In preferred embodiments using the thioesterase protein, the thioesterase protein ORF_{B} encodes an E. coli tesA derived thioesterase protein that has the sequence set forth in SEQ ID NO:6, SEQ ID NO:8, SEQ ID NO:17, or SEQ ID NO:19, and the variant ORF_{B} encodes a thioesterase protein that has at least about 70%, about 75%, about 80%, about 85%, preferably about 90% or about 95% or greater sequence identity to the E. coli tesA protein (SEQ ID NO:6, SEQ ID NO:8, SEQ ID NO:17, or SEQ ID NO:19, respectively). Further, a variant 5′ non-coding polynucleotide sequence, variant NC_{B}, can be provided, for example, from a library generated by randomization of the NC_{B}. Variant non-coding polynucleotide sequences (e.g., variant NC_{B}) typically have from zero percent sequence identity to <100% percent sequence identity when compared to the starting non-coding polynucleotide sequences (e.g., NC_{B}).

The recombinant host cells of the cultures of the present invention can further comprise one or more nucleotide sequence encoding a carboxylic acid reductase protein that has an Enzyme Commission number of EC 6.2.1.3 or EC 1.2.1.42, and operably-linked regulatory sequences. In a preferred embodiment, the carboxylic acid reductase protein is a protein that has at least about 70%, about 75%, about 80%, about 85%, preferably about 90% or about 95% or greater sequence identity to a Mycobacterium smegmatis carB fatty acid reductase protein (SEQ ID NO:10). In other embodiments, the carboxylic acid reductase protein is a protein that has at least about 70%, about 75%, about 80%, about 85%, preferably about 90% or about 95% or greater sequence identity to (i) a Mycobacterium tuberculosis fadD9 protein (SEQ ID NO:21; see, also, US Patent Publication No. 20100105963), or (ii) a Mycobacterium smegmatis carA protein (SEQ ID NO:23; see, also, US Patent Publication No. 20100105963).

In addition, the recombinant host cells of the present invention can further comprise one or more polynucleotide sequences encoding an alcohol dehydrogenase protein having an Enzyme Commission number of EC 1.1.-.-, EC 1.1.1.1, or EC 1.2.1.10, and operably-linked regulatory sequences. Examples of such alcohol dehydrogenase proteins include, but are not limited to, E. coli AdhE, aldehyde-alcohol dehydrogenase protein, or E. coli yqhD, alcohol dehydrogenase protein.

In the recombinant host cell cultures of the present invention, the high titer of fatty acid derivatives can be, a high titer of the fatty acid derivative having aliphatic chain lengths selected from the group of aliphatic chains lengths consisting of between C₈, C₁₀, C₁₂, C₁₄, C₁₆, C₁₈, C₂₀, and combinations thereof. The high titer of fatty acid derivatives can be, for example, a high titer of fatty alcohols having aliphatic chain lengths of C₈, a high titer of fatty alcohols having aliphatic chain lengths of C₁₀, a high titer of fatty alcohols having aliphatic chain lengths of C₁₂, a high titer of fatty alcohols having aliphatic chain lengths of C₁₄, a high titer of fatty alcohols having aliphatic chain lengths of C₁₆, a high titer of fatty alcohols having aliphatic chain lengths of C₁₈, a high titer of fatty alcohols having aliphatic chain lengths of C₂₀, as well as combinations thereof. In one embodiment, a ratio (C_{X}/C_{Y}) of two selected aliphatic chain lengths is used to characterize the aliphatic chain length. The C_{X}/C_{Y} ratio is the titer of fatty acid derivatives having an aliphatic chain length of C_{X} to the titer of fatty acid derivatives having an aliphatic chain length of C_{Y}. In some embodiments of the present invention, C_{X}/C_{Y} has a value of between about 1.5 to about 6, where X and Y are integer values and X is less than Y. In other embodiments of the present invention, C_{X}/C_{Y} has a value of at least about 2, where X and Y are integer values and X is less than Y. In a preferred embodiment, C_{X}/C_{Y} has a value of between about 2 and about 4, where X and Y are integer values and X is less than Y. Examples of X and Y values include, but are not limited to: X=8, Y=10; X=12, Y=14; X=14, Y=16; and X=18, Y=20. Other combinations of X and Y values are readily apparent to one of ordinary skill in the art in view of the teachings of the present specification.

A second aspect of the present invention relates to providing a desired degree of saturation of the aliphatic chains of the fatty acid derivatives (e.g., fatty alcohols). In this aspect, the recombinant host cells as described above further comprise one or more polynucleotide sequences that comprise an open reading frame encoding a β-hydroxyacyl-ACP dehydratase protein, having an Enzyme Commission number of EC 4.2.1.- or 4.2.1.60, and operably-linked regulatory sequences that facilitate expression of the protein in recombinant host cells. In the recombinant host cells, the open reading frame coding, sequences and/or the regulatory sequences are modified relative to the corresponding wild-type gene encoding the β-hydroxyacyl-ACP dehydratase protein. The activity of the β-hydroxyacyl-ACP dehydratase protein in the recombinant host cell is modified relative to the activity of the β-hydroxyacyl-ACP dehydratase protein expressed from the wild-type gene in a corresponding host cell.

In some embodiments, the modified activity differs from the activity of the β-hydroxyacyl-ACP dehydratase protein produced by expression of a starting polynucleotide sequence (SPS_{C}) comprising an open reading frame polynucleotide sequence (ORF_{C}) encoding the β-hydroxyacyl-ACP dehydratase protein, the ORF_{C} having 5′ and 3′ ends, and a 5′ non-coding polynucleotide sequence (NC_{C}) comprising operably-linked regulatory sequences adjacent the 5′-end of the ORF_{C}, in a host cell of the same kind as the recombinant host cell. The recombinant host cell typically comprises one or more polynucleotide sequences, encoding the β-hydroxyacyl-ACP dehydratase protein and operably-linked regulatory sequences, comprising a variant ORF_{C} and/or a variant NC_{C} having less than 100% sequence identity to the ORF_{C} or the NC_{C}, respectively.

In some embodiments, the ORF_{C} encodes an E. coli fabZ derived (3R)-hydroxymyristol acyl carrier protein dehydratase protein that has the sequence set forth in SEQ ID NO:14, and the variant ORF_{C} encodes a (3R)-hydroxymyristol acyl carrier protein dehydratase protein that has at least about 70%, about 75%, about 80%, about 85%, preferably about 90% or about 95% or greater sequence identity to the E. coli fabZ protein (SEQ ID NO:14). In some embodiments, the ORF_{C} encodes an E. coli fabA derived β-hydroxydecanoyl thioester dehydratase/isomerase protein that has the sequence set forth in SEQ ID NO:12, and the variant ORF_{C} encodes a β-hydroxydecanoyl thioester dehydratase/isomerase protein that has at least about 70%, about 75%, about 80%, about 85%, preferably about 90% or about 95% or greater sequence identity to an E. coli fabA protein (SEQ ID NO:12).

Further, a variant 5′ non-coding polynucleotide sequence, variant NC_{C}, can be provided, for example, from a library generated by randomization of the NC_{C}. Variant non-coding polynucleotide sequences (e.g., variant NC_{C}) typically have from zero percent sequence identity to <100% percent sequence identity when compared to the starting non-coding polynucleotide sequences (e.g., NC_{C}).

In one embodiment, the composition of fatty acid derivatives having the target aliphatic chain length further has a preferred percent saturation. For example, the composition of fatty acid derivatives having the target aliphatic chain length comprise saturated and unsaturated aliphatic chains, and at least about 90% of the target fatty acid derivatives have saturated aliphatic chains. Following the teachings of the present specification one of ordinary skill in the art can select a desired percent saturation of the target fatty acid derivatives.

A third aspect of the present invention relates to recombinant host cell cultures that produce compositions of fatty acid derivatives having target aliphatic chain lengths. The recombinant host cell cultures comprise recombinant host cells. The recombinant host cells are engineered to produce the composition of fatty acid derivatives having the target aliphatic chain length. The recombinant host cells typically have a modified activity of a β-hydroxyacyl-ACP dehydratase protein, having an Enzyme Commission number of EC 4.2.1.- or 4.2.1.60. The modified activity differs from the activity of theβ-hydroxyacyl-ACP dehydratase protein produced by expression of a starting polynucleotide sequence (SPS_{D}) comprising an open reading frame polynucleotide sequence (ORF_{D}) encoding the β-hydroxyacyl-ACP dehydratase protein, the ORF_{D} having 5′ and 3′ ends, and a 5′ non-coding polynucleotide sequence (NC_{D}) comprising operably-linked regulatory sequences adjacent the 5′-end of the ORF_{D}, in a host cell of the same kind as the recombinant host cell. The recombinant host cells comprise one or more variants of the SPS_{D}, encoding the β-hydroxyacyl-ACP dehydratase protein and operably-linked regulatory sequences, comprising a variant ORF_{D} and/or a variant NC_{D} having less than 100% sequence identity to the ORF_{D} or the NC_{D}, respectively. The composition of fatty acid derivatives having the target aliphatic chain length produced by the recombinant host cell culture comprises a higher titer of fatty acid derivatives having the target aliphatic chain length than a fatty acid derivative composition produced by a culture of the host cell of the same kind as the recombinant host cell expressing the SPS_{D}. The starting polynucleotide sequence can be, for example, a wild-type gene encoding the β-hydroxyacyl-ACP dehydratase protein.

In some embodiments, the ORF_{D} encodes an E. coli fabZ derived (3R)-hydroxymyristol acyl carrier protein dehydratase protein that has the sequence set forth in SEQ ID NO:14, and the variant ORF_{D} encodes a (3R)-hydroxymyristol acyl carrier protein dehydratase protein that has at least about 70%, about 75%, about 80%, about 85%, preferably about 90% or about 95% or greater sequence identity to the E. coli fabZ protein (SEQ ID NO:14). In some embodiments, the ORF_{D} encodes an E. coli fabA derived β-hydroxydecanoyl thioester dehydratase/isomerase protein that has the sequence set forth in SEQ ID NO:12, and the variant ORF_{D} encodes a β-hydroxydecanoyl thioester dehydratase/isomerase protein that has at least about 70%, about 75%, about 80%, about 85%, preferably about 90% or about 95% or greater sequence identity to an E. coli fabA protein (SEQ ID NO:12).

Further, a variant 5′ non-coding polynucleotide sequence, variant NC_{D}, can be provided, for example, from a library generated by randomization of the NC_{D}. Variant non-coding polynucleotide sequences (e.g., variant NC_{D}) typically have from zero percent sequence identity to <100% percent sequence identity when compared to the starting non-coding polynucleotide sequences (e.g., NC_{D}).

Recombinant host cells of this third aspect of the present invention can further comprise additional elements as described herein, for example, elongation β-ketoacyl-ACP synthase genes, acyl-ACP hydrolase genes, carboxylic acid reductase genes, alcohol dehydrogenase genes, and so on.

In a fourth aspect the present invention relates to recombinant host cell cultures that produce compositions of fatty acid derivatives having preferred percent saturation. The recombinant host cell culture comprises recombinant host cells engineered to produce the compositions of fatty acid derivatives having the preferred percent saturation. The recombinant host cells comprise a modified activity of a β-hydroxyacyl-ACP dehydratase protein that lacks isomerase activity, having an Enzyme Commission number of EC 4.2.1.-. The modified activity differs from the activity of the β-hydroxyacyl-ACP dehydratase protein that lacks isomerase activity produced by expression of a starting polynucleotide sequence (SSP_{E}) comprising an open reading frame polynucleotide sequence (ORF_{E}) encoding the β-hydroxyacyl-ACP dehydratase protein that lacks isomerase activity, the ORF_{E} having 5′ and 3′ ends, and a 5′ non-coding polynucleotide sequence (NC_{E}) comprising operably-linked regulatory sequences adjacent the 5′-end of the ORF_{E}, in a host cell of the same kind as the recombinant host cell. The recombinant host cell comprises one or more polynucleotide sequences, encoding the β-hydroxyacyl-ACP dehydratase protein that lacks isomerase activity and operably-linked regulatory sequences, comprising a variant ORF_{E} and/or a variant NC_{E} having less than 100% sequence identity to the ORF_{E} or the NC_{E}, respectively. The composition of fatty acid derivatives having the preferred percent saturation produced by the recombinant host cell culture comprises a higher titer of fatty acid derivatives having the preferred percent saturation than a fatty acid derivative composition produced by a culture of the host cell, of the same kind as the recombinant host cell, expressing the SPS_{E}. The starting polynucleotide sequence can be, for example, a wild-type gene encoding the β-hydroxyacyl-ACP dehydratase protein that lacks isomerase activity.

In some embodiments, the ORF_{E} encodes an E. coli fabZ derived (3R)-hydroxymyristol acyl carrier protein dehydratase protein that has the sequence set forth in SEQ ID NO: 14, and the variant ORF_{E} encodes a (3R)-hydroxymyristol acyl carrier protein dehydratase protein that has at least about 70%, about 75%, about 80%, about 85%, preferably about 90% or about 95% or greater sequence identity to the E. coli fabZ protein (SEQ ID NO:14).

Further, a variant 5′ non-coding polynucleotide sequence, variant NC_{E}, can be provided, for example, from a library generated by randomization of the NC_{E}. Variant non-coding polynucleotide sequences (e.g., variant NC_{E}) typically have from zero percent sequence identity to <100% percent sequence identity when compared to the starting non-coding polynucleotide sequences (e.g., NC_{E}).

Recombinant host cells of this fourth aspect of the present invention can further comprise additional elements as described herein, for example, elongation β-ketoacyl-ACP synthase genes, acyl-ACP hydrolase genes, carboxylic acid reductase genes, alcohol dehydrogenase genes, and so on.

In the recombinant host cell cultures described herein, the recombinant host cell can be a mammalian cell, plant cell, insect cell, fungus cell, algal cell or a bacterial cell. In one embodiment, the recombinant host cell is a microorganism (e.g., bacteria or fungi). In preferred embodiments, the recombinant host cells are bacteria. In a preferred embodiment, the bacteria are Escherichia coli.

In some embodiments of the present invention, the “fatty acid derivative” is fatty alcohol.

In some embodiments of the recombinant host cells and cultures of the present invention, the operably-linked regulatory sequences can confer constitutive expression or regulatable expression of the operably-linked open reading frame; resulting in constitutive or regulatable expression of the protein encoded by the open reading frame. For example, the expression of a protein in a host cell can be mediated via a constitutive promoter, or via an inducible/repressible promoter. Examples of inducible/repressible promoters include, but are not limited to, the following: the E. coli lac operon promoter, wherein inducers of the lac operon, such as IPTG (isopropyl-beta-D-thiogalactopyranoside) or allolactose (the natural inducer), bind the lac repressor it is no longer able to act on the promoter and transcription of genes under the control of the promoter are de-repressed; and GAL4-inducible promoters.

The one or more polynucleotide sequences, comprising open reading frames encoding proteins and operably-linked regulatory sequences can be integrated into a chromosome of the recombinant host cells, incorporated in one or more plasmid expression systems resident in the recombinant host cells, or both. In the Examples, plasmid expression systems are typically used to illustrate embodiments of the present invention.

Embodiments of the recombinant host cells of the cultures of present invention can further comprise one or more polynucleotide sequence encoding one or more additional proteins and operably-linked regulatory sequences. Examples of such additional proteins include, but are not limited to, acetyl-CoA acetyltransferase; β-hydroxybutyryl-CoA dehydrogenase; crotonase butyryl-CoA dehydryogenase; and coenzyme A-acylating aldehyde dehydrogenase. Such additional proteins can be expressed in the recombinant host cells to facilitate production of particular fatty acid derivatives from acyl-ACPs as substrates (see, e.g., FIG. 2 and Table 1).

TABLE 1

| | | | | |
|---|---|---|---|---|
| Gene | Source | | Accession | EC |
| Designation | Organism | Enzyme Name | No. | Number |

1. Fatty Acid Production Increase/Product Production Increase

| | | | | |
|---|---|---|---|---|
| accA | E. coli, | Acetyl-CoA carboxylase, | AAC73296, | 6.4.1.2 |
| | Lactococci | subunit A (carboxyltransferase | NP_414727 | |
| | | alpha) | | |
| accB | E. coli, | Acetyl-CoA carboxylase, | NP_417721 | 6.4.1.2 |
| | Lactococci | subunit B (BCCP: biotin | | |
| | | carboxyl carrier protein) | | |
| accC | E. coli, | Acetyl-CoA carboxylase, | NP_417722 | 6.4.1.2, |
| | Lactococci | subunit C (biotin carboxylase) | | 6.3.4.14 |
| accD | E. coli, | Acetyl-CoA carboxylase, | NP_416819 | 6.4.1.2 |
| | Lactococci | subunit D (carboxyltransferase | | |
| | | beta) | | |
| fadD | E. coli W3110 | acyl-CoA synthase | AP_002424 | 2.3.1.86, |
| | | | | 6.2.1.3 |
| fabA | E. coli K12 | β-hydroxydecanoyl thioester | NP_415474 | 4.2.1.60 |
| | | dehydratase/isomerase | | |
| fabB | E. coli | 3-oxoacyl-[acyl-carrier-protein] | BAA16180 | 2.3.1.41 |
| | | synthase I | | |
| fabD | E. coli K12 | [acyl-carrier-protein]S- | AAC74176 | 2.3.1.39 |
| | | malonyltransfcrasc | | |
| fabF | E. coli K12 | 3-oxoacyl-[acyl-carrier-protein] | AAC74179 | 2.3.1.179 |
| | | synthase II | | |
| fabG | E. coli K12 | 3-oxoacyl-[acyl-carrier protein] | AAC74177 | 1.1.1.100 |
| | | reductase | | |
| fabH | E. coli K12 | 3-oxoacyl-[acyl-carrier-protein] | AAC74175 | 2.3.1.180 |
| | | synthase III | | |
| fabI | E. coli K12 | enoyl-[acyl-carrier-protein] | NP_415804 | 1.3.1.9 |
| | | reductase | | |
| fabR | E. coli K12 | Transcriptional Repressor | NP_418398 | none |
| fabV | Vibrio cholerae | enoyl-[acyl-carrier-protein] | YP_001217283 | 1.3.1.9 |
| | | reductase | | |
| fabZ | E. coli K12 | (3R)-hydroxymyristol acyl | NP_414722 | 4.2.1.- |
| | | carrier protein dehydratase | | |
| fadE | E. coli K13 | acyl-CoA dehydrogenase | AAC73325 | 1.3.99.3, |
| | | | | 1.3.99.- |
| fadR | E. coli | transcriptional regulatory | NP_415705 | none |
| | | protein | | |

2. Chain Length Control

| | | | | |
|---|---|---|---|---|
| tesA (with | E. coli | thioesterase—leader sequence | P0ADA1 | 3.1.2.-, |
| or without | | is amino acids 1-26 | | 3.1.1.5 |
| leader | | | | |
| sequence) | | | | |
| tesA | E. coli | thioesterase | AAC73596, | 3.1.2.-, |
| (without | | | NP_415027 | 3.1.1.5 |
| leader | | | | |
| sequence) | | | | |
| tesA | E. coli | thioesterase | L109P | 3.1.2.-, |
| (mutant of | | | | 3.1.1.5 |
| E. coli | | | | |
| thioesterase | | | | |
| 1 complexed | | | | |
| with | | | | |
| octanoic | | | | |
| acid) | | | | |
| fatB1 | Umbellularia | thioesterase | Q41635 | 3.1.2.14 |
| | californica | | | |
| fatB2 | Cuphea | thioesterase | AAC49269 | 3.1.2.14 |
| | hookeriana | | | |
| fatB3 | Cuphea | thioesterase | AAC72881 | 3.1.2.14 |
| | hookeriana | | | |
| fatB | Cinnamomum | thioesterase | Q39473 | 3.1.2.14 |
| | camphora | | | |
| fatB | Arabidopsis | thioesterase | CAA85388 | 3.1.2.14 |
| | thaliana | | | |
| fatA1 | Helianthus | thioesterase | AAL79361 | 3.1.2.14 |
| | annuus | | | |
| atfata | Arabidopsis | thioesterase | NP_189147, | 3.1.2.14 |
| | thaliana | | NP_193041 | |
| fatA | Brassica juncea | thioesterase | CAC39106 | 3.1.2.14 |
| fatA | Cuphea | thioesterase | AAC72883 | 3.1.2.14 |
| | hookeriana | | | |
| tes | Photbacerium | thioesterase | YP_130990 | 3.1.2.14 |
| | profundum | | | |
| tesB | E. coli | thioesterase | NP_414986 | 3.1.2.14 |
| fadM | E. coli | thioesterase | NP_414977 | 3.1.2.14 |
| yciA | E. coli | thioesterase | NP_415769 | 3.1.2.14 |
| ybgC | E. coli | thioesterase | NP_415264 | 3.1.2.14 |

3. Saturation Level Control*

| | | | | |
|---|---|---|---|---|
| Sfa | E. coli | Suppressor of fabA | AAN79592, | none |
| | | | AAC44390 | |
| fabA | E. coli K12 | β-hydroxydecanoyl thioester | NP_415474 | 4.2.1.60 |
| | | dchydratasc/isomcrasc | | |
| GnsA | E. coli | suppressors of the secG null | ABD18647.1 | none |
| | | mutation | | |
| GnsB | E. coli | suppressors of the secG null | AAC74076.1 | none |
| | | mutation | | |
| fabB | E. coli | 3-oxoacyl-[acyl-carrier-protein] | BAA16180 | 2.3.1.41 |
| | | synthase I | | |
| fabK | Streptococcus | trans-2-enoyl-ACP reductase II | AAF98273 | 1.3.1.9 |
| | pneumoniae | | | |
| fabL | Bacillus | enoyl-(acyl carrier protein) | AAU39821 | 1.3.1.9 |
| | licheniformis | reductase | | |
| | DSM 13 | | | |
| fabM | Streptococcus | trans-2, cis-3-decenoyl-ACP | DAA05501 | 4.2.1.17 |
| | mutans | isomerase | | |
| des | Bacillus subtilis | D5 fatty acyl desaturase | O34653 | 1.14.19 |

4. Product Output: wax production

| | | | | |
|---|---|---|---|---|
| AT3G51970 | Arabidopsis | long-chain-alcohol O-fatty- | NP_I90765 | 2.3.1.26 |
| | thaliana | acyltransferase | | |
| ELO1 | Pichia angusta | Fatty acid elongase | BAD98251 | 2.3.1.- |
| plsC | Saccharomyces | acyltransferase | AAA16514 | 2.3.1.51 |
| | cerevisiae | | | |
| DAGAT/D | Arahidopsis | diacylglycerol acyltransferase | AAF19262 | 2.3.1.20 |
| GAT | thaliana | | | |
| hWS | Homo sapiens | acyl-CoA wax alcohol | AAX48018 | 2.3.1.20 |
| | | acyltransferase | | |
| aft1 | Acinetobacter | bifunctional wax ester | AAO17391 | 2.3.1.20 |
| | sp. ADP1 | synthase/acyl- | | |
| | | CoA:diacylglycerol | | |
| | | acyltransferase | | |
| WS377 | Marinobacter | wax ester synthase | ABO21021 | 2.3.1.20 |
| | hydrocarbonocl | | | |
| | asticus | | | |
| mWS | Simmondsia | wax ester synthase | AAD38041 | 2.3.1.- |
| | chinensis | | | |

5. Product Output: Fatty Alcohol Output

| | | | | |
|---|---|---|---|---|
| | | thioesterases (see above) | | |
| BmFAR | Bombyx mori | FAR (fatty alcohol forming | BAC79425 | 1.1.1.- |
| | | acyl-CoA reductase) | | |
| acrl | Acinetobacter | acyl-CoA reductase | YP_047869 | 1.2.1.42 |
| | sp. ADP1 | | | |
| ycihD | E. coli W3110 | alcohol dehydrogenase | AP_003562 | 1.1.-.- |
| alrA | Acinetobacter | alcohol dehydrogenase | CAG70252 | 1.1.-.- |
| | sp. ADP1 | | | |
| BmFAR | Bombyx mori | FAR (fatty alcohol forming | BAC79425 | 1.1.1.- |
| | | acyl-CoA reductase) | | |
| GTNG_1865 | Geobacillus | Long-chain aldehyde | YP_001125970 | 1.2.1.3 |
| | thermodenitrific | dehydrogenase | | |
| | ans NG80-2 | | | |
| AAR | Synechococcus | Acyl-ACP reductase | YP_400611 | 1.2..42 |
| | elongatus | | | |
| carB | Mycobacterium | carboxylic acid reductase | YP_889972 | 6.2.1.3, |
| | smegmatis | protein | | 1.2. I.42 |
| carA | Mycobacterium | carboxylic acid reductase | ABK75684 | 6.2.1.3, |
| | smegmatis | protein | | 1.2..42 |
| fadD9 | Mycobacterium | carboxylic acid reductase | NP_217106 | 6.2.1.3, |
| | tuberculosis | protein | | 1.2..42 |
| FadD | E. coli K12 | acyl-CoA synthetase | NP_416319 | 6.2.1.3 |
| atoB | Erwinia | acetyl-CoA acetyltransferase | YP_049388 | 2.3.19 |
| | carotovora | | | |
| hbd | Butyrivibrio | Beta-hydroxybutyryl-CoA | BAD51424 | 1.1.1.157 |
| | fibrisolvens | dehydrogenase | | |
| CPE0095 | Clostridium | crotonase butyryl-CoA | BAB79801 | 4.2.1.55 |
| | perfringens | dehydryogenase | | |
| bcd | Clostridium | butyryl-CoA dehydryogenase | AAM14583 | 1.3.99.2 |
| | beijerinckii | | | |
| ALDH | Clostridium | coenzyme A-acylating aldehyde | AAT66436 | 1.2.1.3 |
| | beijerinckii | dehydrogenase | | |
| AdhE | E. coli CET073 | aldehyde-alcohol | AAN80172 | 1.1.1.1 |
| | | dehydrogenase | | 1.2.1.10 |

6. Fatty Alcohol Acetyl Ester Output

| | | | | |
|---|---|---|---|---|
| | | thioesterases (see above) | | |
| acrl | Acinetobacter | acyl-CoA reductase | YP_047869 | 1.2.1.42 |
| | sp. ADP1 | | | |
| yqhD | E. Coli K12 | alcohol dehydrogenase | AP_003562 | 1.1.-.- |
| AAT | Fragaria x | alcohol O-acetyltransferase | AAG13130 | 2.3.1.84 |
| | ananassa | | | |

7. Product Export

| | | | | |
|---|---|---|---|---|
| AtMRP5 | Arabidopsis | Arabidopsis thaliana multidrug | NP_171908 | none |
| | thaliana | resistance-associated | | |
| AmiS2 | Rhodococcus | ABC transporter AmiS2 | JC5491 | none |
| | sp. | | | |
| AtPGP1 | Arabidopsis | Arabidopsis thaliana p | NP_181228 | none |
| | thaliana | glycoprotein 1 | | |
| AcrA | Candidalus | putative multidrug-efflux | CAF23274 | none |
| | Protochlamydia | transport protein acrA | | |
| | amoebophila | | | |
| | UWE2S | | | |
| AcrB | Candidatus | probable multidrug-efflux | CAF23275 | none |
| | Protochlantydia | transport protein, acrB | | |
| | amoebophila | | | |
| | UWE25 | | | |
| TolC | Francisella | Outer membrane protein [Cell | ABD59001 | none |
| | tularensis | envelope biogenesis, | | |
| | subsp. novicida | | | |
| AcrE | Shigella sonnei | transmembrane protein affects | YP_312213 | none |
| | Sv046 | septum formation and cell | | |
| | | membrane permeability | | |
| AcrF | E. coli | Acriflavine resistance protein F | P24181 | none |
| tl11619 | Thermo- | multidrug efflux transporter | NP_682409.1 | none |
| | synechococcus | | | |
| | elongatus | | | |
| | [BP-1] | | | |
| tl10139 | Thermo- | multidrug efflux transporter | NP_680930.1 | none |
| | synechococcus | | | |
| | elongatus | | | |
| | [BP-1] | | | |

8. Fermentation

| | | | | |
|---|---|---|---|---|
| replication | | | | |
| checkpoint | | | | |
| genes | | | | |
| timuD | Shigella sonnei | DNA polymerase V, subunit | YP_310132 | 3.4.21.- |
| | Ss046 | | | |
| umuC | E. coli | DNA polymerase V, subunit | ABC42261 | 2.7.7.7 |
| pntA, pntB | Shigella | NADH:NADPH | P07001, | 1.6.1.2 |
| | flexneri | transhydrogenase (alpha and | P0AB70 | |
| | | beta subunits) | | |

*see also section 2 enzymes - products having “:0” are unsaturated (no double bonds) and “:1” are saturated (1 double bond).

In some embodiments of the present invention, a wild-type gene encoding a protein comprises a polynucleotide sequence comprising an open reading frame (ORF) and a 5′ non-coding polynucleotide sequence (NC) comprising operably-linked regulatory sequences adjacent the 5′-end of the ORF that mediate the expression of the ORF and production of the encoded protein. The ORF has 5′ and 3′ ends, and in the wild-type gene the native operably-linked regulatory sequences are adjacent the 5′-end of the ORF; that is the operably-linked regulatory sequences that are natively adjacent the 5′-end of the ORF are the regulatory sequences known from the genomic sequence of the 5′-non-coding sequence of the wild-type gene. For example, in the a wild-type E. coli genome, native operably-linked regulatory sequences are those known to be adjacent the ORF (see, e.g., the complete genome sequence of Escherichia coli K-12; Blattner, F. R., et al., Science 277 (5331), 1453-1474 (1997); Riley, M., et al., Nucleic Acids Res. 34 (1), 1-9 (2006); Accession No. U00096.2). In some embodiments of the present invention, a variant ORF and/or a variant NC has less than 100% sequence identity to the wild-type ORF or the wild-type NC, respectively. Variant non-coding polynucleotide sequences can have from zero percent sequence identity to <100% percent sequence identity when compared to wild-type 5′ non-coding polynucleotide sequences comprising operably-linked regulatory sequences natively adjacent the 5′-end of the ORF in the wild-type gene; that is, the variant sequences are not the same as the native sequences.

In addition to the 5′ non-coding polynucleotide sequence comprising operably-linked regulatory sequences adjacent the 5′-end of an ORF, additional regulatory sequences can be modified generally following the methods described herein. Such additional regulatory sequences include, but are not limited to, 3′ non-coding polynucleotide sequences comprising operably-linked regulatory sequences adjacent the 3′-end of an ORF, or operably-linked regulatory sequences located in an intron polynucleotide sequence.

Methods of making the recombinant host cells and recombinant host cell cultures of the present invention are described in further detail herein.

### Methods of Making Recombinant Host Cells and Cultures

A fifth aspect of the present invention relates to methods of making the recombinant host cells and recombinant host cell cultures of the present invention. Recombinant host cells can be made, by the methods of the present invention, that produce compositions of fatty acid derivatives (e.g., fatty alcohols) having target aliphatic chain lengths. In this aspect, the methods generally comprise two core steps selected from the group consisting of step (A), step (B), and step (C), wherein the two steps are not the same step and the two steps are performed in any order to make the recombinant host cells; for example, step (A) followed by step (B), step (A) followed by step (C), step (B) followed by step (A), step (B) followed by step (C), step (C) followed by step (B), or step (C) followed by step (A).

In addition to these two core steps the method may comprises other steps, including, but not limited to, additional steps (A), (B), or (C), as well as other host cell manipulations (e.g., mutagenesis steps). Further, any step can be repeated, once or multiple times, as well as performed in any order (e.g., (A) followed by (A) followed by (B); (B) followed by (A) followed by (B); (A) followed by (B) followed by (A) followed by (B) followed by (C); and so on).

In the following descriptions of steps (A), (B), and (C), the starting polynucleotide can be, for example, a wild-type gene encoding the protein whose activity is being modified. In other embodiments, the starting polynucleotide sequence can be derived from such a wild-type gene (e.g., using a variant of the wild-type gene's polynucleotide sequence).

Step (A) generally comprises the following. A starting group of recombinant host cells is prepared using a starting polynucleotide sequence (SPS_{A}), the SPS_{A} comprising an open reading frame (ORF_{A}), the ORF_{A} having 5′ and 3′ ends, and a 5′ non-coding polynucleotide sequence (NC_{A}) comprising operably-linked regulatory sequences adjacent the 5′-end of the ORF_{A}. Each recombinant host cell comprises one or more variants of the SPS_{A}, wherein (i) the ORF_{A} encodes an elongation β-ketoacyl-ACP synthase protein, having an Enzyme Commission number of EC 2.3.1.-, and (ii) each variant SPS_{A} comprises a variant ORF_{A} and/or a variant NC_{A} having less than 100% sequence identity to the ORF_{A} or the NC_{A}, respectively.

Clones from the group of recombinant host cells are cultured in the presence of a carbon source. The clones are then screened to determine the aliphatic chain lengths of the fatty acid derivatives and the titer of the fatty acid derivatives produced by each clone. Among the clones, a clone is identified that produces a maximum titer of fatty acid derivatives having the target aliphatic chain length.

A clone (or one or more clones) from the group of recombinant host cells is selected that produces fatty acid derivatives having aliphatic chain lengths longer than the target aliphatic chain length at a titer less than the maximum titer (i.e., the maximum titer of the clone that was identified as producing the maximum titer of fatty acid derivatives having the target aliphatic chain length). The selected clone comprises a variant SPS_{A} (SPS_{VA}) comprising a variant ORF_{A} (ORF_{VA}) and/or a variant NC_{A} (NC_{VA}). In an alternative embodiment, for example when step (A) is the last step performed, the clone that was identified as producing the maximum titer of fatty acid derivatives having the target aliphatic chain length may be selected.

As noted above, the core two steps of the method can be performed in any order. Accordingly, (i) if step (A) is preceded in the method by step (B), then each recombinant host cell of the starting group for step (A) further comprises the SPS_{VB} (typically at least a variant ORF_{B} (ORF_{VB}) and/or a variant NC_{B} (NC_{VB})), or (ii) if step (A) is preceded in the method by step (C), then each recombinant host cell of the starting group for step (A) further comprises the SPS_{VC} (typically at least a variant ORF_{C} (ORF_{VC}) and/or a variant NC_{C} (NC_{VC})).

Step (B) general comprises the following. A starting group of recombinant host cells is prepared using a starting polynucleotide sequence (SPS_{B}), the SPS_{B} comprising an open reading frame (ORF_{B}), the ORF_{B} having 5′ and 3′ ends, and a 5′ non-coding polynucleotide sequence (NC_{B}) comprising operably-linked regulatory sequences adjacent the 5′-end of the ORF_{B}, each recombinant host cell comprising one or more variants of the SPS_{B}, wherein (i) the ORF_{B} encodes a thioesterase having an Enzyme Commission number of EC 3.1.1.5 or EC 3.1.2.-, and (ii) each variant SPS_{B} comprises a variant ORF_{B} and/or a variant NC_{B} having less than 100% sequence identity to the ORF_{B} or the NC_{B}, respectively.

Clones from the group of recombinant host cells are cultured in the presence of a carbon source. The clones are then screened to determine the aliphatic chain lengths of the fatty acid derivatives and the titer of the fatty acid derivatives produced by each clone. Among the clones, a clone is identified that produces a maximum titer of fatty acid derivatives having the target aliphatic chain length.

A clone (or one or more clones) from the group of recombinant host cells is selected that produces fatty acid derivatives having the target aliphatic chain length at a titer approximately equal to the maximum titer (i.e., the maximum titer of the clone that was identified as producing the maximum titer of fatty acid derivatives having the target aliphatic chain length). The selected clone comprises a variant SPS_{B} (SPS_{VB}) comprising a variant ORF_{B} (ORF_{VB}) and/or a variant NC_{B} (NC_{VB}).

Typically, the selected clone that produces fatty acid derivatives having the target aliphatic chain lengths produces the fatty acid derivatives at a titer approximately equal to the maximum titer. In other embodiments of the methods of the present invention the selected clone produces the fatty acid derivatives having the target aliphatic chain lengths at a titer within about 2% of the maximum titer, within about 5% of the maximum titer, within about 10% of the maximum titer, within about 20% of the maximum titer, or within about 30% of the maximum titer.

As noted above, the core two steps of the method can be performed in any order. Accordingly, (i) if step (B) is preceded in the method by step (A), then the each recombinant host cell of the starting group for step (B) further comprises the SPS_{VA}, (typically at least a variant ORF_{A} (ORF_{VA}) and/or a variant NC_{A} (NC_{VA})), or (ii) if step (B) is preceded in the method by step (C), then each recombinant host cell of the starting group for step (B) further comprises the SPS_{VC} (typically at least a variant ORF_{C} (ORF_{VC}) and/or a variant NC_{C} (NC_{VC})).

Step (C) generally comprises the following. A starting group of recombinant host cells is prepared using a starting polynucleotide sequence (SPS_{C}), the SPS_{C} comprising an open reading frame (ORF_{C}), the ORF_{C} having 5′ and 3′ ends, and a 5′ non-coding polynucleotide sequence (NC_{C}) comprising operably-linked regulatory sequences adjacent the 5′-end of the ORF_{C}. Each recombinant host cell comprises one or more variants of the SPS_{C}, wherein (i) the ORF_{C} encodes a β-hydroxyacyl-ACP dehydratase protein, having an Enzyme Commission number of EC 4.2.1.- or 4.2.1.60, and (ii) each variant SPS_{C} comprises a variant ORF_{C} and/or a variant NC_{C} having less than 100% sequence identity to the ORF_{C} or the NC_{C}, respectively.

Clones from the group of recombinant host-cells are cultured in the presence of a carbon source. The clones are then screened to determine the aliphatic chain lengths of the fatty acid derivatives, percent saturation of the aliphatic chains of the fatty acid derivatives, and the titer of the fatty acid derivatives for each clone. Among the clones, a clone is identified that produces a maximum titer of fatty acid derivatives having the target aliphatic chain length and a preferred percent saturation; and

A clone (or one or more clones) from the group of recombinant host cells is selected that produces fatty acid derivatives having the target aliphatic chain length and the preferred percent saturation at a titer approximately equal to the maximum titer, wherein the selected clone comprises a variant SPS_{C} (SPS_{VC}) comprising a variant ORF_{C} (ORF_{VC}) and/or a variant NC_{C} (NC_{VC}). In other embodiments of the methods of the present invention the selected clone produces the fatty acid derivatives having the target aliphatic chain lengths at a titer within about 2% of the maximum titer, within about 5% of the maximum titer, within about 10% of the maximum titer, within about 20% of the maximum titer, or within about 30% of the maximum titer.

As noted above, the core two steps of the method can be performed in any order. Accordingly, (i) if step (C) is preceded in the method by step (B), then each recombinant host cell of the starting group for step (C) further comprises the SPS_{VB} (typically at least a variant ORF_{B} (ORF_{VB}) and/or a variant NC_{B} (NC_{VB})), or (ii) if step (C) is preceded in the method by step (A), then the each recombinant host cell of the starting group for step (C) further comprises the SPS_{VA}, (typically at least a variant ORF_{A} (ORF_{VA}) and/or a variant NC_{A} (NC_{VA})).

In some embodiments of the methods of the present invention, the composition of fatty acid derivatives having the target aliphatic chain length further has a preferred percent saturation. For example, the composition of fatty acid derivatives having the target aliphatic chain length comprise saturated and unsaturated aliphatic chains, and typically the preferred percent saturation of the aliphatic chains of the fatty acid derivative is about 90% or greater of the target fatty acid derivatives having saturated aliphatic chains. However, following the methods of the present invention, one of ordinary skill in the art can select a preferred percent saturation of any value, for example, a preferred percent saturation of about 5% (i.e., about 95% of the aliphatic chains are unsaturated), a preferred percent saturation of about 60% (i.e., about 40% of the aliphatic chains are unsaturated), and so on.

Step (A) is typically used for optimization of production of the fatty acid derivatives having the target aliphatic chain lengths. Step (B) is typically used for optimization of the titer of the fatty acid derivatives having the target aliphatic chain lengths and/or preferred percent saturation. Step (C) is typically used for optimization of production of the fatty acid derivatives having the target aliphatic chain lengths and a preferred percent saturation. In an alternative embodiment of step (C), a starting group of recombinant host cells is prepared using a starting polynucleotide sequence (SPS_{F}), the SPS_{F} comprising an open reading frame (ORF_{F}), the ORF_{F} having 5′ and 3′ ends, and a 5′ non-coding polynucleotide sequence (NC_{F}) comprising operably-linked regulatory sequences adjacent the 5′-end of the ORF_{F}. Each recombinant host cell comprises one or more variants of the SPS_{F}, wherein (i) the ORF_{F} encodes a β-ketoacyl-ACP synthase protein, for example, an 3-oxoacyl-[acyl-carrier-protein] synthase I protein, having an Enzyme Commission number of EC 2.3.1.41, and (ii) each variant SPS comprises a variant ORF_{F} and/or a variant NC_{F} having less than 100% sequence identity to the ORF_{F} or the NC_{F}, respectively. Culturing, screening, and selection are carried out as described above for step (C).

Total fatty acid derivative titer, titers of fatty acid derivatives having different aliphatic chain lengths, and percent saturation of the aliphatic chains of the fatty acid derivatives can be determined by a number of methods (see, e.g., U.S. Patent Publication No. 20100251601, published 7 Oct. 2010) known to those of ordinary skill in the art, for example, thin-layer chromatography (TLC), high-performance liquid chromatography (HPLC), gas chromatography/flame ionization detection (GC/FID), gas chromatography/mass spectroscopy (GC/MS), liquid chromatography/mass spectroscopy (LC/MS), and mass spectroscopy (MS).

In one embodiment of the present invention, a ratio (C_{X}/C_{Y}) of two selected aliphatic chain lengths is used to characterize the aliphatic chain lengths and the target aliphatic chain lengths, the C_{X}/C_{Y} ratio being the titer of the fatty acid derivative having an aliphatic chain length of C_{X} to the titer of the fatty acid derivative having an aliphatic chain length of C_{Y}, where X and Y are integer values and X is less than Y.

In some embodiments of the methods the present invention, the fatty acid derivatives having target aliphatic chain lengths can be fatty acid derivatives having aliphatic chain lengths selected from the group of aliphatic chains lengths consisting of between C₈, C₁₀, C₁₂, C₁₄, C₁₆, C₁₈, C₂₀, and combinations thereof. The target fatty acid derivatives can be, for example, fatty acid derivatives having aliphatic chain lengths of C₈, fatty acid derivatives having aliphatic chain lengths of C₁₀, fatty acid derivatives having aliphatic chain lengths of C₁₂, fatty acid derivatives having aliphatic chain lengths of C₁₄, fatty acid derivatives having aliphatic chain lengths of C₁₆, fatty acid derivatives having aliphatic chain lengths of C₁₈, fatty acid derivatives having aliphatic chain lengths of C₂₀, as well as combinations thereof. In one embodiment, a ratio (C_{X}/C_{Y}) of two selected aliphatic chain lengths is used to characterize the aliphatic chain length. The C_{X}/C_{Y} ratio is the titer of fatty acid derivatives having an aliphatic chain length of C_{X} to the titer of fatty acid derivatives having an aliphatic chain length of C_{Y}. In some embodiments of the present invention, C_{X}/C_{Y} has a value of between about 1.5 to about 6, where X and Y are integer values and X is less than Y. In other embodiments of the present invention, C_{X}/C_{Y} has a value of at least about 2, where X and Y are integer values and X is less than Y. In a preferred embodiment, C_{X}/C_{Y} has a value of between about 2 and about 4, where X and Y are integer values and X is less than Y. Examples of X and Y values include, but are not limited to: X=8, Y=10; X=12, Y=14; X=14, Y=16; and X=18, Y=20. Other combinations of X and Y values are readily apparent to one of ordinary skill in the art in view of the teachings of the present specification.

Creating variant polynucleotide sequences can be carried out by methods known to those of ordinary skill in the art, in view of the teachings of the present-specification. Typically, variant polynucleotide sequences are produced by mutagenesis that results in one or more mutations in the gene including, but not limited to, one or more mutations in: a polynucleotide sequence encoding a promoter sequence (e.g., an RNA polymerase binding site); a polynucleotide sequence encoding a translational control sequence (e.g., a ribosome binding site or translation initiation site); a polynucleotide sequence encoding the open reading frame that encodes the protein; and combinations thereof. Exemplary mutagenesis methods are described below.

In some embodiments of the methods of the present invention, the variant NC_{VZ}, where Z=A, B, or C, (i.e., variant 5′ non-coding polynucleotide sequence) is obtained from a library generated by randomization of the NC_{VZ}. The non-coding polynucleotide sequences that can be randomized include, but are not limited to, promoter sequences, translational control sequences (e.g., ribosome binding sites), enhancer sequences, and binding sites for gene activators or repressors.

In some embodiments of the methods of the present invention, the variant ORF_{VZ}, where Z=A, B, or C, (i.e., the protein coding open reading frame of the polynucleotide sequence) is obtained by mutagenesis of the ORF_{VZ}.

In some embodiments of the methods of the present invention, the ORF_{A} encoding the elongation β-ketoacyl-ACP synthase protein encodes a 3-oxoacyl-[acyl-carrier-protein] synthase I protein (Enzyme Commission number EC 2.3.1.41) or a 3-oxoacyl-[acyl-carrier-protein] synthase 11 protein (Enzyme Commission number EC 2.3.1.179). In preferred embodiments using 3-oxoacyl-[acyl-carrier-protein] synthase I protein, the synthase protein ORF_{A} encodes an E. coli fabB derived 3-oxoacyl-[acyl-carrier-protein] synthase I protein that has the sequence set forth in SEQ ID NO:2, and the variant synthase protein ORF_{A} encodes a 3-oxoacyl-[acyl-carrier-protein] synthase I protein that has at least about 70%, about 75%, about 80%, about 85%, preferably about 90% or about 95% or greater sequence identity to the E. coli fabB protein (SEQ ID NO:2). In preferred embodiments using 3-oxoacyl-[acyl-carrier-protein] synthase II protein, the synthase protein ORF_{A} encodes an E. coli fabF derived 3-oxoacyl-[acyl-carrier-protein] synthase II protein that has the sequence set forth in SEQ ID NO:4, and the variant synthase protein ORF_{A} encodes a 3-oxoacyl-[acyl-carrier-protein] synthase II protein that has at least about 70%, about 75%, about 80%, about 85%, preferably about 90% or about 95% or greater sequence identity to the E. coli fabF protein (SEQ ID NO:4). Further, a variant 5′ non-coding polynucleotide sequence, variant NC_{A}, can be provided, for example, from a library generated by randomization of the NC_{A}. Variant non-coding polynucleotide sequences (e.g., variant NC_{A}) typically have from zero percent sequence identity to <100% percent sequence identity when compared to the starting non-coding polynucleotide sequences (e.g., NC_{A}).

In some embodiments of the methods of the present invention, the ORF_{B} encoding the thioesterase include, but are not limited to, sequences encoding a thioesterase protein (Enzyme Commission numbers of EC 3.1.1.5 or EC 3.1.2.-). In preferred embodiments using the thioesterase protein, the thioesterase protein ORF_{B} encodes an E. coli tesA derived thioesterase protein that has the sequence set forth in SEQ ID NO:6, SEQ ID NO:8, SEQ ID NO:17, or SEQ ID NO:19, and the variant ORF_{B} encodes a thioesterase protein that has at least about 70%, about 75%, about 80%, about 85%, preferably about 90% or about 95% or greater sequence identity to the E. coli tesA protein (SEQ ID NO:6, SEQ ID NO:8, SEQ ID NO:17, or SEQ ID NO:19, respectively). Further, a variant 5′ non-coding polynucleotide sequence, variant NC_{B}, can be provided, for example, from a library generated by randomization of the NC_{B}. Variant non-coding polynucleotide sequences (e.g., variant NC_{B}) typically have from zero percent sequence identity to <100% percent sequence identity when compared to the starting non-coding polynucleotide sequences (e.g., NC_{B}).

In some embodiments of the methods of the present invention, the ORF_{C} encoding the β-hydroxyacyl-ACP dehydratase protein encodes a protein having an Enzyme Commission number of EC 4.2.1.- or EC 4.2.1.60. In preferred embodiments, the ORF_{C} encodes an E. coli fabZ derived (3R)-hydroxymyristol acyl carrier protein dehydratase protein that has the sequence set forth in SEQ ID NO:14, and the variant ORF_{C} encodes a (3R)-hydroxymyristol acyl carrier protein dehydratase protein that has at least about 70%, about 75%, about 80%, about 85%, preferably about 90% or about 95% or greater sequence identity to the E. coli fabZ protein (SEQ ID NO:14). In some embodiments, the ORF_{C} encodes an E. coli fabA derived β-hydroxydecanoyl thioester dehydratase/isomerase protein that has the sequence set forth in SEQ ID NO:12, and the variant ORF_{C} encodes a β-hydroxydecanoyl thioester dehydratase/isomerase protein that has at least about 70%, about 75%, about 80%, about 85%, preferably about 90% or about 95% or greater sequence identity to an E. coli fabA protein (SEQ ID NO:12).

Further, a variant 5′ non-coding polynucleotide sequence, variant NC_{C}, can be provided, for example, from a library generated by randomization of the NC_{C}. Variant non-coding polynucleotide sequences (e.g., variant NC_{C}) typically have from zero percent sequence identity to <100% percent sequence identity when compared to the starting non-coding polynucleotide sequences (e.g., NC_{C}).

Recombinant host cells made by the methods of the present invention can further comprise one or more nucleotide sequence encoding a carboxylic acid reductase protein that has an Enzyme Commission number of EC 6.2.1.3 or EC 1.2.1.42, and operably-linked regulatory sequences. In some embodiments, the carboxylic acid reductase protein is a protein that has at least about 70%, about 75%, about 80%, about 85%, preferably about 90% or about 95% or greater sequence identity to a Mycobacterium smegmatis carB fatty acid reductase protein (SEQ ID NO:10). In other embodiments, the carboxylic acid reductase protein is a protein that has at least about 70%, about 75%, about 80%, about 85%, preferably about 90% or about 95% or greater sequence identity to (i) a Mycobacterium tuberculosis fadD9 protein (SEQ ID NO:21; see, also, US Patent Publication No. 20100105963), or (ii) a Mycobacterium smegmatis carA protein (SEQ ID NO:23; see, also, US Patent Publication No. 20100105963).

In addition, the recombinant host cells made by the methods of the present invention can further comprise one or more polynucleotide sequences encoding an alcohol dehydrogenase protein having an Enzyme Commission number of EC 1.1.-.-, EC 1.1.1.1, or EC 1.2.1.10, and operably-linked regulatory sequences. Examples of such alcohol dehydrogenase proteins include, but are not limited to, E. coli AdhE, aldehyde-alcohol dehydrogenase protein, or E. coli yqhD, alcohol dehydrogenase protein.

Embodiments of the recombinant host cells made by the methods of present invention can further comprise one or more polynucleotide sequence encoding one or more additional proteins and operably-linked regulatory sequences. Examples of such additional proteins include, but are not limited to, acetyl-CoA acetyltransferase; β-hydroxybutyryl-CoA dehydrogenase; crotonase butyryl-CoA dehydryogenase; and coenzyme A-acylating aldehyde dehydrogenase. Such additional proteins can be expressed in the recombinant host cells to facilitate production of particular fatty acid derivatives from acyl-ACPs as substrates (see, e.g., FIG. 2 and Table 1).

In some embodiments of the methods of the present invention, the operably-linked regulatory sequences can confer constitutive expression or regulatable expression of the operably-linked open reading frame; resulting in constitutive or regulatable expression of the protein encoded by the open reading frame. For example, the expression of a protein in a host cell can be mediated via a constitutive promoter, or via an inducible/repressible promoter. Examples of inducible/repressible promoters are known in the art and include, but are not limited to, the following: the E. coli lac operon promoter; and Saccharomyces cerevisiae GAL4-inducible promoters.

The one or more polynucleotide sequences, comprising open reading frames encoding proteins and operably-linked regulatory sequences can be integrated into a chromosome of the recombinant host cells, incorporated in one or more plasmid expression system resident in the recombinant host cells, or both. In the Examples, plasmid expression systems are used to illustrate embodiments of the present invention.

In the method steps (A), (B), and (C), as described herein, use of subscripts is used to simplify description of the steps, for example, an “SPS_{A},” an “SPS_{B},” an “SPS_{C},” a “selected clone comprises a variant SPS_{A} (SPS_{VA}) comprising a variant ORF_{A} (ORF_{VA}) and/or a variant NC_{A} (NC_{VA}),” a “selected clone comprises a variant SPS_{B} (SPS_{VB}) comprising a variant ORF_{B} (ORF_{VB}) and/or a variant NC_{B} (NC_{VB}),” and a “selected clone comprises a variant SPS_{C} (SPS_{VC}) comprising a variant ORF_{C} (ORF_{VC}) and/or a variant NC_{C} (NC_{VC}).” The use of such subscripts in the description of the steps is not intended to be limiting. Regarding the order in which the steps can be performed, one of ordinary skill in the art can suitably modify the step in view of the teachings of the present specification, for example, as follows. When any step precedes a particular method step (A), (B), or (C), “preparing a starting group of recombinant host cells” for the step (A), (B), or (C) typically includes moving forward one or more variant polynucleotide sequences from the preceding step that is used when preparing the starting group of recombinant host cells in following particular method step (A), (B), or (C).

Recombinant host cells can be made, by the methods of the present invention, that produce compositions of fatty acid derivatives (e.g., fatty alcohols) having target aliphatic chain lengths. The method typically comprises two core steps selected from the group consisting of step (A), step (B), and step (C), wherein the two steps are not the same step and the two steps are performed in any order to make the recombinant host cells; for example, step (A) followed by step (B), step (A) followed by step (C), step (B) followed by step (A), step (B) followed by step (C), step (C) followed by step (B), or step (C) followed by step (A).

In one embodiment of the methods of the present invention, the composition of fatty acid derivatives having the target aliphatic chain length is a composition of fatty alcohols having the target aliphatic chain length.

In one embodiment of the present invention, culturing the recombinant host cells made by the methods of the present invention in the presence of a carbon source produces a fatty acid derivative compositor having the target aliphatic chain length and a titer of from 30 g/L to 250 g/L of the composition of.

In a further embodiment of the present invention, culturing the recombinant host cells made by the methods of the present invention in the presence of a carbon source produces a yield of from 10% to 40% of the composition of fatty acid derivatives having the target aliphatic chain length.

In another embodiment of the present invention, culturing the recombinant host cells made by the methods of the present invention in the presence of a carbon source provides a productivity of 700 mg/L/hour to 3000 mg/L/hour of the composition of fatty acid derivatives having the target aliphatic chain length.

The recombinant host cells of the present invention, and cultures thereof, can be mammalian cells, plant cells, insect cells, algal cells, fungus cells, or bacterial cells. In one embodiment, the recombinant host cell is a microorganism (e.g., bacteria or fungi). In preferred embodiments, the recombinant host cells are bacteria. In a preferred embodiment, the bacteria are Escherichia coli.

The present invention includes recombinant host cells (e.g., recombinant microorganisms) made by the methods of the present invention, as well as cultures of the recombinant host cells. Such recombinant host cells typically produce fatty acid derivatives having target aliphatic chain lengths and/or a fatty acid derivative having aliphatic chains of preferred saturation.

### Methods of Mutagenesis for Making Variant Polynucleotide Sequences

In aspects of the methods of the present invention, mutagenesis is used to prepare groups of recombinant host cells for screening. Typically, the recombinant host cells comprise one or more polynucleotide sequences that include an open reading for a protein, as well as operably-linked regulatory sequences. Numerous examples of proteins useful in the practice of the methods of the present invention are described herein and include, but are not limited to, an elongation β-ketoacyl-ACP synthase protein, a thioesterase, a β-hydroxyacyl-ACP dehydratase protein, and a carboxylic acid reductase protein. Examples of regulatory sequences useful in the practice of the methods of the present invention are also described herein, for example, RNA promoter sequences, transcription factor binding sequences, transcription termination sequences, modulators of transcription, nucleotide sequences that affect RNA stability, and translational regulatory sequences. Mutagenesis of such polynucleotide sequences can be performed using genetic engineering techniques, such as site directed mutagenesis, random chemical mutagenesis, Exonuclease III deletion procedures, or standard cloning techniques. Alternatively, mutations in polynucleotide sequences can be created using chemical synthesis or modification procedures.

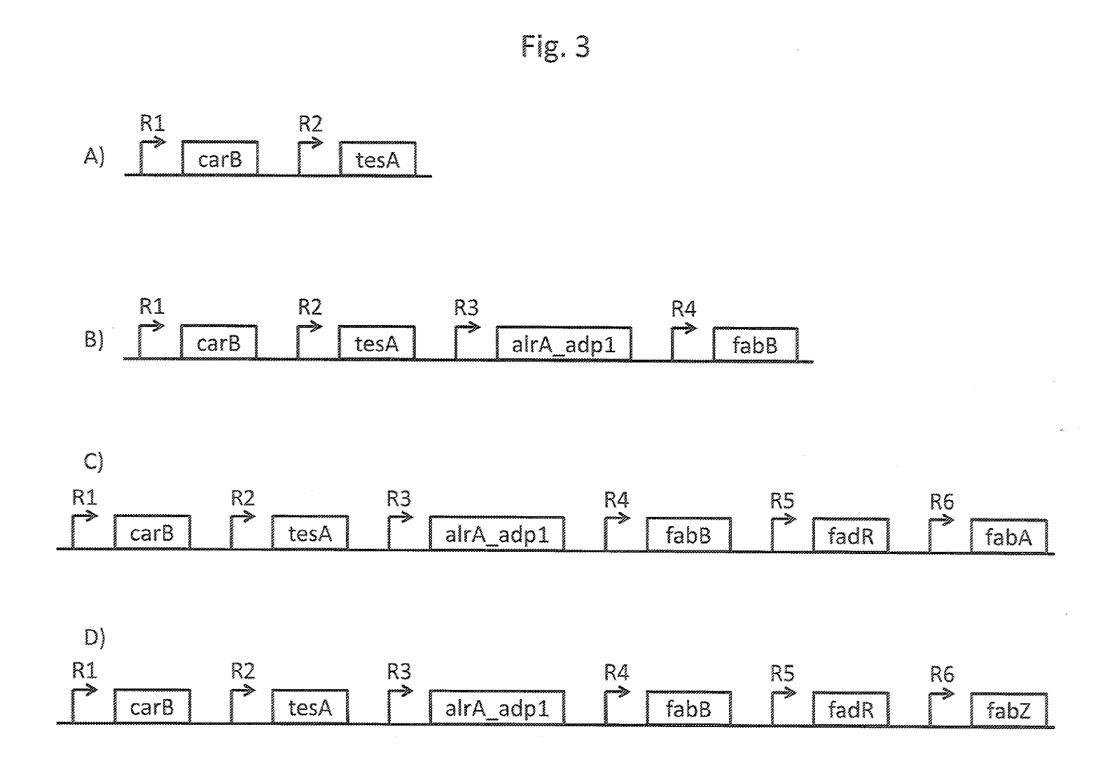

Mutagenesis methods are well known in the art and include, for example, the following. Error prone PCR (see, e.g., Leung et al., Technique 1:11-15, 1989; and Caldwell et al., PCR Methods Applic. 2:28-33, 1992), PCR is performed under conditions where the copying fidelity of the DNA polymerase is low, such that a high rate of point mutations is obtained along the entire length of the PCR product. Briefly, in such procedures, polynucleotides to be mutagenized (e.g., regulatory sequences, such as R2, R4, and R6 of FIG. 3; or polynucleotides comprising open reading frames encoding proteins, such as car, tesA, fabB, fabF, fabA, and fabZ) are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized, 30 pmole of each PCR primer, a reaction buffer comprising 50 mM KCl, 10 mM Tris HCl (pH 8.3), and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min., 45° C. for 1 min., and 72° C. for 1 min. It will be appreciated that these parameters can be varied as appropriate. The mutagenized polynucleotides are then cloned into an appropriate vector and the activities of the affected polypeptides encoded by the mutagenized are evaluated.

Mutagenesis can also be performed using oligonucleotide directed mutagenesis (see, e.g., Reidhaar-Olson et al., Science 241:53-57, 1988) to generate site-specific mutations in any cloned DNA of interest. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered, and the activities of affected polypeptides are assessed.

Another mutagenesis method for generating polynucleotide sequence variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408.

Still another mutagenesis method of generating polynucleotide sequence variants is sexual PCR Mutagenesis (Stemmer, PNAS, USA 91:10747-10751, 1994). In sexual PCR mutagenesis, forced homologous recombination occurs between DNA molecules of different, but highly related, DNA sequence in vitro as a result of random fragmentation of the DNA molecule based on sequence homology. This is followed by fixation of the crossover by primer extension in a PCR reaction.

Polynucleotide sequence variants can also be created by in vivo mutagenesis. In some embodiments, random mutations in a nucleic acid sequence are generated by propagating the polynucleotide sequence in a bacterial strain, such as an E. coli strain, which carries mutations in one or more of the DNA repair pathways. Such “mutator” strains have a higher random mutation rate than that of a wild-type strain. Propagating a DNA sequence in one of these strains will eventually generate random mutations within the DNA. Mutator strains suitable for use for in vivo mutagenesis are described in, for example, PCT International Publication No. WO 91/16427.

Polynucleotide sequence variants can also be generated using cassette mutagenesis. In cassette mutagenesis, a small region of a double stranded DNA molecule is replaced with a synthetic oligonucleotide “cassette” that differs from the starting polynucleotide sequence. The oligonucleotide often contains completely and/or partially randomized versions of the starting polynucleotide sequence. There are many applications of cassette mutagenesis; for example, preparing mutant proteins by cassette mutagenesis (see, e.g., Richards, J. H., Nature 323, 187 (1986); Ecker, D. J., et al., J. Biol. Chem. 262:3524-3527 (1987)); codon cassette mutagenesis to insert or replace individual codons (see, e.g., Kegler-Ebo, D. M., et al., Nucleic Acids Res. 22(9): 1593-1599 (1994)); preparing variant polynucleotide sequences by randomization of non-coding polynucleotide sequences comprising regulatory sequences (e.g., ribosome binding sites, see, e.g., Barrick, D., et al., Nucleic Acids Res. 22(7): 1287-1295 (1994); Wilson, B. S., et al., Biotechniques 17:944-953 (1994)).

Recursive ensemble mutagenesis (see, e.g., Arkin et al., PNAS, USA 89:7811-7815, 1992) can also be used to generate polynucleotide sequence variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis.

Exponential ensemble mutagenesis (see, e.g., Delegrave et al., Biotech. Res. 11:1548-1552, 1993) can also be used to generate polynucleotide sequence variants. Exponential ensemble mutagenesis is a process for generating combinatorial libraries with a high percentage of unique and functional mutants, wherein small groups of residues are randomized in parallel to identify, at each altered position, amino acids which lead to functional proteins. Random and site-directed mutagenesis can also be used (see, e.g., Arnold, Curr. Opin. Biotech. 4:450-455, 1993).

Further, standard methods of in vivo mutagenesis can be used. For example, host cells, comprising one or more polynucleotide sequences that include an open reading frame for a protein, as well as operably-linked regulatory sequences, can be subject to mutagenesis via exposure to radiation (e.g., UV light or X-rays) or exposure to chemicals (e.g., ethylating agents, alkylating agents, or nucleic acid analogs). In some host cell types, for example, bacteria, yeast, and plants, transposable elements can also be used for in vivo mutagenesis.

In aspects of the methods of the present invention that use mutagenesis of one or more polynucleotide sequences, the resulting expressed protein product typically retains the same biological function even though the protein demonstrates a modified activity of the biological function. For example, when preparing a group of recombinant microorganisms by mutagenesis of one or more polynucleotide sequences including (i) the open reading frame encoding E. coli tesA thioesterase protein, and (ii) operably-linked regulatory sequences, the protein expressed from the resulting mutagenized polynucleotide sequences maintains the thioesterase biological function but a modified activity of the thioesterase is observed in the recombinant microorganism.

In aspects of the methods of the present invention, differences in activity are determined between a recombinant host cell and a corresponding wild-type host cell. For example, one or more starting polynucleotide sequences including an open reading frame encoding a protein and operably-linked regulatory sequences are subjected to mutagenesis (i.e., “starting” polynucleotide sequences are the polynucleotide sequences to be mutagenized, and give rise to “mutagenized” polynucleotide sequences). The activity of the protein in a recombinant host cell comprising the one or more mutagenized polynucleotide sequences is compared to the activity of the protein in a corresponding wild-type host cell comprising the one or more starting polynucleotide sequences. As an illustration, in an embodiment of method step (B), as described herein, a group of recombinant microorganisms is prepared, these recombinant microorganisms comprises one or more polynucleotide sequences including an open reading frame encoding a thioesterase and operably-linked to regulatory sequences, wherein the activity of the thioesterase in the recombinant microorganism is modified. Mutagenesis of one or more starting polynucleotide sequences including the open reading frame encoding the thioesterase and operably-linked regulatory sequences is used to preparing the group of recombinant microorganisms. The activity of the thioesterase in recombinant microorganisms comprising the one or more mutagenized polynucleotide sequences is compared to the activity of the thioesterase in a corresponding wild-type microorganism comprising the one or more starting polynucleotide sequences.

In one embodiment of the methods of the present invention, the modified activity of a protein can be determined as follows. Recombinant host cells (comprising one or more mutagenized polynucleotide sequences encoding the protein) are cultured and screened to identify characteristics of fatty acid derivatives produced by the recombinant host cells; for example, aliphatic chain lengths of a fatty acid derivative, titer of a fatty acid derivative, yield of a fatty acid derivative, productivity of a fatty acid derivative, saturation of the aliphatic chains of a fatty acid derivative, as well as combinations thereof. A modified activity of the protein is determined by comparison of the same characteristic(s) of fatty acid derivatives produced by a corresponding wild-type host cell (comprising one or more starting polynucleotide sequences encoding the protein) and identification. Of differences in the characteristics.

In view of the teachings of the present specification, the EC designations and the enzymatic activities for proteins involved in fatty acid biosynthesis (as described herein), and the structure/function information, available these proteins, one of ordinary skill in the art has sufficient guidance in view of the teachings of the specification to perform mutagenesis of coding sequences to obtain proteins having modified activities.

### Genetic Engineering of Host Cells to Make Recombinant Host Cells

Various recombinant host cells can be used to produce fatty acid derivatives, as described herein. A host cell can be any prokaryotic or eukaryotic cell. For example, a gene encoding a polypeptide described herein (e.g., an elongation β-ketoacyl-ACP synthase protein, a thioesterase, a β-hydroxyacyl-ACP dehydratase protein, and/or a carboxylic acid reductase protein) can be expressed in bacterial cells (e.g., E. coli), insect cells, algae, yeast, or mammalian cells (e.g., Chinese hamster ovary cells (CHO) cells, COS cells, VERO cells, BHK cells, HeLa cells, Cv1 cells, MDCK cells, 293 cells, 3T3 cells, or PC12 cells). Other exemplary host cells were described above. In a preferred embodiment, the host cell is an E. coli cell, a Saccharomyces cerevisiae cell, or a Bacillus subtilis cell. In a more preferred embodiment, the host cell is from E. coli strains B, C, K, or W. Other suitable host cells are known to those skilled in the art.

Additional host cells that can be used in the methods described herein are described in Published U.S. Patent Application Nos. 20110008861 and 20090275097.

Various methods well known in the art can be used to genetically engineer host cells to provide recombinant cells. The methods can include the use of vectors, preferably expression vectors, containing coding sequences for the proteins described herein.

Recombinant expression vectors for use in the present invention may comprise one or polynucleotide sequences encoding proteins as well as operably-linked regulatory sequences suitable to provide expression of the encoded proteins in a host cell. The recombinant expression vectors can include one or more regulatory sequences, selected on the basis of the host cell to be used for expression. Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors described herein can be introduced into host cells to produce polypeptides encoded by the nucleic acids as described herein.

Expression of genes encoding polypeptides in prokaryotes, for example, E. coli, is most often carried out with vectors containing constitutive or inducible promoters directing the expression of polypeptides. Fusion vectors can add a number of amino acids to a polypeptide encoded therein, usually to the amino terminus of the recombinant polypeptide. Such fusion vectors can, for example, provide an initiating ATG for sequences lacking such an initiation codon.

Examples of inducible E. coli expression vectors include pTrc (Amann et al., Gene (1988) 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gene 10-lac fusion promoter mediated by a co-expressed T7 viral RNA polymerase (T7 gn 1). This viral polymerase is supplied, for example, by host strains BL21(DE3) or HMS174(DE3) from a resident lambda pro-phage harboring a T7 gn1 gene under the transcriptional control of the lacUV-5 promoter.

In another embodiment, the host cell is a yeast cell. In this embodiment, the expression vector is a yeast expression vector. Examples of vectors for expression in yeast S. cerevisiae include pYepSec1 (Baldari et al., EMBO J. (1987) 6:229-234), pMFa (Kurjan et al., Cell (1982) 30:933-943), pJRY88 (Schultz et al., Gene (1987) 54:113-123), pYES2 (Invitrogen Corporation, Carlsbad, Calif.), and picZ (Invitrogen Corp, Carlsbad, Calif.).

In another embodiment, a protein described herein can be expressed in insect cells using baculovirus expression vectors. Baculovirus vectors available for expression of proteins in cultured insect cells (e.g., Sf9 cells) include, for example, the pAc series (Smith et al., Mol. Cell Biol. (1983) 3:2156-2165) and the pVL series (Lucklow et al., Virology (1989) 170:31-39).

In yet another embodiment, the nucleic acids described herein can be expressed in mammalian cells using a mammalian expression vector. Examples of mammalian expression vectors include pCDM8 (Seed, Nature (1987) 329:840) and pMT2PC (Kaufman et al., EMBO J. (1987) 6:187-195). When used in mammalian cells, the expression vector's control functions can be provided by viral regulatory elements. For example, commonly used promoters are derived from polyoma, Adenovirus type 2, cytomegalovirus, and Simian Virus 40. Other suitable expression systems for both prokaryotic and eukaryotic cells have been described (see, e.g., Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

Vectors can be introduced into prokaryotic or eukaryotic cells via conventional transformation or transfection techniques including, but not limited to a variety of art-recognized techniques for introducing nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to antibiotics) can be introduced into the host cells along with the gene of interest. Selectable markers include those that confer resistance to drugs, such as ampicillin, kanamycin, chloramphenicol, spectinomycin, or tetracycline. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

In addition to extra-chromosomal expression vectors (such as, plasmids), polynucleotide expression vectors can be integrated into a host cell's genome following standard techniques, for example, via homologous recombination and integration.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) can be introduced into the host cells along with the gene of interest. Preferred selectable markers include those that confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by drug selection.

### Further Aspects of the Present Invention

Further aspects of the present invention include the following: In a sixth aspect the present invention relates more specifically to methods of making the recombinant host cells and recombinant host cell that produce compositions of fatty acid derivatives having target aliphatic chain lengths.

These recombinant host cells typically have a modified activity of a β-hydroxyacyl-ACP dehydratase protein, having an Enzyme Commission number of EC 4.2.1.- or 4.2.1.60. The methods of the present invention used to make these recombinant host cells typically use at least step (C) or a variation of step (A), wherein the starting polynucleotide sequence (SPS) comprises an open reading frame polynucleotide sequence (ORF) encoding the β-hydroxyacyl-ACP dehydratase protein, the ORF having 5′ and 3′ ends, and a 5′ non-coding polynucleotide sequence (NC) comprising operably-linked regulatory sequences adjacent the 5′-end of the ORF. The recombinant host cells comprise one or more variants of the SPS, encoding the β-hydroxyacyl-ACP dehydratase protein and operably-linked regulatory sequences, comprising a variant ORF and/or a variant NC having less than 100% sequence identity to the ORF or the NC, respectively. The step (C) or variation of step (A) can be followed, for example, by step (B) if further optimization of the titer of the fatty acid derivatives having the target aliphatic chain lengths is needed or desired.

In a seventh aspect the present invention relates more specifically to methods of making the recombinant host cells and recombinant host cell that produce compositions of fatty acid derivatives having preferred percent saturation. These recombinant host cells typically have a modified activity of a β-hydroxyacyl-ACP dehydratase protein that lacks isomerase activity, having an Enzyme Commission number of EC 4.2.1.-. The methods of the present invention used to make these recombinant host cells typically use at least step (C) or a variation of step (A), wherein the starting polynucleotide sequence (SPS) comprises an open reading frame polynucleotide sequence (ORF) encoding the β-hydroxyacyl-ACP dehydratase protein that lacks isomerase activity, the ORF having 5′ and 3′ ends, and a 5′ non-coding polynucleotide sequence (NC) comprising operably-linked regulatory sequences adjacent the 5′-end of the ORF. The recombinant host cells comprise one or more variants of the SPS, encoding the β-hydroxyacyl-ACP dehydratase protein that lacks isomerase activity and operably-linked regulatory sequences, comprising a variant ORF and/or a variant NC having less than 100% sequence identity to the ORF or the NC, respectively. The step (C) or variation of step (A) can be followed by, for example, step (B) if further optimization of the titer of the fatty acid derivatives having the preferred percent saturation is needed or desired.

In an eighth aspect, the present invention relates more specifically to a method of producing a composition of fatty acid derivatives having a target aliphatic chain length and/or preferred degree of saturation. The method typically comprises culturing, in the presence of a carbon source, a recombinant host cell as described herein. In one embodiment of this method, the culturing comprises fermentation. In a preferred embodiment, fermentation is used and the method further comprises substantial purification of the fatty acid derivatives.

In a ninth aspect, the present invention relates to substantially purified compositions of fatty acid derivatives (e.g., fatty alcohols) produced using the recombinant host cell cultures of the present invention.

### Fermentation Production and Isolation of Fatty Acid Derivatives

Production and isolation of fatty acid derivatives using the recombinant host cell cultures described herein, can be accomplished using fermentation techniques. One method for maximizing production of fatty acid derivatives while reducing costs is increasing the percentage of the carbon source that is converted to hydrocarbon products.

During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth).

The host cell can be additionally engineered to express recombinant cellulosomes, such as those described in Published U.S. Patent Application No. 20110097769. These cellulosomes can allow the host cell to use cellulosic material as a carbon source. For example, the host cell can be additionally engineered to express invertases (EC 3.2.1.26) so that sucrose can be used as a carbon source. Similarly, the host cell can be engineered using the teachings described in U.S. Pat. Nos. 5,000,000; 5,028,539; 5,424,202; 5,482,846; and 5,602,030; so that the host cell can assimilate carbon efficiently and use cellulosic materials as carbon sources.

For small scale production, the engineered host cells can be grown in batches of, for example, about 100 mL, 500 mL, 1 L, 2 L, 5 L, or 10 L; fermented; and induced to express desired fatty acid derivative biosynthetic genes based on the specific genes encoded in the appropriate plasmids or incorporated into the host cell's genome. For large scale production, the engineered host cells can be grown in batches of about 10 L, 100 L, 1000 L, 10,000 L, 100,000 L, 1,000,000 L or larger; fermented; and induced to express desired fatty acid derivative biosynthetic genes based on the specific genes encoded in the appropriate plasmids or incorporated into the host cell's genome.

The fatty acid derivatives produced during fermentation can be separated from the fermentation media. Any known technique for separating fatty acid derivatives from aqueous media can be used. One exemplary separation process is a two-phase (bi-phasic) separation process. This process involves fermenting the genetically engineered host cells under conditions sufficient to produce fatty acid derivatives (e.g., fatty alcohols), allowing the fatty acid derivatives to collect in an organic phase, and separating the organic phase from the aqueous fermentation broth. This method can be practiced in both a batch and continuous fermentation processes.

### Advantages and Improvements Provided by the Recombinant Host Cells, Cultures, and Methods of the Present Invention

One facet of the present invention relates to modification of the activity of a β-hydroxyacyl-ACP dehydratase/isomerase protein, having an Enzyme Commission number of EC 4.2.1.60, (e.g., E. coli fabA protein) as a way to modulate aliphatic chain length of fatty acid derivatives produced by a recombinant host cell. This was unexpected because, prior to the present disclosure, the β-hydroxyacyl-ACP dehydratase/isomerase proteins were not believed to be involved in elongation of the aliphatic chains of fatty acid derivatives.

Another facet of the present invention relates to modification of the activity of a β-hydroxyacyl-ACP dehydratase protein that lacks isomerase activity, the protein having an Enzyme Commission number of EC 4.2.1.-, (e.g., E. coli fabZ protein) provides a way to modulate aliphatic chain length of fatty acid derivatives produced by a recombinant host cell. Further, modification of the activity of a β-hydroxyacyl-ACP dehydratase protein that lacks isomerase activity was demonstrated by experiments performed in support of the present invention to provide a way to modulate saturation of aliphatic chains of fatty acid derivatives produced by a recombinant host cell. These discoveries were unexpected because, prior to the present disclosure, (i) the β-hydroxyacyl-ACP dehydratase proteins that lack isomerase activity were not believed to be involved in elongation of the aliphatic chains of fatty acid derivatives; and (ii) these proteins lack isomerase activity and thus they were not believed to affect saturation.

Yet another facet of the present invention relates to the discovery that balancing of the activities of (i) proteins involved in the elongation of the aliphatic chains of fatty acid derivatives (e.g., elongation β-ketoacyl-ACP synthase proteins, having an Enzyme Commission number of EC 2.3.1.-; such as, E. coli fabB protein and E. coli fabF protein), and (ii) proteins involved in the termination of fatty acid derivative synthesis (e.g., thioesterases, having an Enzyme Commission number of EC 3.1.1.5 or EC 3.1.2.-; such as, an E. coli tesA thioesterase protein), in recombinant host cells provides a way to produce high titers of fatty acid derivatives having targeted aliphatic chain lengths. This facet of the present invention provides the means to make and use recombinant host cells to produce high titers of fatty acid derivatives having targeted aliphatic chain lengths, which is an important advancement in the field of producing fatty acid derivatives from renewable resources to reduce reliance on petrochemical sources.

### EXAMPLES

The following examples are put forth so as to provide those of ordinary skill in the art with a complete disclosure and description of how to practice the present invention, and are not intended to limit the scope of what the inventors regard as the invention. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric.

### Example 1

### Examples of Expression Constructs

FIG. 3 presents various genetic constructions used to illustrate the recombinant microorganisms, cultures, and methods of certain embodiments of the present invention. The genes designated in the figure can be found in Table 1. The genes comprised regulatory regions (R) operably-linked to polynucleotide sequence encoding the protein products. R2 through R6 were different regulatory elements comprising ribosome binding:sites and translational termination signals.

The base plasmid OP-80 was generated from the commercially available plasmid pCL1920 (Lerner et al., Nucleic Acids Res. 18: 4631 (1990)). The pCL1920 plasmid was modified to comprise the P_{TRC} promoter and the lacI sequences, which were obtained from the plasmid pTrcHis2 (Invitrogen Corporation, Carlsbad, Calif.). The constructions, schematically illustrated in FIG. 3, were incorporated into the OP-80 base plasmid adjacent and operably-linked to the Ptrc promoter.

### Example 2

### Examples of Bacterial Strains

Table 2 presents the genetic characterization of a number of E. coli K12 strains into which plasmids containing the expression constructs of FIG. 3 (Example 1) were introduced as described below. These strains and plasmids were used to demonstrate the recombinant microorganisms, cultures, and methods of certain embodiments of the present invention. The genetic designations in Table 2 are standard designations known to those of ordinary skill in the art.

TABLE 2

| Strain | E. coli | |
|---|---|---|
| Name | type | Genetic Characterization |
| DV2 | K12 | F-, λ-, ilvG-, rfb-50, rph-1, ΔfhuA::FRT, ΔfadE::FRT |
| D178 | K12 | F-, λ-, ilvG-, rfb-50, rph-1, ΔfhuA::FRT, ΔfadE::FRT, |
| | | fabB[A329V]::FRT, P_{T5} entD |
| EG149 | K12 | F-, λ-, ilvG-, rfb-50, rph-1, ΔfhuA::FRT, ΔfadE::FRT, |
| | | fabB[A329V]::FRT, P_{T5}_entD, insH-11::(P_{LACUV5}- |
| | | V_{cho}_fabV-S_{typ}_(fabHDG)-S_{typ}_fabA-C_{ace}_fabF::FRT) |
| V668 | K12 | F-, λ-, ilvG⁺, rfb-50, rph⁺, ΔfhuA::FRT, ΔfadE::FRT, |
| | | fabB[A329V]::FRT, P_{T5}_entD, insH-11::(P_{LACUV5}- |
| | | V_{cho}_fabV-S_{typ}_(fabHDG)-S_{typ}_fabA-C_{ace}_fabF::FRT) |

### Example 3

### Optimizing Production and Aliphatic Chain Lengths of Fatty Acid Derivatives

The data in this example provide a clear illustration of the usefulness of embodiments of the methods of the present invention to make recombinant host cells engineered to produce high titers of fatty acid derivatives having targeted aliphatic chain lengths. The example sets forth results of the methods described herein to optimize fatty acid derivative production by optimizing the expression/activities of both an elongation β-ketoacyl-ACP synthase protein (here the E. coli fabB protein) and a thioesterase (here the E. coli tesA protein).

### A. Optimizing Titer of Fatty Acid Derivatives

The following data provide an example of method step (B) as described herein. Experiments performed in support of the present invention demonstrated that manipulation of the expression of thioesterase (here, the E. coli tesA, thioesterase protein) can facilitate optimal production of fatty acid derivatives.

TesA expression was optimized by modulating the activity of the 5′ non-coding polynucleotide sequence (comprising operably-linked regulatory sequences) adjacent the 5′-end of the open reading frame of the tesA gene (FIG. 3, panel A, R2) via randomization of the regulatory sequences. Region R2, the regulatory sequences operably-linked to the thioesterase coding sequence, were modified by randomization of the non-coding polynucleotide sequences to create a plasmid library. The plasmid library comprised the randomized expression construct illustrated in FIG. 3, panel A, carried in the base plasmid OP-80. This library was transformed into a cloning strain (TOP10; Invitrogen Corporation, Carlsbad, Calif.) and colonies selected using Luria-Bertani agar plates containing an appropriate antibiotic. Surviving colonies were pooled and the DNA was extracted using standard protocols to provide the library.

The resulting library was transformed into strain DV2 (Example 2).to prepare a group of recombinant microorganisms for screening. Spectinomycin (100 μg/mL) was included in all media to maintain selection of the exogenous, plasmid DNA.

Briefly, colonies (clones) were picked and inoculated into glass culture tubes containing 2 mL of Luria-Bertani (LB) medium. After overnight growth, 50 μL of each tube was transferred to a new tube of fresh LB medium. The clones were cultured for 3 hours after which each culture was used to inoculate 20 mL of V-9 media in a 125 mL flask. V-9 medium is M9 medium with 2% glucose supplemented with antibiotics, 1 μg/L thiamine, and a 1:1000 dilution of the trace mineral solution described in Table 3.

TABLE 3
Trace mineral solution
(filter sterilized)

| | |
|---|---|
| 2 g/L | ZnCl•4H₂O |
| 2 g/L | CaCl₂•6H₂O |
| 2 g/L | Na₂MoO₄•2H₂O |
| 1.9 g/L | CuSO₄•5H₂O |
| 0.5 g/L | H₃BO₃ |
| 100 mL/L | concentrated HCl |

q.s. Milli-Q water

At an OD600 of 1.0, 1 mM IPTG was added to the culture to induce protein expression. After 20 hours of fermentation, the cultures were extracted with butyl acetate in preparation for screening. The crude extracts were derivatized with BSTFA (N,O-bis[Trimethylsilyl]trifluoroacetamide) and the titer of fatty alcohols and, free fatty acids (combined) were measured with GC-FID as described in U.S. Patent Publication No. 20100251601, published 7 Oct. 2010.

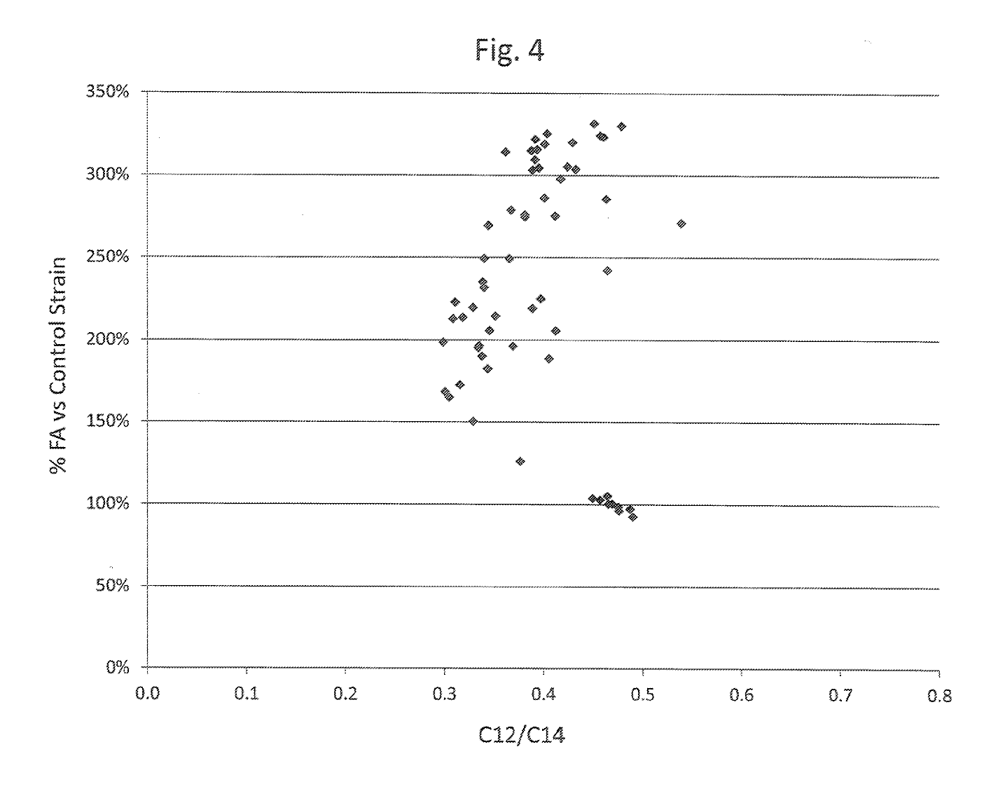

The data in the figure demonstrate that the method provided high titer clones with more than a 3-fold increase in the titer of fatty derivatives produced by the engineered recombinant microorganisms (e.g., FIG. 4, data points above the 300% line) relative to the control microorganisms.

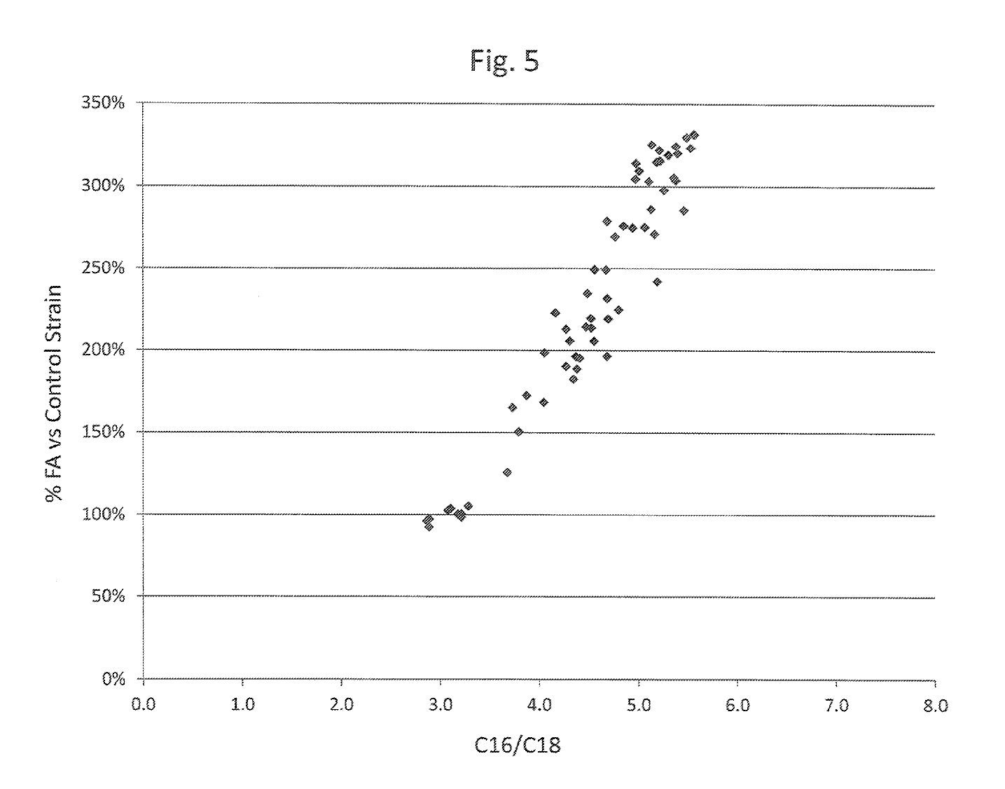

FIG. 5 presents screening data for clones wherein the activity of the thioesterase protein in the recombinant microorganisms was modified relative to the thioesterase protein activity in the control microorganism. In the figure, the Y-axis is “% FA vs. Control Strain,” as described for FIG. 4. The X-axis is the C₁₆/C₁₈ ratio for titers of fatty acid derivatives (combined free fatty acids and fatty alcohols) having C₁₆ and C₁₈ aliphatic chain lengths. The data points in the figure each correspond to a cultured clone or the control strain. In the figure, the four data points clustered near 100% correspond to cultures of the control strain.

The data in the figure demonstrate that the method provided high titer clones with more than a 3-fold increase in the titer of fatty derivatives produced by the engineered recombinant microorganisms (e.g., FIG. 5, data points above the 300% line) relative to the control microorganisms.

These data demonstrated that using the methods of the present invention recombinant microorganisms were obtained that provided significant increase in titer relative to control microorganisms. Further, in view of the ranges of the C_{X}/C_{Y}, culturing engineered recombinant microorganisms of the present invention provide a range of tailored, target aliphatic chain lengths of fatty acid derivatives.

The engineered recombinant microorganism that produced the maximum titer was selected for use in the following method.

### B. Optimizing Titer and Aliphatic Chain Lengths of Fatty Acid Derivatives

The following data provide an example of method step (A) as described herein. Experiments performed in support of the present invention demonstrated that manipulation of the expression of the elongation β-ketoacyl-ACP synthase protein (here, the E. coli fabB, 3-oxoacyl-[acyl-carrier-protein] synthase I protein) can facilitate optimal production of fatty acid derivatives having target aliphatic chain lengths.

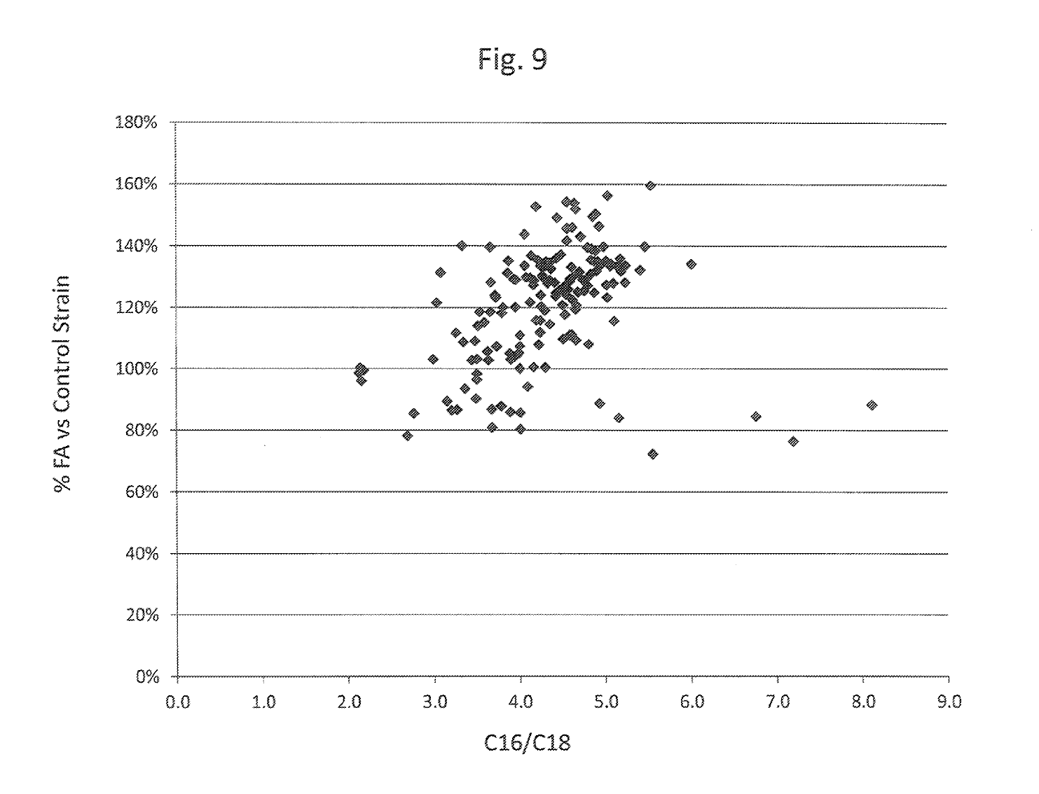

Plasmid DNA from the highest producer from the above-described library was purified and the polynucleotide comprising the R2-tesA gene was isolated. The tesA protein coding sequence was replaced with a nucleotide sequence encoding the tesA(13G04) protein (FIG. 5C; SEQ ID NO:17). The R2-tesA(13G04) was incorporated into the construct illustrated in FIG. 9, panel B (i.e., the starting polynucleotide). Thus, the following data also provide an example of method step (B) followed by method step (A).

FabB expression was optimized by modulating the activity of the 5′ non-coding polynucleotide sequence (comprising operably-linked regulatory sequences) adjacent the 5′-end of the open reading frame of the fabB gene (FIG. 9, panel B, R4) via randomization of the regulatory sequences. Region R4, the regulatory sequences operably-linked to the 3-oxoacyl-[acyl-carrier-protein] synthase I protein coding sequence, were modified by randomization of the non-coding polynucleotide sequences to create a plasmid library. The plasmid library comprised the randomized expression construct illustrated in FIG. 9, panel B, carried in the base plasmid OP-80; wherein the R2 associated with the tesA(13G04) coding sequence of the construct was the R2 isolated from the highest producer described above. This library was transformed into a cloning strain (e.g., TOP10; Invitrogen Corporation, Carlsbad, Calif.) and colonies selected using Luria-Bertani agar plates containing an appropriate antibiotic. Surviving colonies were pooled and the DNA was extracted using standard protocols to provide the library of the E. coli fabB gene.

The resulting library was transformed into strain D178 (Example 2, Table 2) to prepare a group of recombinant microorganisms for screening. Spectinomycin (100 μg/mL) was included in all media to maintain selection of the exogenous, plasmid DNA. Briefly, colonies (clones) were picked and used to inoculate wells of 96 well plates containing Luria-Bertani (LB) medium. After overnight growth, 40 μL was transferred from each well in the plate to a new well in a new plate with fresh LB. After 3 hours growth, 40 μL of each culture was used to inoculate 400 μL of FA2 media in 96 well plates. FA2 medium is M9 medium with 3% glucose supplemented with antibiotics, 1 μg/L thiamine, 10 μg/L iron citrate, and a 1:1000 dilution of the trace mineral solution described in Table 3.

After 5 hours of growth, at an OD600 of 1.0, 1 mM IPTG was added to the culture to induce protein expression. After 20 hours of fermentation, the cultures were extracted with butyl acetate in preparation for screening. The crude extracts were derivatized with BSTFA (N,O-bis[Trimethylsilyl]trifluoroacetamide) and the titer of fatty alcohols and free fatty acids (combined) were measured with GC-FID as described in U.S. Patent Publication No. 20100251601, published 7 Oct. 2010.

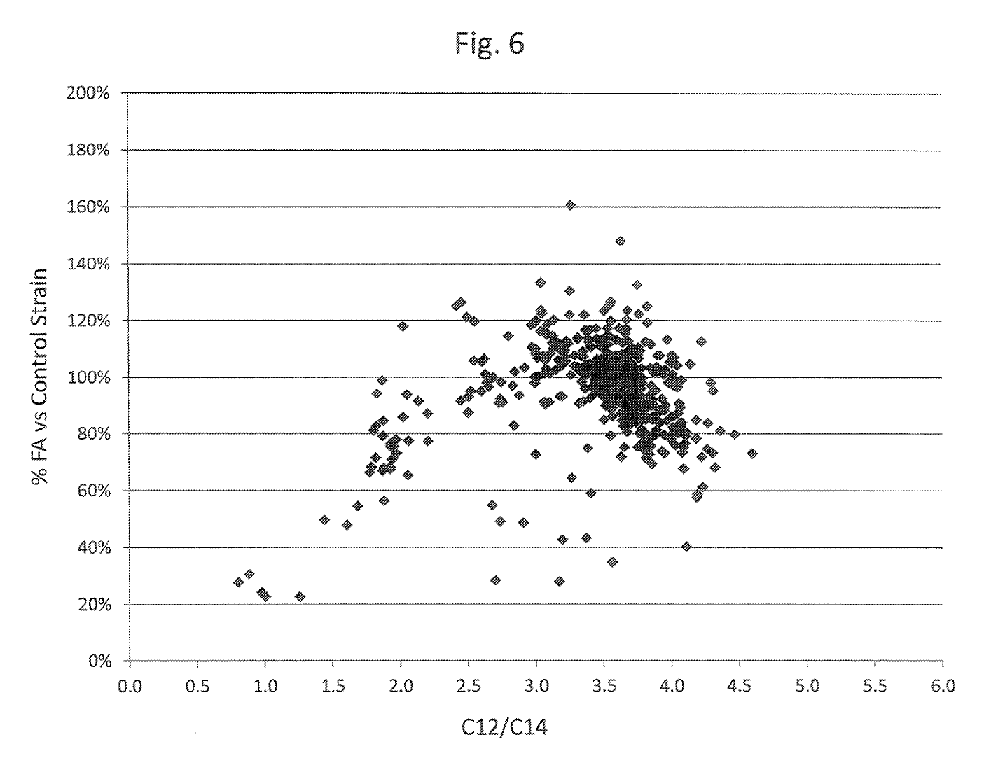

FIG. 6 presents screening data for clones wherein the activity of the elongation β-ketoacyl-ACP synthase protein (here, the E. coli fabB, 3-oxoacyl-[acyl-carrier-protein] synthase I protein) in the recombinant microorganisms was modified relative to the elongation β-ketoacyl-ACP synthase protein activity in the control microorganism (here, the E. coli fabB, 3-oxoacyl-[acyl-carrier-protein] synthase I protein). In the figure, the Y-axis is “% FA vs. Control Strain,” the % FA being the total measured titer of fatty acid derivatives (here the combined free fatty acids and fatty alcohols) including all aliphatic chain lengths for each clone divided by the total measured titer of fatty acid derivatives (here the combined free fatty acids and fatty alcohols) including all aliphatic-chain lengths for the “Control Strain.” Here the “Control Strain” was an E. coli strain that had been previously engineered to produce a good titer of fatty acid derivatives; thus the 100% line indicates clones that produced comparable titer to the “Control Strain.” The X-axis is the C₁₂/C₁₄ ratio for titers of fatty acid derivatives (combined free fatty acids and fatty alcohols) having C₁₂ and C₁₄ aliphatic chain lengths. The data points in the figure each correspond to a cultured clone or a “Control Strain.” Four of the data points clustered near 100% correspond to cultures of the “Control Strain” which were used as controls and points for comparison.

The data in the figure demonstrate that the method provided high titer clones of engineered recombinant microorganisms with a significant increase in the titer of a fatty acid derivative having a target aliphatic chain length (e.g., FIG. 6, a fatty acid derivative having a target aliphatic chain length characterized by a C₁₂/C₁₄ ratio of about 3.1 with a titer of, 160%; thus an improvement of 1.5-fold) compared to the “Control Strain.”

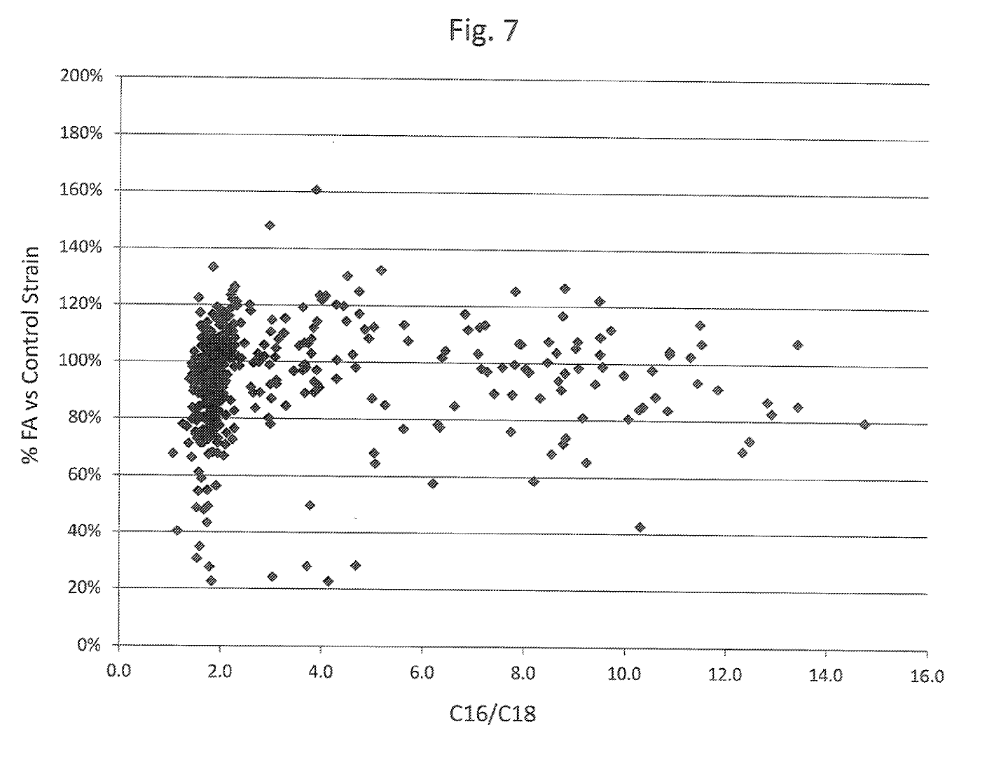

FIG. 7 presents screening data for clones wherein the activity of the elongation β-ketoacyl-ACP synthase protein (here, the E. coli fabB, 3-oxoacyl-[acyl-carrier-protein] synthase I protein) in the recombinant microorganisms was modified relative to the elongation β-ketoacyl-ACP synthase protein activity in the control microorganism (here, the E. coli fabB, 3-oxoacyl-[acyl-carrier-protein] synthase I protein). In the figure, the Y-axis is “% FA vs. Control Strain,” as described for FIG. 6. The X-axis is the C₁₆/C₁₈ ratio for titers of fatty acid derivatives (combined free fatty acids and fatty alcohols) having C₁₆ and C₁₈ aliphatic chain lengths. The data points in the figure each correspond to a cultured clone or a “Control Strain.” Four of the data points clustered near 100% correspond to cultures of the “Control Strain” which were used as controls and points for comparison.

The data in the figure demonstrate that the method provided high titer clones of engineered recombinant microorganisms with a significant increase in the titer of a fatty acid derivative having a target aliphatic chain length (e.g., FIG. 7, a fatty acid derivative having a target aliphatic chain length characterized by a C₁₆/C₁₈ ratio of about 4.0 with a titer of 160%, thus an improvement of 1.5-fold) compared to the “Control Strain.”

These data demonstrated that using the methods of the present invention, recombinant microorganisms were obtained that provided significant increase in titer for fatty acid derivatives having different aliphatic chain lengths, thus showing the flexibility of the method to provide fatty acid derivatives having any of a multitude of target aliphatic chain lengths.

### C. Further Optimization of Titer and Aliphatic Chain Lengths of Fatty Acid Derivatives

The following data provide another example of method step (B) as described, herein. Experiments performed in support of the present invention demonstrated that manipulation of the expression of thioesterase (here, the E. coli tesA, thioesterase protein) can facilitate optimal production of fatty acid derivatives. Repeating step (B) using a recombinant microorganism selected, for example, from a previous step (A) provides a way to isolate further recombinant microorganisms having increased productivity of fatty acid derivatives relative to the productivity of the recombinant microorganism from the previous step (A).

Two different clones from the fabB library of Example 3B were used to generate a new tesA library. Neither of these strains were the highest producer in the library, that is, the strains had titers less than maximum titer of the group of recombinant microorganism from which they were selected. Further, the two clones were selected from those producing longer aliphatic chain lengths, as measured by both the ratio of C₁₂/C₁₄ and C₁₆/C₁₈. For example, with reference to FIG. 6 and FIG. 7, the two clones had titers less than the maximum titer (FIG. 6 and FIG. 7, the data point at 160% is clearly the maximum titer). Each of the two clones had a C_{X}/C_{Y} ratio less than an example target aliphatic chain length C_{X}/C_{Y} ratio as follows: for C₁₂/C₁₄ an example target ratio of C₁₂/Q₁₄˜3.2 (FIG. 6, the data point at 3.1 on the X-axis and 160% on the Y-axis), the two clones were selected that had titers of less than 160% and C₁₂/C₁₄ ratios of less than ˜3.1; and, for C₁₆/C₁₈ an example target ratio of C₁₆/C₁₅˜4.0 (FIG. 7, the data point at 4.0 on the X-axis and 160% on the Y-axis), the two clones were selected that had titers of less than 160% and C₁₆/C₁₈ ratios of less than ˜4.0.

Plasmid DNA was isolated from each of the two clones from the fabB library of Example 3B, and the plasmid DNAs were used to construct the starting polynucleotides (FIG. 9, panel B, R4). The starting polynucleotides were used for the generation of a new tesA library. Thus, the following data also provide an example of method step (B) followed by method step (A) followed by method step (B).

TesA expression was optimized by modulating the activity of the 5′ non-coding polynucleotide sequence (comprising operably-linked regulatory sequences) adjacent the 5′-end of the open reading frame of the tesA gene (FIG. 9, panel B, R2) via randomization of the regulatory region. The tesA protein coding sequence was a polynucleotide sequence encoding the tesA(12H08) protein (FIG. 5D; SEQ ID NO:19). Region R2, the regulatory sequences operably-linked to the thioesterase coding sequence, were modified by randomization of the non-coding polynucleotide sequences to create a plasmid library. The plasmid library comprised the randomized expression construct illustrated in FIG. 9, panel B, carried in the base plasmid OP-80. This library was transformed into a cloning strain (TOP 10; Invitrogen Corporation, Carlsbad, Calif.) and colonies selected using Luria-Bertani agar plates containing an appropriate antibiotic. Surviving colonies were pooled and the DNA was extracted using standard protocols to provide the library.

The resulting library was transformed into strain EG149 (Example 2, Table 2) to prepare a group of recombinant microorganisms for screening. Spectinomycin (100 μg/mL) was included in all media to maintain selection of the exogenous, plasmid DNA. Briefly, colonies (clones) were picked and used to inoculate 96 well plates containing Luria-Bertani (LB) medium. After overnight growth, 40 μL was transferred from each well in the plate to a new well in a new plate with fresh LB. After 3 hours growth, 40 μL of each culture was used to inoculate 400 μL of FA2 media in 96 well plates.

After 5 hours of growth, at an OD600 of 1.0, 1 mM IPTG was added to the culture to induce protein expression. After 20 hours of fermentation, the cultures were extracted with butyl acetate in preparation for screening. The crude extracts were derivatized with BSTFA (N,β-bis[Trimethylsilyl]trifluoroacetamide) and the titer of fatty alcohols and free fatty acids (combined) were measured with GC-FID as described in U.S. Patent Publication No. 20100251601, published 7 Oct. 2010.

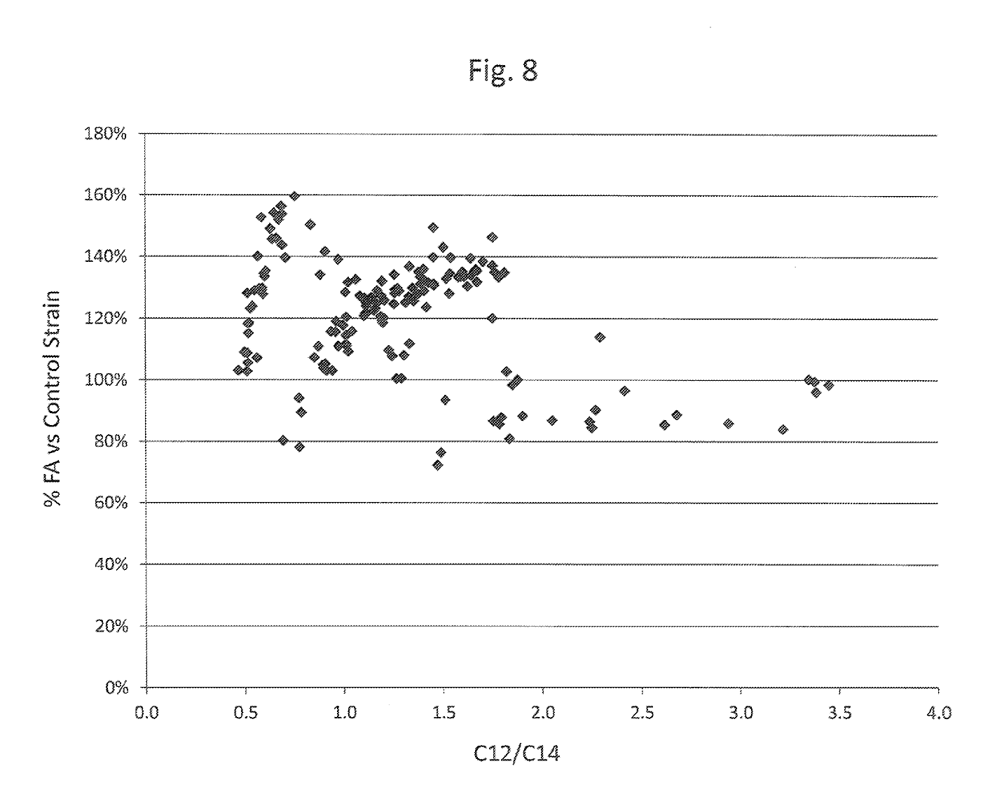

FIG. 8 presents screening data for clones wherein the activity of the thioesterase protein in the recombinant microorganisms was modified relative to the thioesterase protein activity in the control microorganism. In the figure, the Y-axis is “% FA vs. Control Strain,” as described for FIG. 6. The X-axis is the C₁₂/C₁₄ ratio for titers of fatty acid derivatives (combined free fatty acids and fatty alcohols) having C₁₂ and C₁₄ aliphatic chain lengths. The data points in the figure each correspond to a cultured clone or a “Control Strain.”

The data in the figure demonstrate that the method provided high titer clones of engineered recombinant microorganisms with a significant increase in the titer of a fatty acid derivative having a target aliphatic chain length (e.g., FIG. 8, using an exemplary target aliphatic chain length characterized by a C₁₂/C₁₄ ratio of between about 1.5 and about 2.0) compared to the “Control Strain.”

FIG. 9 presents screening data for clones wherein the activity of the thioesterase protein in the recombinant microorganisms was modified relative to the thioesterase protein activity in the control microorganism. In the figure, the Y-axis is “% FA vs. Control Strain,” as described for FIG. 6. The X-axis is the C₁₆/C₁₈ ratio for titers of fatty acid derivatives (combined free fatty acids and fatty alcohols) having C₁₆ and C₁₈ aliphatic chain lengths. The data points in the figure each correspond to a cultured clone or a “Control Strain.” The data in the figure demonstrate that the method provided high titer clones of engineered recombinant microorganisms with a significant increase in the titer of a fatty acid derivative having a target aliphatic chain length (e.g., FIG. 9, using an exemplary target aliphatic chain length characterized by a C₁₆/C₁₈ ratio of between ˜4.0 and ˜5.0) compared to the “Control Strain.”

These data demonstrated that using the methods of the present invention, recombinant microorganisms were obtained that provided significant increase in titer for a multitude of different aliphatic chain lengths, thus showing the flexibility of the method to provide fatty acid derivatives having any of a multitude of target aliphatic chain lengths.

### Example 4

### Optimizing Saturation of the Aliphatic Chains of Fatty Acid Derivatives

The data in this example provide a clear illustration of the usefulness of embodiments of the methods of the present invention to make recombinant host cells engineered to produce fatty acid derivatives having targeted aliphatic chain lengths with desired levels of saturation. The example sets forth results of the methods described herein to optimize fatty acid derivative production by optimizing the expression/activities of both an elongation β-ketoacyl-ACP synthase protein (here 3-oxoacyl-[acyl-carrier-protein] synthase protein, the E. coli fabB protein) and β-hydroxyacyl-ACP dehydratase protein (here β-hydroxydecanoyl thioester dehydratase/isomerase protein, the E. coli FabA protein, and (3R)-hydroxymyristol acyl carrier protein dehydratase protein, the E. coli FabZ protein).

A. The E. coli fabB Protein

Both saturation and chain length of fatty acid derivatives can be optimized using the E. coli fabB gene encoding 3-oxoacyl-[acyl-carrier-protein] synthase I protein.

Plasmid DNA from the highest producer from the above-described library in Example 3A was purified and the polynucleotide comprising the R2-tesA gene was isolated. The tesA protein coding sequence was replaced with a nucleotide sequence encoding the tesA(13G04) protein (FIG. 5C; SEQ ID NO:17). Thus, the following data also provide an example of method step (B) followed by method step (C) using one or more polynucleotide sequence including an open reading frame encoding an elongation β-ketoacyl-ACP synthase protein as an alternative to one or more polynucleotide sequences including an open reading frame encoding a β-hydroxyacyl-ACP dehydratase protein.

FabB expression was modulated by randomizing the 5′ non-coding polynucleotide sequence (comprising operably-linked regulatory sequences) adjacent the 5′-end of the open reading frame of the fabB gene (FIG. 9, panel B, R4). Region R4, the regulatory sequences operably-linked to the 3-oxoacyl-[acyl-carrier-protein] synthase I protein coding sequence, were modified by randomization of the non-coding polynucleotide sequences to create a plasmid library. The plasmid library comprised the mutagenized expression construct illustrated in FIG. 9, panel B, carried in the base plasmid OP-80; wherein the R2-tesA gene of the construct was the R2-tesA(13G04) gene isolated as described above. This library was transformed into a cloning strain (TOP10; Invitrogen Corporation, Carlsbad, Calif.) and colonies selected using Luria-Bertani agar plates containing an appropriate antibiotic. Surviving colonies were pooled and the DNA was extracted using standard protocols to provide the library of the E. coli fabB gene.

The resulting library was transformed into strain D178 (Example 2, Table 2) to prepare a group of recombinant microorganisms for screening. Spectinomycin (100 μg/mL) was included in all media to maintain selection of the exogenous, plasmid DNA. Briefly, colonies (clones) were picked and used to inoculate wells of 96 well plates containing Luria-Bertani (LB) medium. After overnight growth, 40 μL was transferred from each well in the plate to a new well in a new plate with fresh LB. After 3 hours growth, 40 μL of each culture was used to inoculate 400 μL of FA2 media in 96 well plates.

After 5 hours of growth, at an OD600 of 1.0, 1 mM IPTG was added to the culture to induce protein expression. After 20 hours of fermentation, the cultures were extracted with butyl acetate in preparation for screening. The crude extracts were derivatized with BSTFA (N,β-bis[Trimethylsilyl]trifluoroacetamide) and the titer of fatty alcohols and free fatty acids (combined) were measured with GC-FID as described in U.S. Patent Publication No. 20100251601, published 7 Oct. 2010.

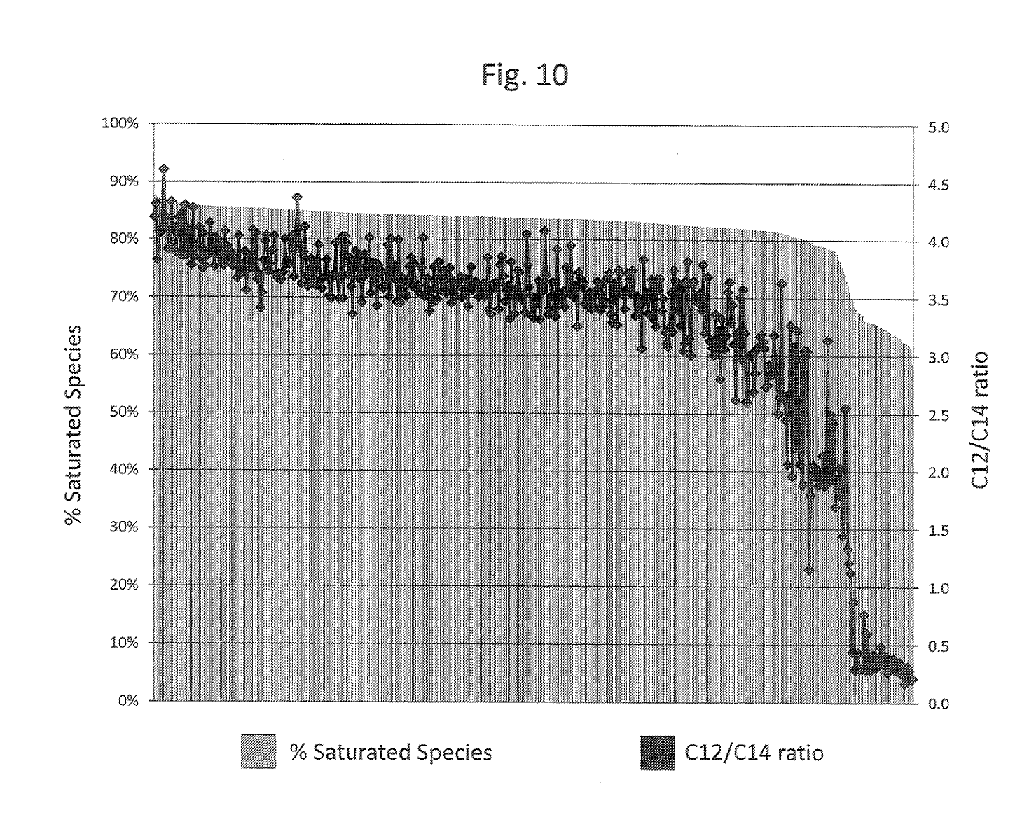

FIG. 10 presents screening data for clones wherein the activity of the elongation β-ketoacyl-ACP synthase protein (here, the E. coli fabB, 3-oxoacyl-[acyl-carrier-protein] synthase I protein) in the recombinant microorganisms was modified relative to the elongation β-ketoacyl-ACP synthase protein activity in the control microorganism (here, the E. coli fabB, 3-oxoacyl-[acyl-carrier-protein] synthase I protein). In the figure, the left Y-axis is “% Saturated Species,” which is the measured titer of fatty acid derivatives (here the combined free fatty acids and fatty alcohols) having saturated aliphatic chains and including all aliphatic chain lengths for each clone divided by the total measured titer of fatty acid derivatives (here the combined free fatty acids and fatty alcohols) including all aliphatic chain lengths. The right Y-axis is the C₁₂/C₁₄ ratio for titers of fatty acid derivatives (combined free fatty acids and fatty alcohols) having C₁₂ and C₁₄ aliphatic chain lengths. The data points in the figure each correspond to a cultured clone or a control. Four of the data points correspond to cultures of the “Control Strain” (as in FIG. 6, described above) that were used as controls and points for comparison. The clones from the screened group of recombinant microorganisms are arranged along the X-axis based on their % Saturated Species and the corresponding data points for their C₁₂/C₁₄ ratios are shown.

Analyses of the data in the figure demonstrate that the methods of the present invention provide engineered, recombinant microorganisms that produce a wide range of aliphatic chain lengths of fatty acid derivatives, from which one of ordinary skill in the art can select desired target aliphatic chain lengths, with desired levels of saturation. The methods, recombinant microorganisms and cultures of the present invention give one of ordinary skill in the art the tools to tailor aliphatic chain lengths and saturation to achieve desired results.

B. The E. coli fabA Protein

Both saturation and chain lengths of fatty acid derivatives can be optimized using the E. coli fabA gene encoding β-hydroxydecanoyl thioester dehydratase/isomerase protein.

Plasmid DNA from the above-described library in Example 3C was purified and the polynucleotide comprising the R2-tesA(12H08) gene and R4-fabB gene was isolated. Thus, the following data also provide an example of method step (B) followed by method step (A) followed by method step (B) followed by method step (C).

FabA expression was modulated by randomization of the 5′ non-coding polynucleotide sequence (comprising operably-linked regulatory sequences) adjacent the 5′-end of the open reading frame of the fabA gene (FIG. 9, panel C, R6). Region R6, the regulatory sequences operably-linked to the β-hydroxydecanoyl thioester dehydratase/isomerase protein coding sequence, were modified by randomization of the non-coding polynucleotide sequences to create a plasmid library. The plasmid library comprised the randomized expression construct illustrated in FIG. 9, panel C, carried in the base plasmid OP-80; wherein the R2-tesA and R4-fabB gene of the construct were the R2-tesA(12H08) gene and R4-fabB gene obtained in Example 3C. This library was transformed into a cloning strain (TOP 10; Invitrogen Corporation, Carlsbad, Calif.) and colonies selected using Luria-Bertani agar plates containing an appropriate antibiotic. Surviving colonies were pooled and the DNA was extracted using standard protocols to provide the library.

The resulting library was transformed into strain V668 (Example 2, Table 2) to prepare a group of recombinant microorganisms for screening. Spectinomycin (100 μg/mL) was included in all media to maintain selection of the exogenous, plasmid DNA. Briefly, colonies (clones) were picked and used to inoculate wells of 96 well plates containing Luria-Bertani (LB) medium. After overnight growth, 40 μL was transferred from each well in the plate to a new well in a new plate with fresh LB. After 3 hours growth, 40 μL of each culture was used to inoculate 400 μL of FA2 media in 96 well plates.

After 5 hours of growth, at an OD600 of 1.0, 1 mM 1PTG was added to the culture to induce protein expression. After 20 hours of fermentation, the cultures were extracted with butyl acetate in preparation for screening. The crude extracts were derivatized with BSTFA (N,O-bis[Trimethylsilyl]trifluoroacetamide) and the titer of fatty alcohols and free fatty acids (combined) were measured with GC-FID as described in U.S. Patent Publication No. 20100251601, published 7 Oct. 2010.

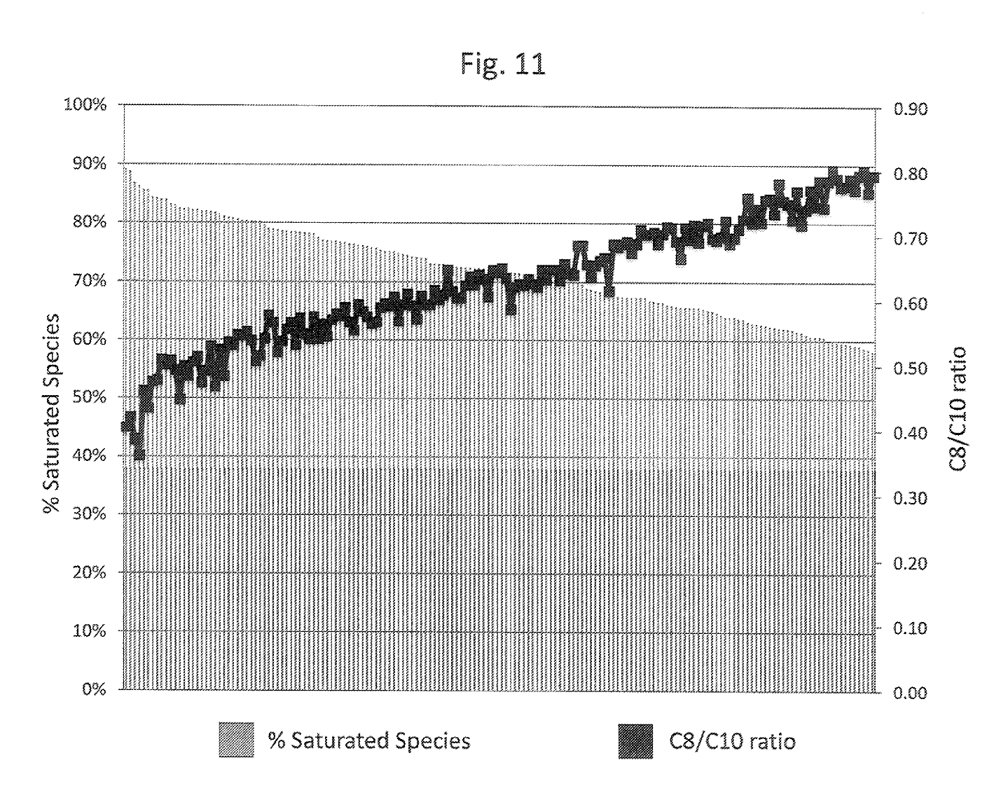

FIG. 11 presents screening data for clones wherein the activity of the β-hydroxyacyl-ACP dehydratase protein (hereβ-hydroxydecanoyl thioester dehydratase/isomerase protein the E. coli FabA protein) in the recombinant microorganisms was modified relative to the β-hydroxyacyl-ACP dehydratase protein (here β-hydroxydecanoyl thioester dehydratase/isomerase protein the E. coli FabA protein) activity in the control microorganism. In the figure, the left Y-axis is “% Saturated Species,” which is the measured titer of fatty acid derivatives (here the combined free fatty acids and fatty alcohols) having saturated aliphatic chains and including all aliphatic chain lengths for each clone divided by the total measured titer of fatty acid derivatives (here the combined free fatty acids and fatty alcohols) including all aliphatic chain lengths. The right Y-axis is the C₈/C₁₀ ratio for titers of fatty acid derivatives (combined free fatty acids and fatty alcohols) having C₈ and C₁₀ aliphatic chain lengths. The data points in the figure each correspond to a cultured clone or a control. The clones from the screened group of recombinant microorganisms are arranged along the X-axis based on their % Saturated Species and the corresponding data points for their C₈/C₁₀ ratios are shown.

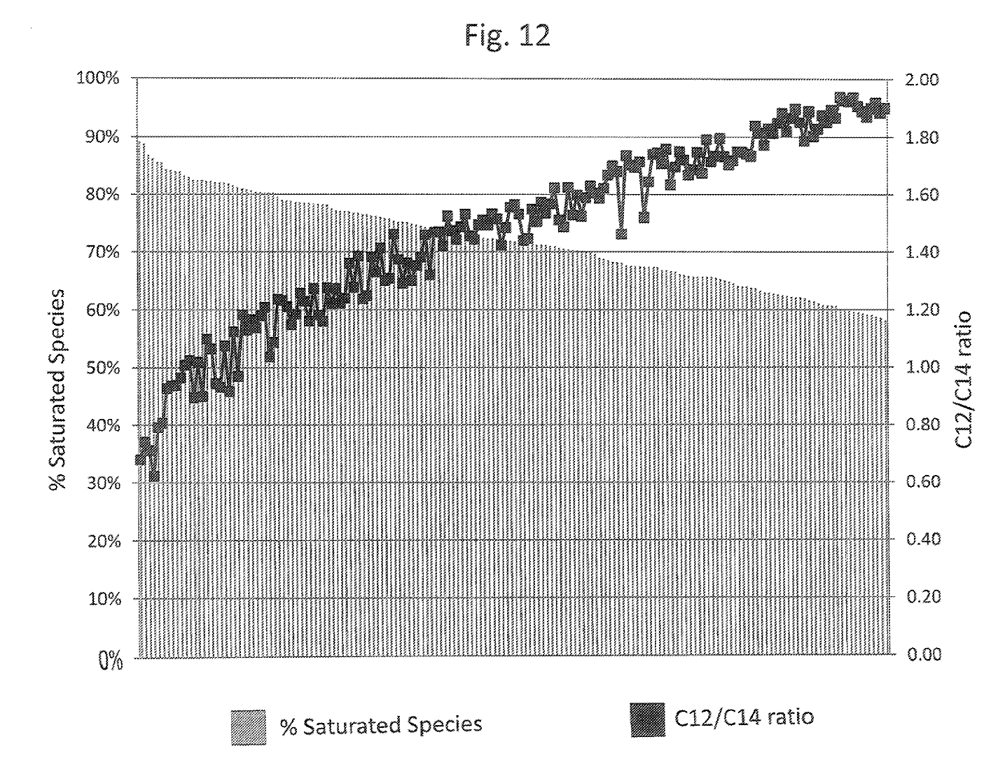

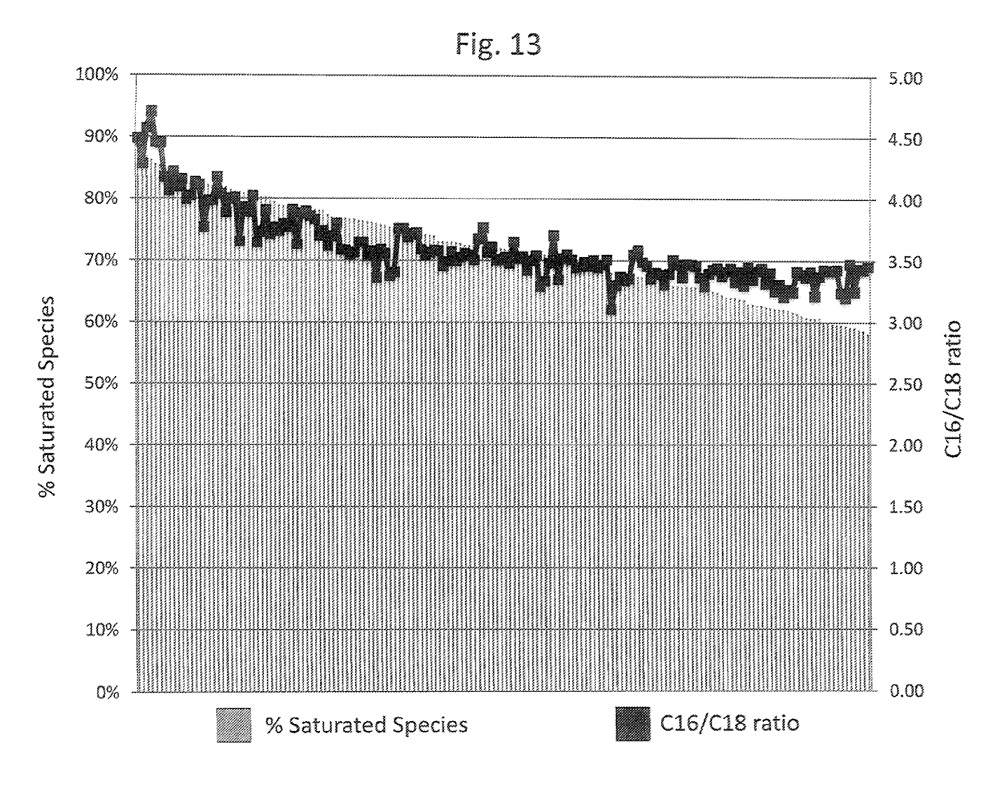

Similar data analyses are shown in FIG. 12 and FIG. 13 for target aliphatic chain lengths characterized by C₁₂/C₁₄ and C₁₆/C₁₈, respectively.

Analyses of the data in the figures demonstrate that the methods of the present invention provide engineered, recombinant microorganisms that produce a wide range of aliphatic chain lengths of fatty acid derivatives, from which one of ordinary skill in the art can select desired target aliphatic chain lengths, with desired levels of saturation. The methods, recombinant microorganisms and cultures of the present invention give one of ordinary skill in the art the tools to tailor aliphatic chain length and saturation to achieve desired results.

C. The E. coli fabZ Protein

Both saturation and chain length of the fatty acid derivatives can be optimized using the E. coli fabZ gene encoding (3R)-hydroxymyristol acyl carrier protein dehydratase protein.

Plasmid DNA from the above-described library in Example 3C was purified and the polynucleotide comprising the R2-tesA(12H08) gene and R4-fabB gene was isolated. Thus, the following data also provide an example of method step (B) followed by method step (A) followed by method step (B) followed by method step (C).

FabZ expression was modulated by randomizing the 5′ non-coding polynucleotide sequence (comprising operably-linked regulatory sequences) adjacent the 5′-end of the open reading frame of the fabZ gene (FIG. 9, panel D, R6). Region R6, the regulatory sequences operably-linked to the (3R)-hydroxymyristol acyl carrier protein dehydratase protein coding sequence, were modified by randomization of the non-coding polynucleotide sequences to create a plasmid library. The plasmid library comprised the randomized expression construct illustrated in FIG. 9, panel D, carried in the base plasmid OP-80; wherein the R2-tesA gene and R4-fabB gene of the construct were the tesA(12H08) gene and R4-fabB gene obtained in Example 3C. The high producer was selected based on a target aliphatic chain length characterized by a C₁₂/C₁₄ ratio of about 1.7 to 1.8; for this target aliphatic chain length the high producer made a titer of about 140% (FIG. 84; Example 3C). This library was transformed into a cloning strain (TOP10; Invitrogen Corporation, Carlsbad, Calif.) and colonies selected using Luria-Bertani agar plates containing an appropriate antibiotic. Surviving colonies were pooled and the DNA was extracted using standard protocols to provide the library.

The resulting library was transformed into strain V668 (Example 2, Table 2) to prepare a group of recombinant microorganisms for screening. Spectinomycin (100 μg/mL) was included in all media to maintain selection of the exogenous, plasmid DNA. Briefly, colonies (clones) were picked and used to inoculate wells of 96 well plates containing Luria-Bertani (LB) medium. After overnight growth, 40 μL was transferred from each well in the plate to a new well in a new plate with fresh LB. After 3 hours growth, 40 μL of each culture was used to inoculate 400 μL of FA2 media in 96 well plates.

After 5 hours of growth, at an OD600 of 1.0, 1 mM IPTG was added to the culture to induce protein expression. After 20 hours of fermentation, the cultures were extracted with butyl acetate in preparation for screening. The crude extracts were derivatized with BSTFA (N,O-bis[Trimethylsilyl]trifluoroacetamide) and the titer of fatty alcohols and free fatty acids (combined) were measured with GC-FID as described in U.S. Patent Publication No. 20100251601, published 7 Oct. 2010.

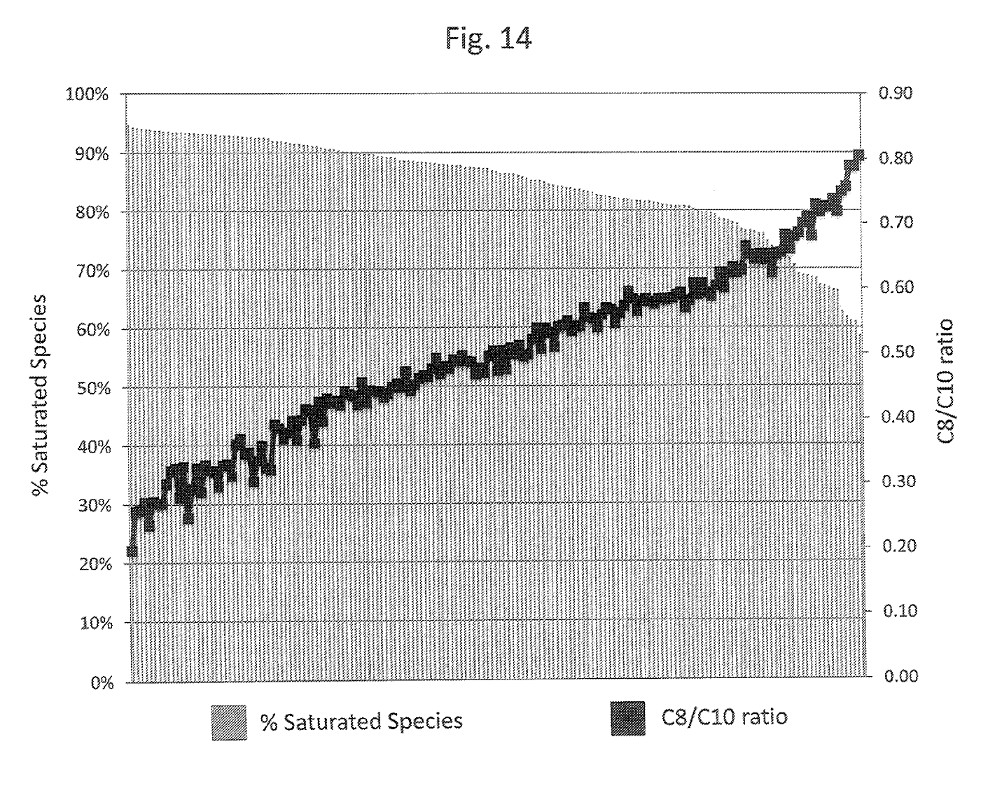

FIG. 14 presents screening data for clones wherein the activity of the β-hydroxyacyl-ACP dehydratase protein (here (3R)-hydroxymyristol acyl carrier protein dehydratase protein, the E. coli FabZ protein) in the recombinant microorganisms was modified relative to theβ-hydroxyacyl-ACP dehydratase protein (here (3R)-hydroxymyristol acyl carrier protein dehydratase protein, the E. coli FabZ protein) activity in the control microorganism. In the figure, the left Y-axis is “% Saturated Species,” which is the measured titer of fatty acid derivatives (here the combined free fatty acids and fatty alcohols) having saturated aliphatic chains and including all aliphatic chain lengths for each clone divided by the total measured titer of fatty acid derivatives (here the combined free fatty acids and fatty alcohols) including all aliphatic chain lengths. The right Y-axis is the C₈/C₁₀ ratio for titers of fatty acid derivatives (combined free fatty acids and fatty alcohols) having C₈ and C₁₀ aliphatic chain lengths. The data points in the figure each correspond to a cultured clone or a control. The clones from the screened group of recombinant microorganisms are arranged along the X-axis based on their % Saturated Species and the corresponding data points for their C₈/C₁₀ ratios are shown.

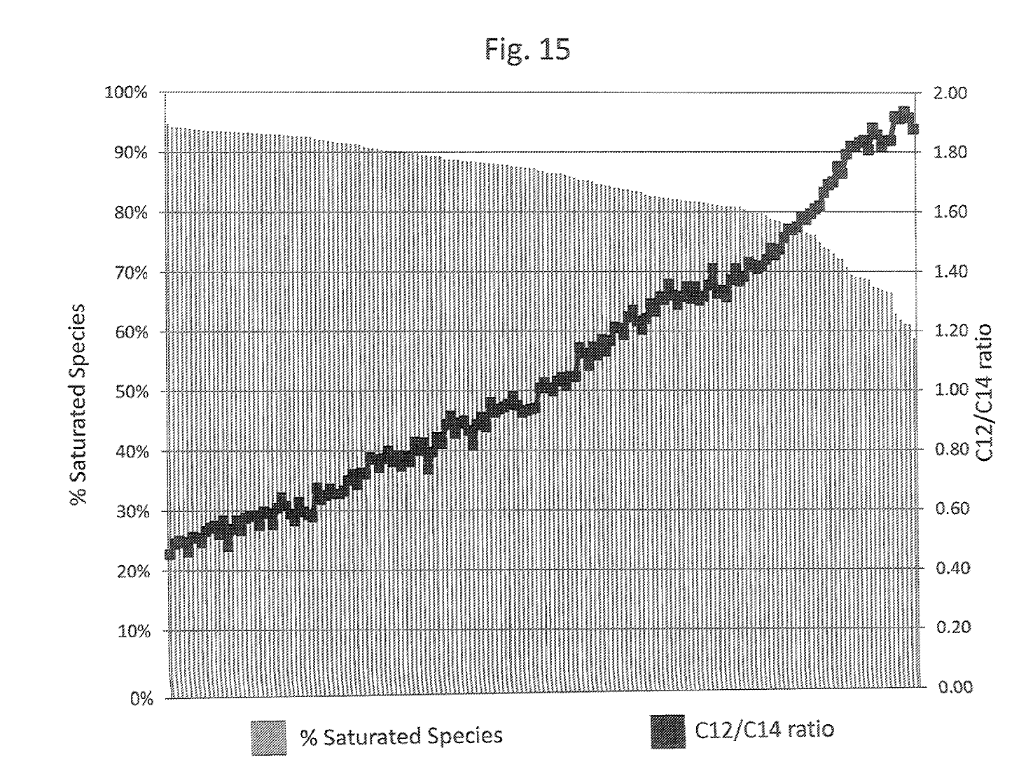

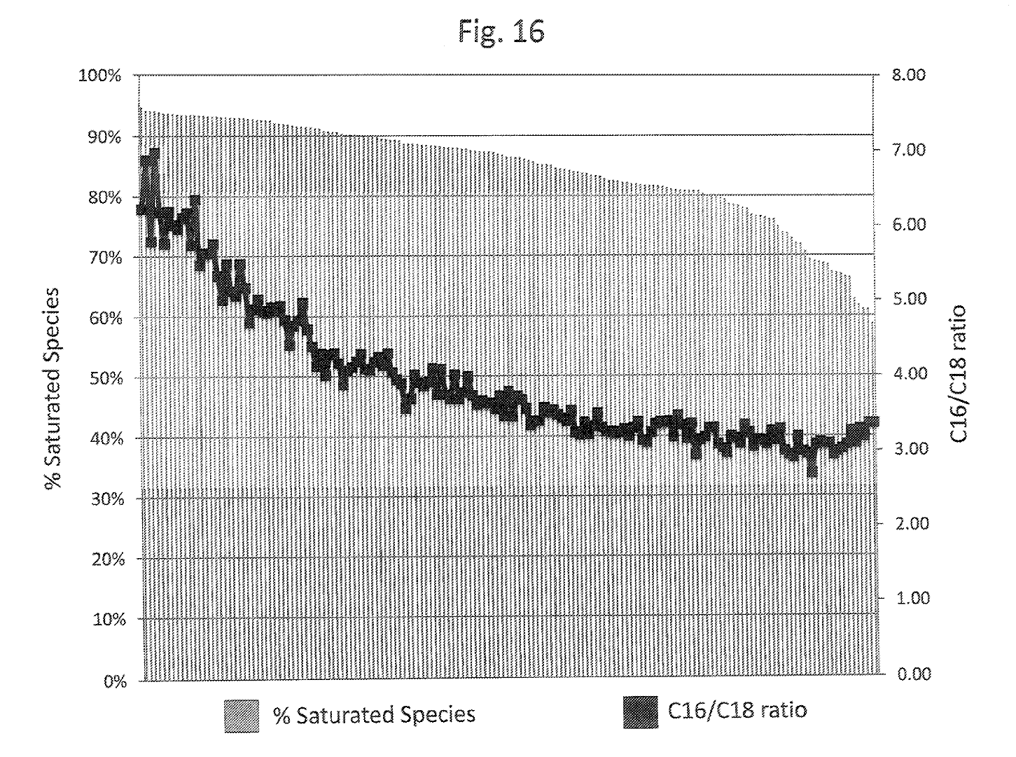

Similar data analyses are shown in FIG. 15 and FIG. 16 for target aliphatic chain lengths characterized by C₁₂/C₁₄ and C₁₆/C₁₈, respectively.

Analyses of the data in the figures demonstrate that the methods of the present invention provide engineered, recombinant microorganisms that produce a wide range of aliphatic chain lengths of fatty acid derivatives, from which one of ordinary skill in the art can select desired target aliphatic chain lengths, with desired levels of saturation. The methods, recombinant microorganisms and cultures of the present invention give one of ordinary skill in the art the tools to tailor aliphatic chain length and saturation to achieve desired results.

### Example 5

### Optimizing Aliphatic Chain Lengths of Fatty Acid Derivatives Using FabA

The data in this example provide a clear illustration of the usefulness of embodiments of the methods of the present invention to make recombinant host cells engineered to produce fatty acid derivatives having targeted aliphatic chain lengths with desired levels of saturation. The example sets forth results of the methods described herein to optimize fatty acid derivative production by optimizing the expression/activities of a β-hydroxyacyl-ACP dehydratase protein (here β-hydroxydecanoyl thioester dehydratase/isomerase protein, the E. coli FabA protein).

Both saturation and chain length of the fatty products can be optimized using the E. coli fabA gene encoding β-hydroxydecanoyl thioester dehydratase/isomerase protein.

An expression plasmid was constructed comprising carB, tesA(12H08), alrAadp1, and fabB(A329G), all expressed under the control of the P_{TRC} promoter. The fabB(A329G) was a glycine for alanine substitution at amino acid position 329 of the E. coli fabB protein. The expression plasmid (designated ALC487) was transformed into strain EG149 (Table 2).

FabA expression was placed under the control of a P_{T5} promoter in strain D178 and the expression plasmid ALC487 was introduced into this strain.

These two strains were screened for the percent saturation of fatty acid derivatives having selected aliphatic chain lengths. The data from this screen demonstrated that modulation of the activity of fabA affected both aliphatic chain length and saturation of fatty acid derivatives.

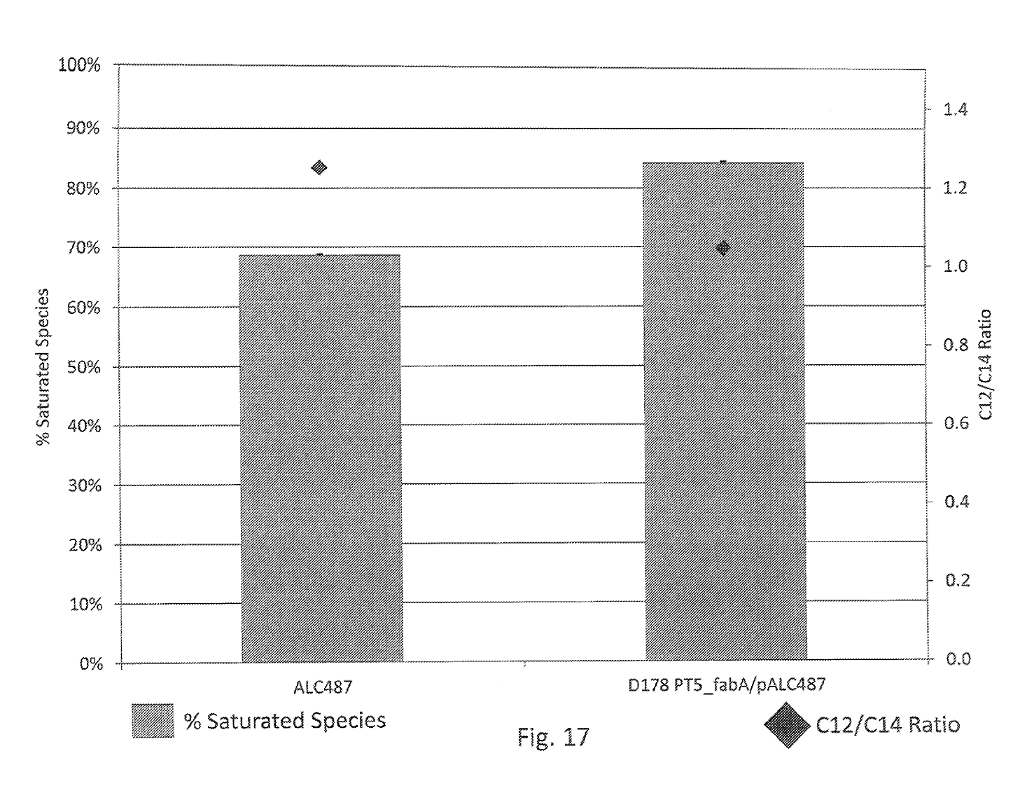

In FIG. 17, data obtained from screening strain EG149 containing the expression plasmid ALC487 is shown as “ALC487.” Data obtained from screening strain D178 containing the expression plasmid ALC487 and having fabA expression under the control of a P_{T5} promoter is shown as “D178 PT5_fabA/pALC487.” As can be seen from the data in the figure, modulation of the expression of fabA resulted in an increase of the saturated species and production of fatty acid derivatives having longer aliphatic chain lengths (based on the C₁₂/C₁₄ ratio).

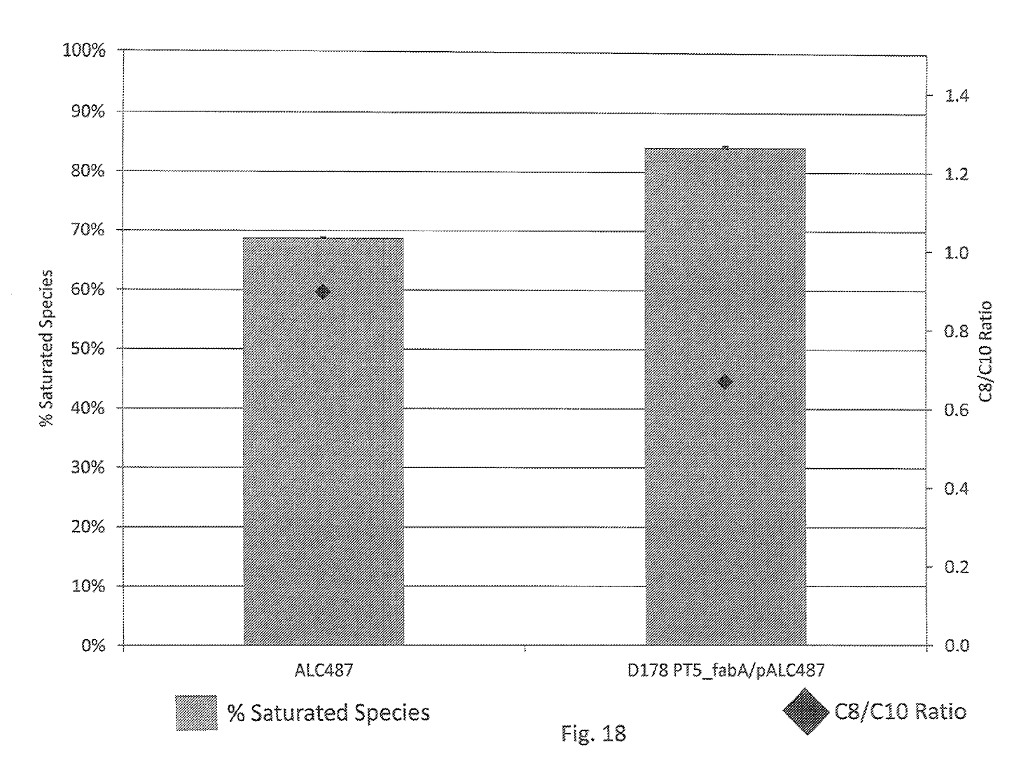

In FIG. 18, data obtained from screening strain EG149 containing the expression plasmid ALC487 is shown as “ALC487.” Data obtained from screening strain D178 containing the expression plasmid ALC487 and having fabA expression under the control of a P_{T5} promoter is shown as “D178 PT5_fabA/pALC487.” As can be seen from the data in the figure, modulation of the expression of fabA resulted in an increase of the saturated species and production of fatty acid derivatives having longer aliphatic chain lengths (based on the C₈/C₁₀ ratio).

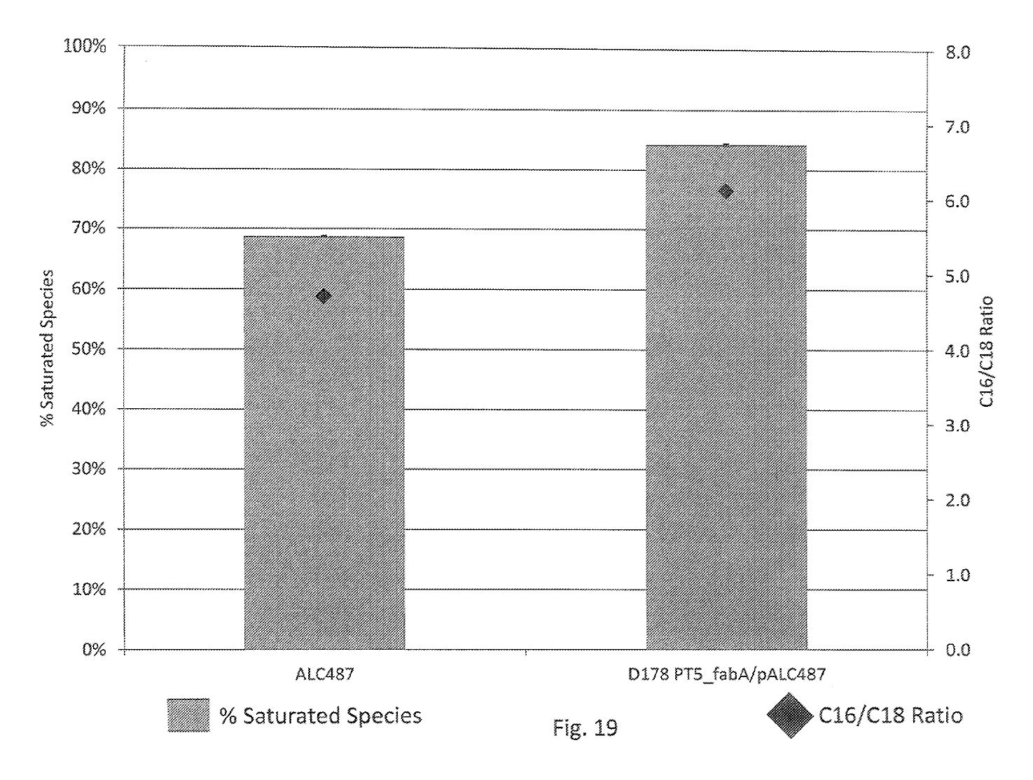

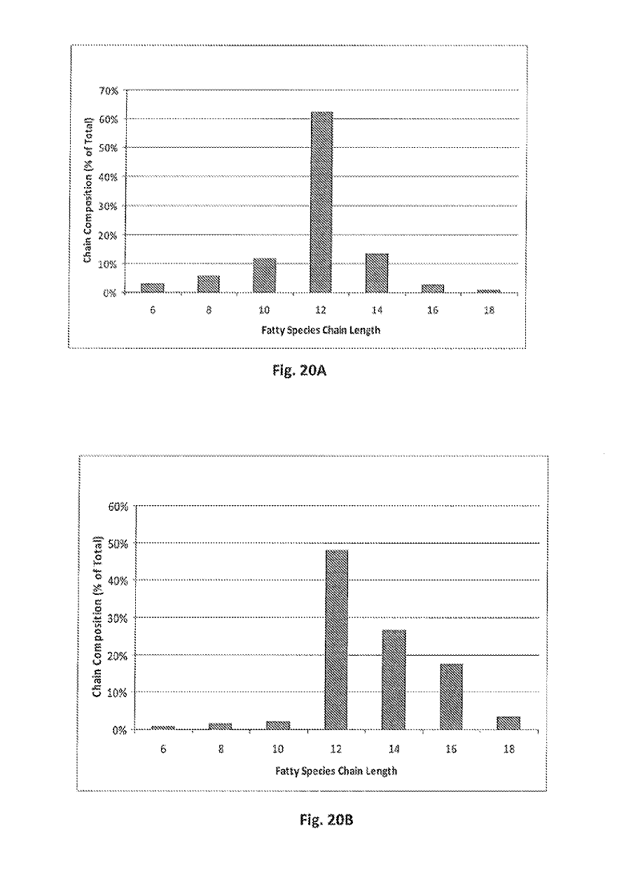

In FIG. 19, data obtained from screening strain EG149 containing the expression plasmid ALC487 is shown as “ALC487.” Data obtained from screening strain D178 containing the expression plasmid ALC487 and having fabA expression under the control of a P_{T5} promoter is shown as “D178 PT5_fabA/pALC487.” As can be seen from the data in the figure, modulation of the expression of fabA resulted an increase of the saturated species and production of fatty acid derivatives having shorter aliphatic chain lengths (based on the C₁₆/C₁₈ ratio).

Analyses of the data in the figures demonstrate that modulation of the activity of fabA provides one of ordinary skill in the art another tool to tailor aliphatic chain length and/or saturation to achieve a desired result.

### Example 6

### Fatty Alcohol Strain Seed Culture Expansion for Developmental Bioreactors

A frozen cell bank vial of the selected E. coli strain was used to inoculate 20 mL of LB broth in a 125 mL baffled shake flask containing spectinomycin antibiotic at a concentration of 115 μg/mL. This shake flask was incubated in an orbital shaker at 32° C. for approximately six hours, then 1.25 mL of the broth was transferred into 125 mL of low P FA2 seed media (2 g/L NH₄Cl, 0.5 g/L NaCl, 3 g/L KH₂PO₄, 1 mM MgSO₄, 0.1 mM CaCl₂, 30 g/L glucose, 1 mL/L of a trace minerals solution (2 g/L of ZnCl₂.4H₂O, 2 g/L of CaCl₂.6H₂O, 2 g/L of Na₂MoO₄.2H₂O, 1.9 g/L of CuSO₄.5H₂O, 0.5 g/L of H₃BO₃, and 10 mL/L of concentrated HCl), 10 mg/L of ferric citrate, 100 mM of Bis-Tris buffer (pH 7.0), and 115 μg/mL of spectinomycin), in a 500 mL baffled Erlenmeyer shake flask, and incubated on a shaker overnight at 32° C.

### A. Bioreactor Fermentation Procedure.

100 mL of this low P FA2 seed culture was used to inoculate a 5 L Biostat Aplus bioreactor (Sartorius BBI), initially containing 1.9 L of sterilized F1 bioreactor fermentation medium. This medium is initially composed of 3.5 g/L of KH₂PO₄, 0.5 g/L of (NH₄)₂SO₄, 0.5 g/L of MgSO₄ heptahydrate, 10 g/L of sterile filtered glucose, 80 mg/L ferric citrate, 5 g/L Casamino acids, 10 mL/L of the sterile filtered trace minerals solution, 1.25 mL/L of a sterile filtered vitamin solution (0.42 g/L of riboflavin, 5.4 g/L of pantothenic acid, 6 g/L of niacin, 1.4 g/L of pyridoxine, 0.06 g/L of biotin, and 0.04 g/L of folic acid), and the spectinomycin at the same concentration as utilized in the seed media. The pH of the culture was maintained at 6.9 using 28% w/v ammonia water, the temperature at 33° C., the aeration rate at 1 lpm (0.5 v/v/m), and the dissolved oxygen tension at 30% of saturation, utilizing the agitation loop cascaded to the DO controller and oxygen supplementation. Foaming was controlled by the automated addition of a silicone emulsion based antifoam (Dow Corning 1410).

A nutrient feed composed of 3.9 g/L MgSO₄ heptahydrate and 600 g/L glucose was started when the glucose in the initial medium was almost depleted (approximately 4-6 hours following inoculation) at an exponential feed rate of 0.3 hr⁻¹ to a constant maximal glucose feed rate of 10-12 g/L/hr, based on the nominal fermentation volume of 2 L. Production of fatty alcohol in the bioreactor was induced when the culture attained an OD of 5 AU (approximately 3-4 hours following inoculation) by the addition of a 1M IPTG stock solution to a final concentration of 1 mM. The bioreactor was sampled twice per day thereafter, and harvested approximately 72 hours following inoculation.

### B. Sample Extraction and Fatty Alcohol/Free Fatty Acid Concentration Analysis.

A 0.5 mL sample of well mixed fermentation broth was transferred into a 15 mL conical tube (VWR), and thoroughly mixed with 5 mL of butyl acetate. The tube was inverted several times to mix, vortexed vigorously for approximately two minutes, then centrifuged for five minutes to separate the organic and aqueous layers. A portion of the organic layer was transferred into a glass vial for gas chromatographic analysis.

### C. Effect of Additional FabB to the Alc-287 Base Strain.

Two strains were tested in bioreactors under identical conditions with (Alc-383) and without (Alc-287) an additional copy of E. coli fabB on the plasmid operon in addition to the native gene copy to ascertain the effect of additional fatty acid biosynthesis capacity on the fermentation results and the resulting product profile. Strain Alc-383 is the Alc-287 base strain with the additional plasmid borne copy of fabB. The primary effects observed based on this increase in the number of copies of fabB were an increase in the amount of product produced and the yield on glucose for Alc-383 in comparison to Alc-287, as well as a change in the product profile toward the production of longer chain alcohols. This lengthening of the chains has the additional effect of reducing the overall saturation of the fatty alcohol product pool.

TABLE 4
FAS Production During Fermentation of Alc-287 and Alc-383

| | | 55 hr | 55 hr | | | |
|---|---|---|---|---|---|---|
| | 55 hr | FAS | FAS | 55 hr | 55 hr | 5.5 hr |
| | FAS | yield on | volumetric | FAS | fatty | FAS |
| Strain | Titer | glucose | productivity | C12/C14 | alcohol | satu- |
| ID | (g/L) | (%) | (g/L/hr) | ratio | (%) | ration |
| Alc-287 | 28.9 | 10.7% | 0.51 | 4.61 | 91.1% | 82.2% |
| Alc-383 | 37.0 | 13.9% | 0.67 | 1.81 | 93.3% | 54.7% |

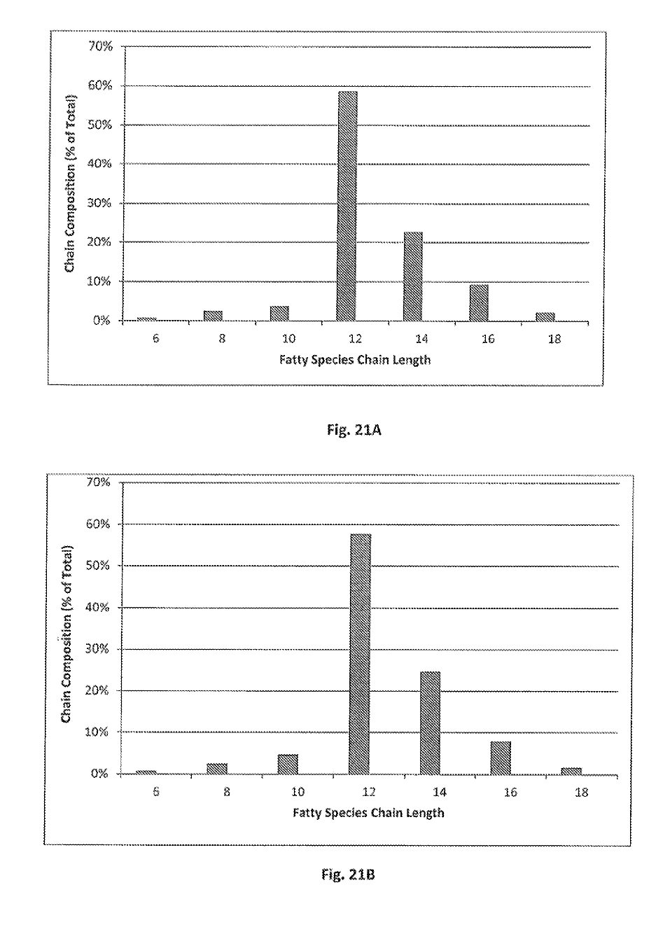

FIGS. 20A-B show the observed differences in chain length distribution that resulted from inclusion of FabB in the Alc-287 base strain.

### D. Effect of Additional TesA to the LC-302 Strain.

Two strains were tested in bioreactors under identical conditions with an additional copy of the 12H08 thioesterase on the chromosome in addition to the one incorporated on the plasmid to ascertain the effect of the additional thioesterase “pull” on the fermentation results and the resulting product profile. Strain LC341 is the LC-302 base strain with the additional chromosomal 12H08 thioesterase. The primary benefit that has been observed with this increase in the thioesterase activity is it increases the amount of product produced and the yield on glucose for a particular strain.

TABLE 5
FAS Production During Fermentation

| | | 58 hr | 58 hr | | | |
|---|---|---|---|---|---|---|
| | 58 hr | FAS | FAS | 58 hr | 58 hr | 58 hr |
| | FAS | yield on | volumetric | FAS | Fatty | FAS |
| Strain | Titer | glucose | productivity | C12/C14 | alcohol | satu- |
| ID | (g/L) | (%) | (g/L/hr) | ratio | (%) | ration |
| LC-302 | 48.6 | 18.7% | 0.84 | 2.6 | 88% | 49% |
| LC-341 | 53.5 | 19.7% | 0.92 | 2.8 | 88% | 53% |

Effect of Adding fabA to the Operon.

The LC-302 parent strain had the fabA gene added to the end of the operon, and three variants of the IGR library were tested (LC-369, LC-372, LC-375) were tested to look at the resulting product profile. The differing intergenic regions of these three strains result in differing amounts of the fabA protein being expressed in the cells. The FAS acronym used below indicates “fatty species”, which is a combination of the fatty alcohol and free fatty acid.

TABLE 6
FAS Production during Fermentation (with FabA added to operon)

| | | 58 hr | 58 hr | | | |
|---|---|---|---|---|---|---|
| | 58 hr | FAS | FAS | 58 hr | 58 hr | 58 hr |
| | FAS | yield on | volumetric | FAS | Fatty | FAS |
| Strain | Titer | glucose | productivity | C12/C14 | alcohol | satu- |
| ID | (g/L) | (%) | (g/L/hr) | ratio | (%) | ration |
| LC-302 | 48.6 | 18.7% | 0.84 | 2.6 | 88% | 49% |
| LC-369 | 47.3 | 17.8% | 0.82 | 2.3 | 89% | 62% |
| LC-372 | 44.3 | 17.2% | 0.76 | 1.7 | 82.7% | 70% |
| LC-375 | 36.7 | 14.4% | 0.63 | 1.5 | 81.5% | 77% |

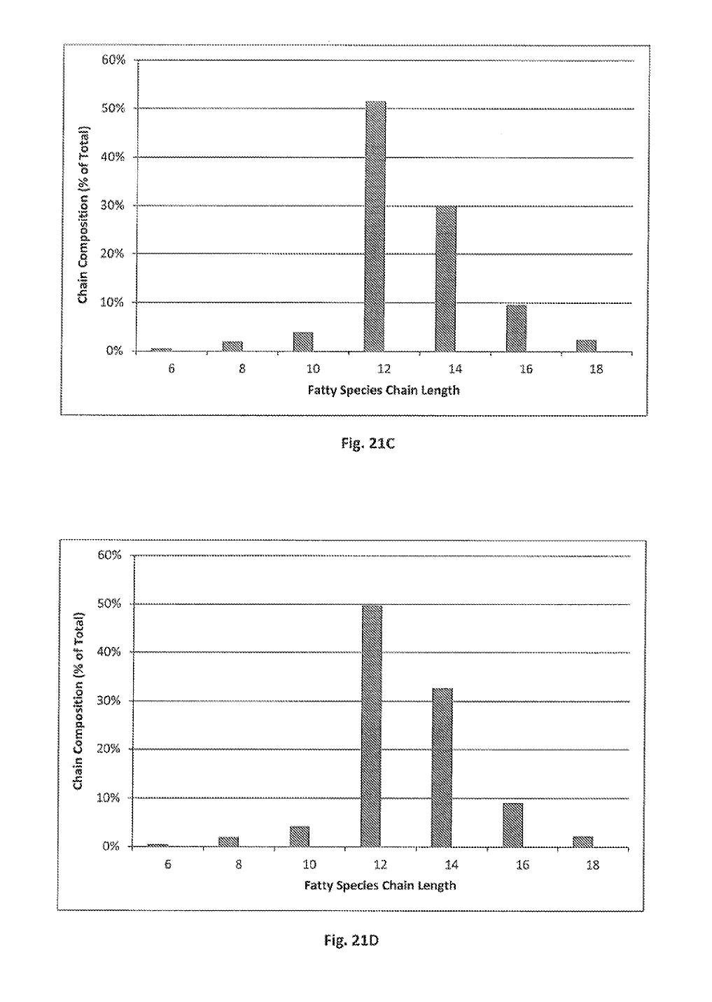

FIGS. 21A-D show the observed differences in chain length distribution that resulted from inclusion of FabA in the operon.

As is apparent to one of skill in the art, various modification and variations of the above aspects and embodiments can be made without departing from the spirit and scope of this invention. Such modifications and variations are within the scope of this invention.

1. A recombinant microorganism comprising a modified activity of a β-hydroxyacyl-ACP dehydratase protein having an Enzyme Commission number of E.C. 4.2.1.- or E.C. 4.2.1.60, wherein said microorganism produces a fatty acid derivative composition having a target aliphatic chain length and/or improved saturation characteristics.

2. The recombinant microorganism of claim 1, wherein
(i) the modified activity differs from an activity of a β-hydroxyacyl-ACP dehydratase protein produced by expression of a starting polynucleotide sequence (SPS_{D}) comprising an open reading frame polynucleotide sequence (ORF_{D}) encoding the β-hydroxyacyl-ACP dehydratase protein, the ORF_{D} having 5′ and 3′ ends, and a 5′ non-coding polynucleotide sequence (NC_{D}) comprising operably-linked regulatory sequences adjacent the 5′-end of the ORF_{D}, in a microorganism of the same kind as the recombinant microorganism; and wherein
(ii) the recombinant microorganism comprises one or more variants of the SPS_{D}, encoding the β-hydroxyacyl-ACP dehydratase protein and operably-linked regulatory sequences, comprising a variant ORF_{D} and/or a variant NC_{D} having less than 100% sequence identity to the ORF_{D} or the NC_{D}, respectively; and wherein
(iii) the fatty acid derivative composition having the target aliphatic chain length produced by the recombinant microorganism comprises a higher titer than a fatty acid derivative composition produced by a the microorganism of the same kind as the recombinant microorganism expressing the SPS_{D}, wherein the ORF_{D} encoding the β-hydroxyacyl-ACP dehydratase protein encodes a protein having an Enzyme Commission number of EC 4.2.1.-.

3. The recombinant microorganism of claim 2, wherein the ORF_{D} encodes an E. coli fabZ derived (3R)-hydroxymyristol acyl carrier protein dehydratase protein that has the sequence set forth in SEQ ID NO: 14, and the variant ORF_{D} encodes a (3R)-hydroxymyristol acyl carrier protein dehydratase protein that has at least about 90% sequence identity to the E. coli fabZ protein (SEQ ID NO:14).

4. The recombinant microorganism of claim 2, wherein the ORF_{D} encoding the β-hydroxyacyl-ACP dehydratase protein encodes a protein having an Enzyme Commission number of EC 4.2.1.60.

5. The recombinant microorganism of claim 4, wherein the ORF_{D} encodes an E. coli fabA derived β-hydroxydecanoyl thioester dehydratase/isomerase protein that has the sequence set forth in SEQ ID NO: 12, and the variant ORF_{D} encodes a β-hydroxydecanoyl thioester dehydratase/isomerase protein that has at least about 90% sequence identity to an E. coli fabA protein (SEQ ID NO: 12).

6. The recombinant microorganism of claim 2, wherein the variant NC_{D} is obtained from a library generated by randomization of the NC_{D}.

7. A recombinant microorganism comprising a modified activity of a β-hydroxyacyl-ACP dehydratase protein that lacks isomerase activity, having an Enzyme Commission number of EC 4.2.1.-, wherein
(i) the modified activity differs from the activity of the β-hydroxyacyl-ACP dehydratase protein that lacks isomerase activity produced by expression of a starting polynucleotide sequence (SSP_{E}) comprising an open reading frame polynucleotide sequence (ORF_{E}) encoding the β-hydroxyacyl-ACP dehydratase protein (FabA/Z) that lacks isomerase activity, the ORF_{E} having 5′ and 3′ ends, and a 5′ non-coding polynucleotide sequence (NC_{E}) comprising operably-linked regulatory sequences adjacent the 5′-end of the ORF_{E}, in a microorganism of the same kind as the recombinant microorganism; and wherein
(ii) the recombinant microorganism comprises one or more polynucleotide sequences, encoding the β-hydroxyacyl-ACP dehydratase protein that lacks isomerase activity and operably-linked regulatory sequences, comprising a variant ORF_{E} and/or a variant NC_{E} having less than 100% sequence identity to the ORF_{E} or the NC_{E}, respectively; -wherein the composition of fatty acid derivatives having the preferred percent saturation produced by the recombinant microorganism comprises a higher titer of fatty acid derivatives having the preferred percent saturation than a fatty acid derivative composition produced by a microorganism of the same kind as the recombinant microorganism expressing the SPS_{E}.

8. The recombinant microorganism of claim 7, wherein the ORF_{E} encodes an E. coli fabZ derived (3R)-hydroxymyristol acyl carrier protein dehydratase protein that has the sequence set forth in SEQ ID NO: 14, and the variant ORF_{E} encodes a (3R)-hydroxymyristol acyl carrier protein dehydratase protein that has at least about 90% sequence identity to an E. coli fabZ protein (SEQ ID NO: 14).

9. The recombinant microorganism of claim 7, wherein the variant NC_{E} is obtained from a library generated by randomization of the NC_{E}.

10. The recombinant microorganism of claim 7, further comprising one or more polynucleotide sequences having an open reading frame encoding an elongation β-ketoacyl-ACP synthase protein, the protein having an Enzyme Commission number of EC 2.3.1.-, and operably-linked regulatory sequences.

11. The recombinant microorganism of claim 7, further comprising one or more polynucleotide sequences having an open reading frame encoding a thioesterase, the protein having an Enzyme Commission number of EC 3.1.1.5 or EC 3.1.2.-, and operably-linked regulatory sequences.

12. The recombinant microorganism of claim 7, further comprising one or more polynucleotide sequences having an open reading frame encoding a carboxylic acid reductase protein, having an Enzyme Commission number of EC 6.2.1.3 or EC 1.2.1.42, and operably-linked regulatory sequences.

13. The recombinant microorganism of claim 1, further comprising one or more polynucleotide sequences having an open reading frame encoding a thioesterase, the protein having an Enzyme Commission number of EC 3.1.1.5 or EC 3.1.2.-, and operably-linked regulatory sequences.

14. The recombinant microorganism of claim 7, wherein the recombinant microorganism is a bacterium.

15. The recombinant microorganism culture of claim 14, wherein the bacterium is Escherichia coli.

16.-88. (canceled)

89. The recombinant microorganism of claim 1, wherein the recombinant microorganism is a bacterium.

90. The recombinant microorganism culture of claim 89, wherein the bacterium is Escherichia coli.
